(19) United States
(12) Patent Application Publication
(10) Pub. No.: US 2024/0316166 A1
(43) Pub. Date: Sep. 26, 2024

(54) VACCINE COMPOSITIONS AND METHODS OF USING THE SAME

(71) Applicant: The Board of Supervisors of Louisiana State University and Agricultural and Mechanical College, Baton Rouge, LA (US)

(72) Inventor: Hong Xin, New Orleans, LA (US)

(21) Appl. No.: 18/368,505

(22) Filed: Sep. 14, 2023

Related U.S. Application Data
(63) Continuation-in-part of application No. 18/143,046, filed on May 3, 2023, now abandoned.
Application No. 18/143,046 is a division of application No. 16/722,846, filed on Dec. 20, 2019, now Pat. No. 11,707,511.
Application No. 16/722,846 is a continuation-in-part of application No. PCT/US2018/038512, filed on Jun. 20, 2018.
(60) Provisional application No. 62/522,217, filed on Jun. 20, 2017.

(51) Int. Cl. A61K 39/00; A61K 39/395; C07K 14/40

(52) U.S. Cl. CPC A61K 39/0002; A61K 39/39575; C07K 14/40; A61K 2039/505; A61K 2039/55505; A61K 2039/575; C07K 2317/24; C07K 2319/40

(57) ABSTRACT

This invention is directed to vaccine compositions and methods of using the same to prevent infection.

This application is a Continuation-In-Part of patent application Ser. No. 18/143,046, filed on May 3, 2023, which is a Divisional of patent application of Ser. No. 16/722,846, filed on Dec. 20, 2019, which is a Continuation-In-Part of International Patent Application No. PCT/US2018/038512, filed on Jun. 20, 2018, which claims priority from U.S. Provisional Patent Application No. 62/522,217, filed on Jun. 20, 2017, the contents of each of which are incorporated herein by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under Grant No. R03 AI107536 and R21 AI168929 awarded by the National Institutes of Health. The government has certain rights in the invention.

### REFERENCE TO A SEQUENCE LISTING XML

This application contains a Sequence Listing which has been submitted electronically in XML format. The Sequence Listing XML is incorporated herein by reference. Said XML file, created on Feb. 9, 2024, is named 2932719-000012-US4_SL.xml and is 267,131 bytes in size.

### FIELD OF THE INVENTION

This invention is directed to vaccine compositions and methods of using the same to prevent infection.

### BACKGROUND OF THE INVENTION

Morbidity and mortality from invasive fungal infections remain unacceptably high despite availability of antifungal agents, underscoring the need for more effective preventative strategies. Invasive fungal diseases often take hold when a person's natural defenses are weakened. These infections frequently occur in hospital settings. Vaccination of high-risk groups is a promising strategy to prevent invasive fungal infections because identifiable risk factors are defined for many such infections. Development of these risk factors can precede infection, affording a window of opportunity to vaccinate acutely at-risk patients before the onset of infection.

### SUMMARY OF THE INVENTION

The present invention provides an isolated monoclonal antibody or antigen-binding fragment thereof. In embodiments, the isolated monoclonal antibody or antigen-binding fragment thereof comprises a heavy chain, light chain, or both a heavy chain and a light chain, wherein the heavy chain comprises a CDR1 comprising SYWMS (SEQ ID NO: 134), NYWMN (SEQ ID NO: 137), SYTMH (SEQ ID NO: 140), SYGVH (SEQ ID NO: 143), DYSMH (SEQ ID NO: 146), EYTMH (SEQ ID NO: 149), GYYMH (SEQ ID NO: 152), or DFSMH (SEQ ID NO: 155), CDR2 comprising RIDPYDSETHYNQKFKD (SEQ ID NO: 135), EIRLKSNNYATHYAESVKG (SEQ ID NO: 138), YINPSSGYTDYNQKFKD (SEQ ID NO: 141), VIWSGGTTDYNAAFIS (SEQ ID NO: 144), WINTETGEPTYADDFKG (SEQ ID NO: 147), GINPNNGGTRYNQKFKG (SEQ ID NO: 150), RINPYTGATSYTQNFKD (SEQ ID NO: 153), or WINTETVEPTYADDFKG (SEQ ID NO: 156), CDR3 comprising TAASFDY (SEQ ID NO: 136), GNY, LYDNYDYYAMDY (SEQ ID NO: 142), GGHRGFAY (SEQ ID NO: 145), NYYDTTGFAY (SEQ ID NO: 148), YFPY (SEQ ID NO: 151), GGGRSSYWYFDV (SEQ ID NO: 154), or VSYDGYYGDFAMDY (SEQ ID NO: 157), or a combination of CDRs thereof, and wherein the light chain comprises a CDR1 comprising RSSQSLVHSNGNSYLH (SEQ ID NO: 158), SASSTISSNYLH (SEQ ID NO: 161), KSSQSLLNSRIRKNYLA (SEQ ID NO: 164), KASQDVGTAVA (SEQ ID NO: 167), KSSQRLLYSYGKTYLN (SEQ ID NO: 170), RASQSIGTSIH (SEQ ID NO: 173), RSSQSLLDSDGKTYLN (SEQ ID NO: 176), SASQAISNYLN (SEQ ID NO: 179), or RASQDISNYLN (SEQ ID NO: 182), CDR2 comprising KVSNRFS (SEQ ID NO: 159), RTSNLAS (SEQ ID NO: 162), WASTRES (SEQ ID NO: 165), WASTRHT (SEQ ID NO: 168), LVSKLDS (SEQ ID NO: 171), FASESIS (SEQ ID NO: 174), LVSKLDS (SEQ ID NO: 177), YTSSLHS (SEQ ID NO: 180), or YTSRLHS (SEQ ID NO: 183), CDR3 comprising SQSTHVPFT (SEQ ID NO: 160), QQGSTISRT (SEQ ID NO: 163), KQSYNLLT (SEQ ID NO: 166), QQYSSYPLT (SEQ ID NO: 169), VQDTHFPYT (SEQ ID NO: 172), QQSNSWPTYT (SEQ ID NO: 175), WQGTHFPWT (SEQ ID NO: 178), QQYSKLPWT (SEQ ID NO: 181), or QQGNTLPWT (SEQ ID NO: 184), or a combination of CDRs thereof.

In embodiments, the antibody is fully human or humanized.

In embodiments, the antibody is an IgG.

In embodiments, the antibody is a single chain antibody.

In embodiments, the antibody further comprises a heavy chain constant region, a light chain constant region, an Fc region, or a combination thereof.

In embodiments, the antibody comprises 2B10C1, 1D4H5, 2D5F7, 10E7E2, 7C6E8, 9F2G5, 6H1G8, or 7H6A2.

In embodiments, the antibody competes with binding of 2B10C1, 1D4H5, 2D5F7, 10E7E2, 7C6E8, 9F2G5, 6H1G8, or 7H6A2.

In embodiments, the antibody or fragment is linked to a therapeutic agent.

In embodiments, the antibody is a single chain fragment.

Aspects of the invention are further drawn towards an isolated antibody or fragment thereof comprising a VH CDR1 comprising the amino acid sequence of SYWMS (SEQ ID NO: 134), a VH CDR2 comprising the amino acid sequence of RIDPYDSETHYNQKFKD (SEQ ID NO: 135), a VH CDR3 comprising the amino acid sequence of TAASFDY (SEQ ID NO: 136), a VL CDR1 comprising the amino acid sequence of RSSQSLVHSNGNSYLH (SEQ ID NO: 158), a VL CDR2 comprising the amino acid sequence of KVSNRFS (SEQ ID NO: 159), and a VL CDR3 comprising the amino acid sequence of SQSTHVPFT (SEQ ID NO: 160); or a VH CDR1 comprising the amino acid sequence of NYWMN (SEQ ID NO: 137), a VH CDR2 comprising the amino acid sequence of EIRLKSNNYATHYAESVKG (SEQ ID NO: 138), a VH CDR3 comprising the amino acid sequence of GNY, a VL CDR1 comprising the amino acid sequence of SASSTISSNYLH (SEQ ID NO: 161), a VL CDR2 comprising the amino acid sequence of RTSNLAS (SEQ ID NO: 162), and a VL CDR3 comprising the amino acid sequence of QQGSTISRT (SEQ ID NO: 163); or a VH CDR1 comprising the amino acid sequence of SYTMH (SEQ ID NO: 140), a VH CDR2 comprising the amino acid sequence of YINPSSGYTDYNQKFKD (SEQ ID NO: 141), a VH CDR3 comprising the amino acid sequence of LYDNYDYYAMDY (SEQ ID NO: 142), a VL CDR1 comprising the amino acid sequence of KSSQSLLNSRIRKNYLA (SEQ ID NO: 164), a VL CDR2 comprising the amino acid sequence of WASTRES (SEQ ID NO: 165), and a VL CDR3 comprising the amino acid sequence of KQSYNLLT (SEQ ID NO: 166); or a VH CDR1 comprising the amino acid sequence of SYGVH (SEQ ID NO: 143), a VH CDR2 comprising the amino acid sequence of VIWSGGTTDYNAAFIS (SEQ ID NO: 144), a VH CDR3 comprising the amino acid sequence of GGHRGFAY (SEQ ID NO: 145), a VL CDR1 comprising the amino acid sequence of KASQDVGTAVA (SEQ ID NO: 167), a VL CDR2 comprising the amino acid sequence of WASTRHT (SEQ ID NO: 168), and a VL CDR3 comprising the amino acid sequence of QQYSSYPLT (SEQ ID NO: 169); or a VH CDR1 comprising the amino acid sequence of DYSMH (SEQ ID NO: 146), a VH CDR2 comprising the amino acid sequence of WINTETGEPTYADDFKG (SEQ ID NO: 147), a VH CDR3 comprising the amino acid sequence of NYYDTTGFAY (SEQ ID NO: 148), a VL CDR1 comprising the amino acid sequence of KSSQRLLYSYGKTYLN (SEQ ID NO: 170), a VL CDR2 comprising the amino acid sequence of LVSKLDS (SEQ ID NO: 171), and a VL CDR3 comprising the amino acid sequence of VQDTHFPYT (SEQ ID NO: 172), or a VL CDR1 comprising the amino acid sequence of RASQSIGTSIH (SEQ ID NO: 173), a VL CDR2 comprising the amino acid sequence of FASESIS (SEQ ID NO: 174), and a VL CDR3 comprising the amino acid sequence of QQSNSWPTYT (SEQ ID NO: 175); or a VH CDR1 comprising the amino acid sequence of EYTMH (SEQ ID NO: 149), a VH CDR2 comprising the amino acid sequence of GINPNNGGTRYNQKFKG (SEQ ID NO: 150), a VH CDR3 comprising the amino acid sequence of YFPY (SEQ ID NO: 151), a VL CDR1 comprising the amino acid sequence of RSSQSLLDSDGKTYLN (SEQ ID NO: 176), a VL CDR2 comprising the amino acid sequence of LVSKLDS (SEQ ID NO: 177), and a VL CDR3 comprising the amino acid sequence of WQGTHFPWT (SEQ ID NO: 178); or, a VH CDR1 comprising the amino acid sequence of GYYMH (SEQ ID NO: 152), a VH CDR2 comprising the amino acid sequence of RINPYTGATSYTQNFKD (SEQ ID NO: 153), a VH CDR3 comprising the amino acid sequence of GGGRSSYWYFDV (SEQ ID NO: 154), a VL CDR1 comprising the amino acid sequence of SASQAISNYLN (SEQ ID NO: 179), a VL CDR2 comprising the amino acid sequence of YTSSLHS (SEQ ID NO: 180), and a VL CDR3 comprising the amino acid sequence of QQYSKLPWT (SEQ ID NO: 181); or a VH CDR1 comprising the amino acid sequence of DFSMH (SEQ ID NO: 155), a VH CDR2 comprising the amino acid sequence of WINTETVEPTYADDFKG (SEQ ID NO: 156), a VH CDR3 comprising the amino acid sequence of VSYDGYYGDFAMDY (SEQ ID NO: 157), a VL CDR1 comprising the amino acid sequence of RASQDISNYLN (SEQ ID NO: 182), a VL CDR2 comprising the amino acid sequence of YTSRLHS (SEQ ID NO: 183), and a VL CDR3 comprising the amino acid sequence of QQGNTLPWT (SEQ ID NO: 184).

Aspects of the invention are further drawn towards an isolated antibody or fragment thereof comprising a heavy chain variable region comprising an amino acid sequence selected from the group consisting of SEQ ID NOS: 60, 64, 68, 72, 108, 111, 113, or 115, or a sequence at least 90% identical thereto.

Further, aspects of the invention are drawn towards an isolated antibody or fragment thereof comprising a light chain variable region comprising an amino acid sequence selected from the group consisting of SEQ ID NOS: 62, 66, 70, 74, 109, 110, 112, 114, or 116, or a sequence at least 90% identical thereto.

Still further, aspects of the invention are drawn towards an isolated antibody or fragment thereof comprising a heavy chain variable region comprising an amino acid sequence selected from the group consisting of SEQ ID NOS: 60, 64, 68, 72, 108, 111, 113, or 115, or a sequence at least 90% identical thereto, and a light chain variable region comprising an amino acid sequence selected from the group consisting of SEQ ID NOS: 62, 66, 70, 74, 109, 110, 112, 114, or 116, or a sequence at least 90% identical thereto.

Aspects of the invention are also drawn towards an isolated monoclonal antibody or antigen-binding fragment thereof comprising a heavy chain, a light chain, or a combination thereof. In embodiments, the heavy chain comprises an amino acid sequence about 95% identical to SEQ ID NO: 60, and the light chain comprises an amino acid sequence about 95% identical to SEQ ID NO: 62; or the heavy chain comprises an amino acid sequence about 95% identical to SEQ ID NO: 64, and the light chain comprises an amino acid sequence about 95% identical to SEQ ID NO: 66; or the heavy chain comprises an amino acid sequence about 95% identical to SEQ ID NO: 68, and the light chain comprises an amino acid sequence about 95% identical to SEQ ID NO: 70; or the heavy chain comprises an amino acid sequence about 95% identical to SEQ ID NO: 72, and the light chain comprises an amino acid sequence about 95% identical to SEQ ID NO: 74; or the heavy chain comprises an amino acid sequence about 95% identical to SEQ ID NO: 108, and the light chain comprises an amino acid sequence about 95% identical to SEQ ID NO: 109 or SEQ ID NO: 110; or the heavy chain comprises an amino acid sequence about 95% identical to SEQ ID NO: 111, and the light chain comprises an amino acid sequence about 95% identical to SEQ ID NO: 112; or the heavy chain comprises an amino acid sequence about 95% identical to SEQ ID NO: 113, and the light chain comprises an amino acid sequence about 95% identical to SEQ ID NO: 114; or the heavy chain comprises an amino acid sequence about 95% identical to SEQ ID NO: 114, and the light chain comprises an amino acid sequence about 95% identical to SEQ ID NO: 116.

Aspects of the invention are also drawn towards a nucleic acid encoding the antibody or fragment as described herein, a vector comprising the nucleic acid, and/or a cell comprising the vector.

Also, aspects of the invention are drawn towards a pharmaceutical composition comprising the antibody or fragment thereof as described herein, and a pharmaceutically acceptable carrier or excipient.

Aspects of the invention are drawn towards an isolated cell comprising one or more polynucleotide(s) encoding the antibody or fragment thereof.

Aspects of the invention are drawn towards a method of treating or preventing a microbial infection. In embodiments, the method comprises administering to the subject the antibody or fragment as described herein.

In embodiments, the microbial infection comprises fungi. For example, the fungi comprises Candida spp.

Other objects and advantages of this invention will become readily apparent from the ensuing description.

### BRIEF DESCRIPTION OF THE FIGURES

FIG. 1 shows the examples of target populations for anti-fungal vaccines and monoclonal antibodies.

FIG. 2 shows peptide candidates Met6-2, FbaU1 induced high degree protection against disseminated candidiasis caused by C. albicans in mice. Peptides Met6-2, Fba3 and FbaU1 induced good protective responses against disseminated candidiasis caused by C. albicans (panels A and C, respectively), and the protected groups had reduced or non-detectable CFUs in their kidneys as compared to controls (panels B and D, respectively).

FIG. 3 shows the range of protection by universal peptide vaccines can be broadened to protect against disseminated candidiasis caused by C. tropicalis. Peptide FbaU1 induced the best protection (100% survival) among the peptide candidates; both Fba3 and Met6-2 peptides induced good protection with 80% survival up to 80 days (Panel A). CFU data in kidneys of immunized groups are very consistent with the survival data (Panel B), FbaU1, Fba3 and Met6-2 groups had significantly reduced live fungal cells in their kidneys as compared to controls (p<0.001).

FIG. 4 shows the range of protection by universal peptides can be broadened to protect against disseminated candidiasis caused by C. glabrata. Peptide FbaU1& Met6-2 peptides were able to induce a high degree protection.

FIG. 5 shows that, when tested in C57BL/6 mice, the tested peptide vaccines are prone to Th1 responses.

FIG. 6 shows passive immunization with mAbs E2-9 and M2-4 enhanced resistance of neutropenic mice to disseminated candidiasis. (A) Neutropenic mice received 0.5 ml of either mAb M2-4 or mAb E2-9 (one dose) had significantly prolonged survival as compared to control animals that received DPBS or absorbed mAbs, p<0.05; (B) Consistently, the groups received treatment of either protective mAb (M2-4 and E2-9) had significantly reduced CFUs in kidneys compared with control mice, p<0.001. (C) After challenge, some groups of mice were given the same dose of antibodies (mAb E2-9 or MAb M2-4) as above or control materials on every other day post-challenge for two weeks. The survival of recipients was significantly improved (p<0.01). As compared to one-dose treatment, treatment with mAb M2-4 for two weeks was able to increase protection from 20% to 40%; with MAB E2-9, from 20% to 60%. (D) Groups received mAb treatment for two weeks had significantly reduced CFUs in kidneys as compared with control animals that received DPBS or absorbed mAbs (p<0.001).

FIG. 7 shows passive transfer of immune sera.

FIG. 8 shows main prevention strategies.

FIG. 9 shows a schematic of an embodiment comprising a synthetic chimeric peptide vaccine against invasive fungal infections.

FIG. 10 shows a neutropenic mouse model of human disseminated candidiasis.

FIG. 11 shows the effect of immunosuppression on kidney fungal burden (NAC).

FIG. 12 shows a neutropenic mouse model of NAC.

FIG. 13 shows double peptide conjugate vaccines induced protection against disseminated candidiasis caused by medically important Candida species.

FIG. 14 shows protective mAbs protect immunocompromised mice against disseminated infection by Candida auris. Mice received either mAb 7H6A2 or 1D4H5 or the two mAbs in combination had reduced CFUs in kidneys (A) and in brains (B) as compared with control mice.

FIG. 15 shows monoclonal antibodies (mAbs) are effective against invasive candidiasis caused by C. auris, the multidrug-resistant Candida species. A. Complement protein C5-deficient A/J mice were treated with one of the five mAb or with DPBS as a vehicle control. The following day, mice were challenged with 1×10⁸ C. auris cells/100 ul DPBS dose per mouse. Mice treated with mAbs 6H1G8 or 9F2G5 showed 50% survival by the termination of the experiment (40 days post challenge), whereas mice treated with 3H7E3 showed 50% survival up until 37 days post challenge. Groups treated with 10E7E2 or 3H7E3 showed 25% survival. B. Fungal burdens in the kidneys (top), heard (center), and brain (bottom). Non-detectible levels of fungal burden were achieved in the kidneys of one 6H1G8-treated mouse, whereas non-detectible levels of fungal burden were achieved in the heart and brain of several mice treated with 6H1G8, 9F2G5, or 3H7E3.

FIG. 16 shows two protective mAbs in combination conferred improved protection against systemic C. auris infection in passive transfer experiments. Identified protective mAbs were obtained by use of standard hybridoma technique and purified by use of protein A/G. A/J mice were given an i.p. dose of either single mAb or two mAbs in combination 24 hours before hematogensous challenge with a lethal dose (2×10⁸) of C. auris AR-0386 live cells. Mice received either 9G2F5 alone or 6H1G8 alone or 9F2G5 and 3H7E3 in combination had 40% survival rate respectively, with prolonged survival as compared to control animals that received DPBS buffer control.

FIG. 17 shows experimental protocol.

FIG. 18 shows mAb 9F2G5 used alone or with antifungal drug has therapeutic activity against C. auris disseminated candidiasis. Administration of 9F2G5 (α-Pgk1 peptide, IgG2a) alone provide better protection as compared to Micafungin alone therapeutic treatment, evidenced by reduced fungal burdens in both kidney (A) and heart (B) at day 4 post-infection. Antibody 9F2G5 given by i.p. 24 hours before challenge, combined with Micafungin given i.p. 24h post-infection had enhanced therapeutic potential of Micafungin, reducing CFUs in both kidney (A) and heart (B) significantly as compared to Micafungin alone therapeutic treatment.

FIG. 19 shows the homology of cell surface epitopes targeted by protective mAbs among medically significant Candida species. *Peptides found in C. albicans cell wall proteins and conjugated each peptide, originally intended to act as a carrier to the fungal cell wall β-1,2-trisaccharide [β-(Man)₃] to create glycopeptide conjugate vaccines. Four peptides (Fba, Met6, PGK1 and Hwp1) served well as effective carriers for the protective glycan epitope, turned out to be excellent vaccines by themselves in that they also induced protection against invasive candidiasis in mice. We further demonstrated that antibodies specific for the peptides are each protective. Uniquely, the protective mAbs targeting each of four peptide epitopes were also expressed 85%-100% by other medically important Candida species in addition to C. albicans (see FIG. 19). Without wishing to be bound by theory, these “universal” mAbs can protect broadly against other Candida species.

FIG. 20 shows protective efficacy of Pgk1-specific monoclonal antibodies against disseminated C. auris infection. 7 week male A/J mice; Candida auris challenge strain: CDC AR-0386, 1×10⁸ cells. Passive transfer of 9F2 mAb provided great protection evidenced by survival rate, survival time and reduced fungal burdens in targeted organ. We conclude passive transfer of universal Candida peptide-specific mAb 9F2 (anti-Pgk1) provides significantly extended survival and reduced fungal burdens in the heart of C. auris-infected mice compared to control mice.

FIG. 21 shows in vivo protective efficacy of monoclonal antibody cocktails. For example, we evaluated prophylactic efficacy of mAb cocktail 6H1+9F2 against C. auris disseminated infection in A/J mice. Prophylactic Treatment Study (n=5)—Passive mAb Transfer: 6H1 (anti-Hwp1, IgG2b): 2× (400 μL); 9F2 (anti-Pgk1, IgG2a): 1× (200 μL); 6H1+9F2 (2×400 μL 6H1+1×200 μL 9F2); Negative Control: DPBS; Challenge: Sub-lethal Dose: 4×10⁷ AR-0386; 35-Day Survival & Fungal Burdens. Combination extends survival better than individual treatment and reduces burdens better than individual treatment (brain and heart); Kidney—Combo vs 9F2, p=0.0031; Brain-Combo vs DPBS, p=0.0488, Combo vs 9F2, p=0.0252; Heart (none are significant). Accordingly, individually protective mAbs, for example, 6H1 and 9F2, provide greater protection against disseminated C. auris infection when administered as a cocktail, as evidenced by extended survival and reduced fungal burdens.

FIG. 22 shows antibody-mediated growth inhibition. We evaluated if 9F2 mAb binding inhibits proliferation of C. auris yeast cells using an in vitro assay. C. auris yeast was incubated with mAb for 18 hours, plated onto GYEP agar plates, and CFUs were quantified. ND means non-detectable.

FIG. 23 shows antibody-mediated growth inhibition—dose response.

FIG. 24 shows mAb 9F2-mediated growth inhibition for C. auris isolates AR-381, AR-382, AR-383, AR-384, and AR-385.

FIG. 25 shows mAbs 9F2, G11.2 and C3.1-mediated growth inhibition/killing for C. albicans. SC5314 is the most commonly used clinical isolate.

FIG. 26 shows mAbs 9F2, G11.2, and C3.1-mediated growth inhibition and fungicidal activity for C. glabrata. Candida glabrata isolate is ATCC 15126.

FIG. 27 shows mAbs 9F2, G11.2, 7C6E8 and C3.1-Mediated Growth Inhibition and fungicidal activity for Candida parapsilosis. Candida parapsilosis isolate is ATCC 22519.

FIG. 28 shows mAbs 9F2, G11.2, 7C6E8 and C3.1-Mediated Growth Inhibition and fungicidal activity for Candida tropicalis. Candida tropicalis isolate is ATCC 66029.

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the term “universal,” for example “universal peptide” or “antigenic universal peptide” or “universal peptide vaccine,” can refer to the ability to protect against, prevent, delay the onset of or treat infection, or symptoms thereof, caused by different microorganisms. For example, a universal peptide as described herein can protect against infections caused by various species of fungi, bacteria, viruses, and/or protozoa. Non-limiting examples of species of fungi which can cause infections comprise Candida, Aspergillus, Cryptococcus, Trichophyton, Microsporum, Epidermophyton, Rhizopus, Mucor, Cunninghamella, Apophysomyces, Lichtheimia, Exserohilum, Cladosporium, Blastomyces, Coccidioides, Histoplasma, Pneumocystis, and/or Sporothrix.

Non-limiting examples of species of bacteria which can cause infections comprise, such as Eschericia, Salmonella, Helicobacter, Neisseria, Staphylococcus, Streptococcus, Campylobecter, Clostridium, Listeria, Vibrio Chlamydia, and/or Treponema.

Non-limiting examples of viruses which can cause infections comprise varicella zoster virus, human immunodeficiency virus, influenza virus, herpes virus, human papillomavirus, Epstein-Barr virus, mumps virus, rubeola virus, rotavirus, norovirus, West nile virus, Ebola virus, Respiratory syncytial virus, coronavirus, rhinovirus, parainfluenza virus, and/or adenovirus.

Non-limiting examples of species of protozoa which can cause infections comprise plasmodium, trypanosome, amoeba, giardia, a species of Acanthamoeba. Non-limiting examples of disease caused by protozoal infections comprise Acanthamoeba keratitis, malaria, protozoal pneumonia.

### Microbial Infections

The term “microbial infection” can refer to the invasion of the host mammal by pathogenic microbes or microorganisms capable of causing an infection. This includes the excessive growth of microbes which are normally present in or on the body of a mammal. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the effects of the presence of a microbial population(s) is damaging the cells or other tissue of a mammal.

Non-limiting examples of microorganisms capable of causing an infection comprise fungi, bacteria, virus, and protozoa.

For example, microbial infections can be caused by species of fungi such as Candida, Aspergillus, Cryptococcus, Trichophyton, Microsporum, Epidermophyton, Rhizopus, Mucor, Cunninghamella, Apophysomyces, Lichtheimia, Exserohilum, Cladosporium, Blastomyces, Coccidioides, Histoplasma, Pneumocystis, and/or Sporothrix.

In some embodiments, microbial infections can be caused by species of bacteria, such as Eschericia, Salmonella, Helicobacter, Neisseria, Staphylococcus, Streptococcus, Campylobecter, Clostridium, Listeria, Vibrio Chlamydia, and/or Treponema.

For example, microbial infections can be caused by viruses, such as varicella zoster virus, human immunodeficiency virus, influenza virus, herpes virus, human papillomavirus, Epstein-Barr virus, mumps virus, rubeola virus, rotavirus, norovirus, West nile virus, Ebola virus, Respiratory syncytial virus, coronavirus, rhinovirus, parainfluenza virus, and/or adenovirus.

In some embodiments, the protozoan that can cause microbial infection comprises Plasmodium, trypanosome, amoeba, giardia, a species of Acanthamoeba. Non-limiting examples of disease caused by protozoal infections comprise Acanthamoeba keratitis, malaria, protozoal pneumonia.

### Invasive Fungal Infections

The polymorphic fungus Candida albicans is a commensal organism that colonizes the gastrointestinal tract, vagina and some cutaneous areas of the majority of healthy humans. However, under certain conditions the fungus is able to cause a variety of infections, ranging from mucosal to life-threatening invasive candidiasis. C. albicans continues to be the most common cause of various forms of candidiasis, but several other Candida spp. are also important agents. Invasive disease is associated with billions of dollars each year in healthcare costs and a mortality rate estimated at ˜40%. The limited number and toxicity of antifungal agents, and, most importantly, the poor outcome of almost half of the number of candidemia patients treated with appropriate antifungal therapy, militates in favor of disease prevention, possibly through active and passive immunization strategies.

Fungal infections, such as Candida, commonly afflict patients in the ICU, and also those subjects with suppressed immune systems due to a variety of factors. Non-limiting examples of such factors comprise cancer, HIV, and organ transplants. Of the 68,000 cases of disseminated candidiasis, about 10-20% of the patients are severely immune suppressed.

Positive diagnosis of fungal infections, such as invasive Candida functions, is largely reliant on laboratory culturing of blood samples. However, blood cultures are often positive only late in the course of infection, leading to delayed diagnosis. Without wishing to be bound by theory, non-culture-based laboratory techniques can contribute to early diagnosis and management of invasive candidiasis. For example, both serologic (mannan, antimannan, and betaglucan) and molecular (Candida-specific PCR in blood and serum) based techniques have been applied as serial screening procedures in high-risk patients.

Identification of patients susceptible to fungal infections who can benefit from empirical antifungal treatment remains challenging, but it is necessary to avoid antifungal overuse in critically ill patients.

For non-neutropenic patients (i.e., patients with a functioning immune system) a Fluconazole loading dose of 800 mg (12 mg/kg) followed by a daily dose of 400 mg (6 mg/kg) Fluconazole is recommended for mild to moderate illness.

Echinocandin therapy (Caspofungin: loading dose of 70 mg, then 50 mg daily; micafungin: 100 mg daily; anidulafungin: loading dose of 200 mg, then 100 mg daily) is recommended for non-neutropenic adults with moderately severe to severe illness, or infection due to Candida glabrata.

For non-neutropenic patients, Amphotericin B deoxycholate (AmB-d) administered at a dosage of 0.5-1.0 mg/kg daily or a lipid formulation of AmB (LFAmB) administered at a dosage of 3-5 mg/kg daily are alternatives if there is intolerance to or limited availability of other antifungals.

For neutropenic patients (i.e., patients with an impaired immune system) an echinocandin (caspofungin: loading dose of 70 mg, then 50 mg daily; micafungin: 100 mg daily; anidulafungin: loading dose of 200 mg, then 100 mg daily) or LFAmB (3-5 mg/kg daily) is recommended for most patients, including those who are critically ill.

For neutropenic patients who are less critically ill and who have no recent azole exposure, fluconazole (loading dose of 800 mg [12 mg/kg], then 400 mg [6 mg/kg] daily) is a reasonable second-line treatment.

Voriconazole can be used in situations in which additional mold coverage is desired.

The recommended duration of therapy for candidemia without obvious metastatic complications is for 2 weeks after documented clearance of Candida from the bloodstream and resolution of symptoms attributable to candidemia.

Because mortality remains high in patients with candidemia, antifungal prophylaxis has been considered as a means to prevent its occurrence. However, there remain few, if any, effective options for antifungal prophylaxis, although broad use of Fluconazole has shown to decrease invasive candidiasis in some trials.

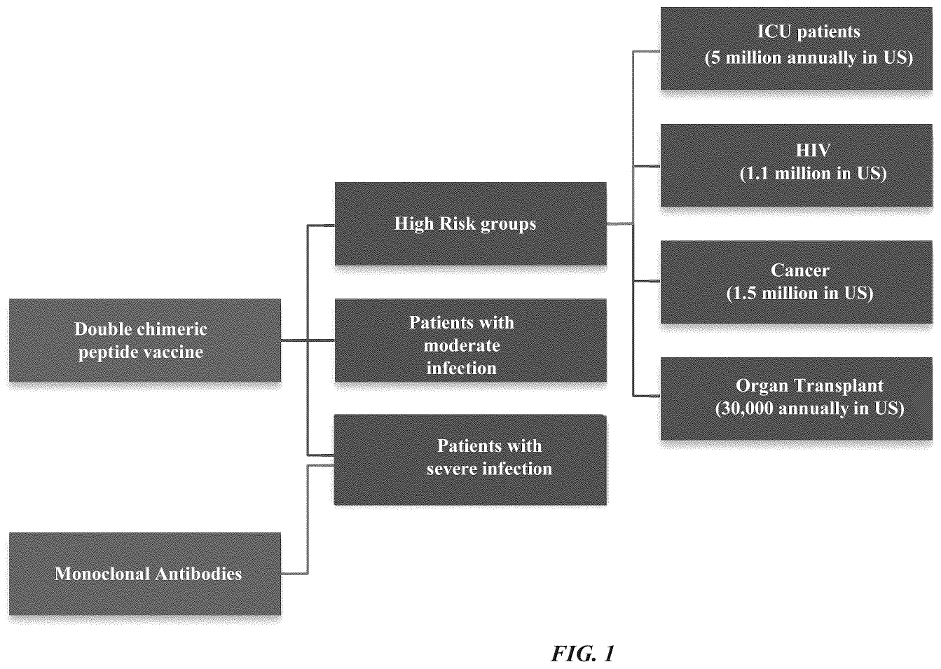

Examples of populations which can benefit from embodiments of the invention, such as anti-fungal vaccine and monoclonal antibodies, is summarized in FIG. 1. For example, a broad population can benefit from being administered a chimeric peptide vaccine or a composition comprising the same. As another example, subjects resistant to anti-fungal drugs or immune-suppressed subjects can benefit from administration of the mAbs.

### Peptide Vaccine

“Peptide vaccine” can refer to a peptide, for example those antigenic universal peptides described herein, or modified peptides thereof that is administered to a subject as a vaccine, either alone or as a component of a composition. For example, the peptide vaccine can comprise one or more antigenic universal peptides which share amino acid sequence homology across two or more different microorganisms and that can elicit an immune response so as to protect against infections. Antigenic universal peptides can be at least 3, 4, 5, 6, 7, 8, 9, 10, 11, 12, 13, 14, 15, 16, 17, 18, 19, 20, 21, 22, 23, 24, 25 or greater than 25 amino acids in length. In embodiments, the peptide vaccine comprises at least one of the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 79, SEQ ID NO: 26, SEQ ID NO: 27, SEQ ID NO: 28, SEQ ID NO: 81, or SEQ ID NO: 82.

“Chimeric peptide” or “chimeric peptide vaccine” refers to peptide comprising two or more peptides, such as the universal antigenic universal peptides as described herein, covalently linked to each other. In embodiments, the chimeric peptide vaccine comprises at least one of the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 79, SEQ ID NO: 26, SEQ ID NO: 27, SEQ ID NO: 28, SEQ ID NO: 81, or SEQ ID NO: 82.

The peptide vaccine can be a synthetic peptide vaccine. “Synthetic peptide vaccine” can refer to a peptide comprising the component of immunogenic determinants, i.e. a vaccine prepared by synthesizing a peptide according to the amino acid sequence of proteins, such as those proteins and sequences identified herein. For example, the synthetic peptide vaccine can be prepared by synthesizing a peptide of Fba, Met6, Hwp1, Enol1, Gap1, or Pgk1, and can share sequence homolog across 2 or more microorganisms. For example, the synthetic peptide vaccine can comprise the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 79, SEQ ID NO: 26, SEQ ID NO: 27, SEQ ID NO: 28, SEQ ID NO: 81, or SEQ ID NO: 82. In embodiments, the synthetic peptide vaccine can optionally comprise additional components such as an adjuvant and/or carrier protein.

In embodiments, the antigenic universal peptide comprises a peptide that is at least 80% identical in two or more different microorganisms. In other embodiments, the antigenic universal peptide comprises a peptide that is about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical in two or more microorganisms. In embodiments, the antigenic peptide comprises a peptide that is 100% identical in two or more microorganisms. For example, such organisms can comprise fungi, such as those relevant to disease (for example, Candida spp.), bacteria, viruses, or protozoa. Without wishing to be bound by theory, efficacy, such as protective efficacy, of the peptide vaccine correlates with the homology of the amino acid sequence between the two or more microorganisms. For example, protective efficacy is higher for a peptide that shares 100% homology in two or more microorganisms than that of a peptide that is 80% homologous.

For examples, embodiments comprise the antigenic universal peptide GPV-MO1 (SEQ ID NO. 81; PTTTIGSFPQ) whose amino acid sequence has 100% homology in medically important fungal species, such as Candida spp. Aspergillus Fumigatus, Aspergillus Niger, & Streptococcus agalactiae, Streptococcus suis.

As another example, embodiments can comprise the antigenic universal peptide GPV-MO1A (SEQ ID NO. 82; NLPLFPTTTIGSFPQTK), which corresponds to the peptide exposed on the surface of Candida albicans, and also shares 100% homology in medically important fungal species, such as Candida spp. In addition to C. albicans, Aspergillus Fumigatus & Aspergillus Niger.

Embodiments as described herein can elicit an immune response in a subject. As used herein, an “immunological response” or “immune response” to a peptide, for example an antigenic universal peptides as described herein or nucleic acid encoding the antigenic universal peptide, or composition comprising a polypeptide or nucleic acid encoding the polypeptide includes the development in a mammal of a cellular immune response that recognizes the polypeptide of the invention. In some examples, the immune response is a humoral immune response. In some examples, the cellular immune response additionally includes a humoral immune response. The immune response can be specific to the antigenic universal peptide, but this is not required. The immune response that is elicited by administration of the antigenic universal peptide, or nucleic acid encoding the antigenic polypeptide, can be any detectable increase in any facet of the immune response (e.g., cellular response, humoral response, cytokine production), as compared to the immune response in the absence of the administration of the polypeptide or nucleic acid.

Encompassed within the present invention are compositions in association with an antigenic universal peptide, or nucleic acid encoding the polypeptide that elicit the immune response.

As used herein, an “humoral immune response” can refer to an immune response mediated by antibody molecules or immunoglobulins. Antibody molecules of the present invention can include the classes of IgG (as well as subtypes IgG1, IgG2a, and IgG2b), IgM, IgA, IgD, and IgE. Antibodies functionally can include antibodies of a primary immune response as well as memory antibody responses or serum neutralizing antibodies. With respect to infectious disease, antibodies of the present invention can serve to, but are not required to, neutralize or reduce infectivity of the pathogen, and/or mediate antibody-complement, or antibody dependent cell cytotoxicity (ADCC) to the peptide. For more detailed information, see Peptide-Based Immunotherapeutics and Vaccines, Journal of Immunology Research Volume 2014 (2014), which is incorporated by reference herein in its entirety.

As used herein, a “cellular immune response” is one mediated by T-lymphocytes and/or other white blood cells, including without limitation natural killer (NK) cells and macrophages. T-lymphocytes of the present invention include T-cells expressing alpha/beta T-cell receptor subunits or gamma/delta receptor expressing T-cells and may be either effector or suppressor T-cells.

Embodiments as described herein can protect against fungal infections caused by yeast cells, or other microbial infections, such as those caused by bacteria, protozoa, and/or viruses. The outermost layer of the Candida yeast cell envelope is the cell wall. The cell wall maintains the structure and the rigidity of the cell but is freely permeable to solutes. Inside the cell wall, the plasma membrane forms a relatively impermeable barrier for hydrophilic molecules. In embodiments, the antigenic universal peptide comprises cell wall peptides, including, for example, fungal cell wall peptides and bacterial cell wall peptides.

In embodiments, the antigenic universal peptide comprises an amino acid sequence, or is encoded by a nucleic acid encoding the amino acid sequence (such as that obtained by reverse translation), corresponding to that of fungal cell wall proteins. Non-limiting examples of fungal cell wall proteins comprise fructose-bisphosphate aldolase (Fba; GenBank Accession No. AOW28947; Protein Accession No. XP_722690), methyltetrahydropteroyltriglutamate homocysteine methyltransferase (Met6; GenBank Access No. AOW30921; Protein Accession No. XP_718219), hyphal wall protein-1 (Hwp1; GenBank Access No. ACN63125), enolase (Enol; GenBank Accession No. AAA71939), glyceraldehyde-3-phosphate dehydrogenase (Gap1; GenBank Accession No. AOW29704), and phosphoglycerate kinase (Pgk1; GenBank Accession No. AAA66523) as described in Pitarch et al, [Pitarch A, Abian J, Carrascal M, Sanchez M, Nombela C, Gil C (2004) Proteomics-based identification of novel Candida albicans antigens for diagnosis of systemic candidiasis in patients with underlying hematological malignancies. Proteomics 4:3084-3106], Clancy et al [Clancy CJ, Nguyen M-L, Cheng S, Huang H, Fan G, Jaber RA, Wingard JR, Cline C, Nguyen MH (2007) Immunoglobulin G responses against a panel of Candida albicans antigens as accurate and early markers for the presence of systemic candidiasis. J Cin Microbiol 46:1647-1654], and Pitarch et al, [Prediction of the clinical outcome in invasive candidiasis patients based on molecular fingerprints of five anti-Candida antibodies in serum. Mol Cell Proteomics. 2011 January; 10(1):M110.004010. doi: 10.1074/mcp.M110.004010. Epub 2010 Sep. 21], each of which are hereby incorporated by reference in their entireties.

In embodiments, the antigenic universal peptide comprises an amino acid sequence, or is encoded by a nucleic acid encoding the amino acid sequence (such as that obtained by reverse translation), corresponding to that of Fba Protein Accession No. XP_722690; 359 amino acids (SEQ ID NO: 53):

| | |
|---|---|
| 1 | mappavlsks gviygkdvkd lfdyaqekgf aipainvtss stvvaaleaa rdnkapiilq |
| 61 | tsqggaayfa gkgvdnkdqa asiagsiaaa hyiraiapty gipvvlhtdh cakkllpwfd |
| 121 | gmlkadeeff aktgtplfss hmldlseetd deniatcaky fermakmgqw lemeigitgg |
| 181 | eedgvnnehv ekdalytspe tvfavyeslh kispnfsiaa afgnvhgvyk pgnvqlrpei |
| 241 | lgdhqvyakk qigtdakhpl ylvfhggsgs tqeefntaik ngvvkvnldt dcqyayltgi |
| 301 | rdyvtnkiey lkapvgnpeg adkpnkkyfd prvwvregek tmskriaeal difhtkgql |

In embodiments, the antigenic universal peptide comprises an amino acid sequence, or is encoded by a nucleic acid encoding the amino acid sequence (such as that obtained by reverse translation), corresponding to that of Met6 Protein Accession No. XP_718219; 767 amino acids (SEQ ID NO: 54):

| | |
|---|---|
| 1 | mvqssvlgfp riggqrelkk iteaywsgka tveellakgk elrehnwklq qkagvdiips |
| 61 | ndfsyydqvl dlsllfnaip erytkfdlap idvlfamgrg lqkkatetqa avdvtalemv |
| 121 | kwfdsnyhyv rptfshstef klntaagikp vdefneakal gvqtrpvilg pvsylylgka |
| 181 | dkdsldlepi sllpkilpvy kellqklkea gaeqvqidep vlvldlpeav qskfkeayda |
| 241 | lvgadvpeli lttyfgdvrp nlkaienlpv agfhfdfvrv peqldevasi lkdgqtlsag |
| 301 | vvdgrniwkt dfakasavvq kaiekvgkdk vvvatsssll htpvdleset kldavikdwf |
| 361 | sfatqkldev vviaknvsge dvskqleana asikarsess itndpkvqer lttinealat |
| 421 | rkaafperlt eqkakynlpl fptttigsfp qtkdirinrn kfakgqitae eyeafinkei |
| 481 | etvvrfqeei gldvlvhgep erndmvqyfg eqlngfaftt ngwvqsygsr yvrppiivgd |
| 541 | vsrpkamtvk esvyaqsits kpmkgmltgp vtilrwsfpr ddvsgkiqal qlglalrdev |
| 601 | ndlegagitv iqvdepaire glplragker sdylnwaaqs frvatsgven stqihshfcy |
| 661 | sdldpnhika ldadvvsief skkddpnyiq efseypnhig lglfdihspr ipskqefvsr |
| 721 | ieeilkvypa skfwvnpdcg lktrgwpevk esltnmveaa kefraky |

In embodiments, the antigenic universal peptide comprises an amino acid sequence, or is encoded by a nucleic acid encoding the amino acid sequence (such as that obtained by reverse translation), corresponding to that of Hwp1 Accession No. ACN63125; 264 amino acids (SEQ ID No: 55)

| | |
|---|---|
| 1 | sssapattpn tsvpttttet kssstplttt tehdttvvtv tscsnsvcte sevttgvivi |
| 61 | tskdtiytty cpltettpvs tapatetptg tvststeqst tvitvtscse ssctesevtt |
| 121 | gvvvvtseet vyttfcplte ntpgtdstpe asippmetip agsqpsipag etspavpksd |
| 181 | vpatesapap emtpagtetk paapkssapa tepspvapgt esapagpgas sspkssvlas |
| 241 | etspiapgae tapagssgai tipe |

In embodiments, the antigenic universal peptide comprises an amino acid sequence, or is encoded by a nucleic acid encoding the amino acid sequence (such as that obtained by reverse translation), corresponding to that of Enolase Accession No. AAA71939; 440 amino acids (SEQ ID NO. 56)

| | |
|---|---|
| 1 | msyatkihar yvydsrgnpt vevdfttdkg lfrsivpsga stgvhealel rdgdkskwlg |
| 61 | kgvlkavanv ndiiapalik akidvvdqak idefllsldg tpnksklgan ailgvslaaa |
| 121 | naaaaaqgip lykhianisn akkgkfvlpv pfqnvlnggs haggalafqe fmiaptgvst |
| 181 | fsealrigse vyhnlksltk kkygqsagnv gdeggvapdi ktpkealdli mdaidkagyk |
| 241 | gkvgiamdva ssefykdgky dldfknpesd pskwlsgpql adlyeqlise ypivsiedpf |
| 301 | aeddwdawvh ffervgdkiq ivgddltvtn ptriktaiek kaanalllkv nqigtltesi |
| 361 | qaandsyaag wgvmvshrsg etedtfiadl svglrsgqik tgaparserl aklnqilrie |
| 421 | eelgseaiya gkdfqkasql |

In embodiments, the antigenic universal peptide comprises an amino acid sequence, or is encoded by a nucleic acid encoding the amino acid sequence (such as that obtained by reverse translation), corresponding to that of Gap1 Accession No. AOW29704; 582 amino acids (SEQ ID NO: 57)

| | |
|---|---|
| 1 | mlhkketndt fvqlnrspst geqkssgiws sikdsfkpal pqdkltgvdd ipdreltdie |
| 61 | rininaansn lqrklktrhl qmiaigssig tglfvgtgga lstggpaaiv lawaisaisv |
| 121 | fmtmqglgel avafpvsggf nlyaskflep gigfavgwny flqffvllpl elvagaitik |
| 181 | ywnasinsdv fviifwfvvl vitmlgvrwy geaelvfcti kviavigfii lgivlicggg |
| 241 | pnhefiggky wrepgpfans fkgfasslit aafsfggtem ialtasessn vrhalpkaik |
| 301 | qvfwrivify lgsiimiatl vpyndkrllg sssvdvtasp ftiaivnggi kglpsvinav |
| 361 | ilisvlsvgn asvyatsrtl nslaeqgmap kwtgyidrag rplfailitn vfglfaliaa |
| 421 | dnekqvvafn wllalsglss iftwmsinls hirfrramkv qnrsltelpf vaqsgvwgsy |
| 481 | fgltlnilyl iaqfyiglfp vggkpnaydf flaylgvpvi laswigykiw krdwtlfira |
| 541 | kdidldtgri nvdldllqqe iaeekaqlae kpfyiriyrf wc |

In embodiments, the antigenic universal peptide comprises an amino acid sequence, or is encoded by a nucleic acid encoding the amino acid sequence (such as that obtained by reverse translation), corresponding to that of Pgk1 Accession No. AAA66523; 417 amino acids (SEQ ID NO: 58)

| | |
|---|---|
| 1 | mslsnklsvk dldvagkrvf irvdfnvpld gktitnnqri vaalptikyv eehkpkyivl |
| 61 | ashlgrpnge rndkyslapv atelekllgq kvtflndcvg pevtkavena kdgeifllen |
| 121 | lryhieeegs skdkdgkkvk adpeavkkfr qeltsladvy indafgtahr ahssmvglev |
| 181 | pqraagflms keleyfakal enperpflai lggakvsdki qlidnlldkv dmlivgggma |
| 241 | ftfkkilnkm pigdslfdea gaknvehlve kakknnveli lpvdfvtadk fdkdaktssa |
| 301 | tdaegipdnw mgldcgpksv elfqqavaka ktivwngppg vfefekfangtkslldaavk |
| 361 | saengnivii gggdtatvak kygvveklsh vstgggasle llegkdlpgv valsnkn |

In embodiments, the antigenic universal peptide comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 79, SEQ ID NO: 26, SEQ ID NO: 27, SEQ ID NO: 28, SEQ ID NO: 81, or SEQ ID NO: 82. In some embodiments, the antigenic universal peptide comprises at least 3, 4, 5, 6, 7, 8, consecutive amino acids of any one of SEQ ID NOS: 1-28. In some embodiments, the antigenic universal peptide comprises at least 60%, 65%, 70%, 75%, 80%, 85%, 90%, 91%, 92% identity to any one of SEQ ID NOS: 1-28, 81-82.

Embodiments as described herein can further comprise multiple antigenic universal peptides covalently linked to each other, such as two or more antigenic universal peptides covalently linked to each other. For example, embodiments can comprise 2, 3, 4, 5, 6, 7, 8, 9, 10, 11, or 12 antigenic universal peptides covalently linked. One or more of the linked antigenic universal peptides can be at least 80% identical in two or more different microorganisms.

Examples of antigenic peptides covalently linked to each other comprise SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, or SEQ ID NO: 93.

Embodiments as described herein can comprise peptides linked in different orders and orientations. For example, the linked construct can comprise NH₃-PEPTIDE1-Linker-PEPTIDE2-COOH or NH₃-PEPTIDE2-Linker-PEPTIDE1-COOH.

In certain embodiments, the order of the linked peptides may not be relevant to the protective efficacy of the peptide vaccine. For example, the covalently linked universal peptide can comprise NH₃-(SEQ ID NO: 1)-KK-(SEQ ID NO: 14)-COOH (SEQ ID NO: 272) or NH₃-(SEQ ID NO: 14)-KK-(SEQ ID NO. 1)-COOH (SEQ ID NO: 273), each of which having similar protective efficacy.

In other embodiments, the order of the linked peptides is important for the protective efficacy of the vaccine, such as is the case when one construct protects against infection more effectively than another construct. For example, NH₃-PEP1-KK-PEP2-COOH works better than NH₃-PEP2-KK-PEP1-COOH, even though NH₃-PEP2-KK-PEP1-COOH itself can induce moderate protection (˜40%-50% survival).

In embodiments, the antigenic universal peptides are covalently linked by a linker, such as a peptide linker. Non-limiting examples of such peptide linkers comprise the amino acid sequence KK, GPSL (SEQ ID NO: 50), a universal CD4 T cell helper peptide, also known as non-natural pan DR epitope or PADRE (SEQ ID NO: 97): aKXVAAWTLKAAaZC (X=1-cyclohexylalanine, Z=aminocaproic acid). In embodiments, the linker is a helical linker (such as EAAAK; SEQ ID NO: 76), wherein in other embodiments the linker is a flexible linker (such as GGGGS; SEQ ID NO: 77). Examples of antigenic peptides covalently linked to each other comprise SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, or SEQ ID NO: 93.

EAAAK (SEQ ID NO: 76) is an alpha helix-forming linker which can be used as a linker in fusion proteins. The α-helical structure is rigid and stable, with intra-segment hydrogen bonds and a closely packed backbone. Thus, stiff α-helical linkers, such as EAAAK (SEQ ID NO: 76), can act as rigid spacers between protein domains, maintaining the structure of the epitope intact, especially binding sites. Also, EAAAK motif (SEQ ID NO: 76) can effectively separate functional domains.

GGGGS linker (SEQ ID NO: 77) is a flexible linker (GGGGS)ₙ(SEQ ID NO: 77) (Huston J et al. Protein engineering of antibody binding sites: recovery of specific activity in an anti-digoxin single-chain Fv analogue produced in Escherichia coli. Proc Natl Acad Sci USA. 1988; 85:5879-5883, incorporated herein by reference in its entirety) that can be used to construct an antibody fragment, such as scFv, since its flexible structure allows for the correct orientation of the V_{H} and V_{L} domains and does not interfere with the folding of the protein domains.

In certain embodiments, the peptide comprises two or more linkers, further including combinations of helical and flexible linkers. For example, embodiments can comprise both RGD and KK in chimeric-peptide vaccines, such as those comprise an Fba peptide and a Met6 peptide, such as RGD-(SEQ ID NO: 26)-KK-(SEQ ID NO: 1)-KLH (SEQ ID NO: 274).

GPSL (SEQ ID NO: 50) is a 4 amino acid linker that functions as a flexible linker joining the B cell and the T cell epitopes of the vaccine without transporting structural information to the other part. Without wishing to be bound by theory, the GPSL unit (SEQ ID NO: 50) connects the two or more parts of the peptide vaccine without transferring any structural information from one part to the other. For example, see Structural Characterization by NMR of a Double Phosphorylated Chimeric Peptide Vaccine for Treatment of Alzheimer's Disease Molecules 2013, 18, 4929-4941, which is incorporated by reference herein in its entirety.

Embodiments as described herein can comprise universal T cell epitope, such as TT830-844 (QYIKANSKFIGITE; SEQ ID NO: 75) or TT947-967 (FNNFTVSFWLRVPKVSASHLE; SEQ ID NO: 78).

The production of antibodies directed to and/or that recognize epitopes in the antigenic universal peptides of the invention, such as those described herein, can be increased significantly by introducing a di-lysine spacer (KK). Cathepsin B, one of the important proteases for antigen processing in the context of MHC class II molecules, digests the di-lysine amino acid sequence. Without wishing to be bound by theory, the reduction in antibody production to the newly generated epitope by tandem repeating of peptides is related to the digestion of the spacer by the protease. Peptides with an RGD motif at the N-terminal side of the -KK- linker strongly induced the antibodies to the peptides derived from the C-terminus of the KK linker of the antigenic universal peptide. However, only minimal levels of peptide antibodies were induced when the determinant was on the N-terminal side of the KK linker. The KK linker greatly reduced the induction of the antibodies that could recognize the KK including regions. (RGD motif enhances immunogenicity and adjuvanticity of peptide antigens following intranasal immunization; Vaccine 22 (2003) 237-243, which is incorporated by reference herein in its entirety).

The insertion of two lysine residues at the boundary of a B-cell epitope and a class II presentable peptide (T-cell epitope) enhances the antibody induction capacity of this type of peptide construct. Pairs of basic residues such as KK, KR and RR appeared to be the main cleavage site of cathepsin B, one of the most important enzymes for antigen processing in the context of class II molecules.

RGD enhances the immunogenicity of peptide antigens by addition of motifs that bind to cell attachment proteins, such as arginine-glysine-aspartate (RGD), to the amino acid sequence. RGD, an integrin-binding motif, is the strongest, among several molecules reported, giving an average of 10 times enhancement of antibody titers when incorporated into several peptide antigens. (RGD motif enhances immunogenicity and adjuvanticity of peptide antigens following intranasal immunization; Vaccine 22 (2003) 237-243, which is incorporated herein by reference in its entirety).

The pan HLA DR-binding epitope (PADRE; SEQ ID NO: 51) is a simple carrier epitope suitable for use in the development of synthetic and recombinant vaccines. Since PADRE is capable of binding to murine I-A^{b} molecules, it was possible to evaluate the in vivo immunogenicity of simple linear constructs using PADRE as a T helper epitope in conjunction with various Candida B cell peptide epitopes. This epitope is a synthetic, non-natural pan HLA DR-binding Epitope (PADRE) peptide that binds with high or intermediate affinity to 15 of 16 of the most common HLA-DR types tested to date. (Linear PADRE T Helper Epitope and Carbohydrate B Cell Epitope Conjugates Induce Specific High Titer IgG Antibody Responses; The Journal of Immunology, 2000, 164: 1625-163, which is incorporated by reference herein in its entirety).

In embodiments, an antigenic universal peptide of the invention can comprise a polypeptide comprising at least 2 or more covalently linked antigenic universal peptides, wherein each antigenic universal peptide is at least 80% identical in two or more different microorganisms. For example, the antigenic universal peptides can be at least 3, 4, 5, 6, 7, 8, 9, 10, 11, 12, 13, 14, 15, 16, 17, 18, 19, 20, 21, 22, 23, 24, 25 or greater than 25 amino acids in length comprising peptides corresponding to fructose-bisphosphate aldolase (Fba), methyltetrahydropteroyltriglutamate homocysteine methyltransferase (Met6), hyphal wall protein-1 (Hwp1), enolase (Enol), glyceraldehyde-3-phosphate dehydrogenase (Gap1), and phosphoglycerate kinase (Pgk1).

In embodiments, chimeric peptides, such as those comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 79, SEQ ID NO: 26, SEQ ID NO: 27, SEQ ID NO: 28, SEQ ID NO: 81, or SEQ ID NO: 82 comprises a helical linker (such as EAAAK; SEQ ID NO: 76), a flexible linker (such as GGGGS; SEQ ID NO: 77), and/or at least one covalent linker selected from the group consisting of KK, GPSL (SEQ ID NO: 50), a universal CD4 T cell helper peptide (also known as non-natural pan DR epitope (PADRE) (SEQ ID NO: 51).

In other embodiments, the chimeric peptide can comprise an antigenic universal peptides covalently linked to a peptide of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48. Such embodiments, for example, can comprise a helical linker (such as EAAAK; SEQ ID NO: 76), a flexible linker (such as GGGGS; SEQ ID NO: 77), and/or at least one covalent linker selected from the group consisting of KK, GPSL (SEQ ID NO: 50), a universal CD4 T cell helper peptide (also known as non-natural pan DR epitope (PADRE) (SEQ ID NO:51). For a discussion of peptides for fungal infections, see also U.S. Pat. No. 9,416,173, which is incorporated by reference herein in its entirety.

In embodiments, the antigenic universal peptide can be modified to be used as a vaccine in a mammal. For example, modification can include methylation, amidation, acetylation, PEGylation etc. Such modifications can be modifications of the N terminus, the C terminus, and/or internal to the peptide. Such modifications can be carried out onto one or more amino acids of an antigenic universal peptide of the invention. For example, modifications of the antigenic universal peptide can increase the peptide's stability in vivo, elicit a stronger immune response, or allow the peptide to imitate its natural structure.

In embodiments, the peptide modification can be methylation. The methylation of proteins, for example, can help regulate cellular functions such as transcription, cell division, and cell differentiation. Methylation of the antigenic universal peptides, for example, can extend the half-life of the peptides, and can also be used to identify the binding sites of peptides with antibodies. Methylation of amino acid residues can be performed according to methods well understood by one of ordinary skill in the art (see US20090264620 and Mini Rev Med Chem. 2016; 16(9):683-90, each of which are incorporated by reference herein in their entireties entirety).

Without being bound by theory, an important aspect of using a methylated peptide is the role this modification plays in the regulation of protein-protein interactions. The methylation of peptides can either inhibit or promote peptide-antibody interactions, depending on the type of methylation (Lee, Young-Ho, and Michael R. Stallcup. “Minireview: protein arginine methylation of nonhistone proteins in transcriptional regulation.” Molecular endocrinology 23.4 (2009): 425-433; Poornima, Gopalakrishna, et al. “Arginine methylation promotes translation repression activity of eIF4G-binding protein, Scd6.” Nucleic Acids Research (2016): gkw762; Bedford, Mark T., et al. “Arginine methylation inhibits the binding of proline-rich ligands to Src homology 3, but not WW, domains.” Journal of Biological Chemistry 275.21 (2000): 16030-16036, each of which are incorporated herein by reference in their entireties). In one embodiment, each amino acid residue of the universal peptide is methylated. In subsequent embodiments, these methylated peptides are tested for their binding affinity to the monoclonal antibodies specific for the original, unmethylated peptide antigen. Also, the specificity and binding affinity of methylated peptide to these mAbs are compared with that of the original, unmethylated, peptide in order to understand which amino acid is the key for the peptide and antibody binding.

In embodiments, the peptide modification can be amidation or acetylation, such as at the C terminus or N terminus, respectively. As the antigenic universal peptide can be that of an internal amino acid sequence of a protein, terminal amidation (such as that of the C-terminus) or acetylation (such as that of the N-terminus) of the peptide will remove the charge of the peptide and help it imitate its natural structure. In other words, the modified peptide termini are uncharged, allowing the modified peptide to more closely mimic that of the native protein. Such modifications can also increase the metabolic stability of the peptides, as well as their ability to resist enzymatic degradation by aminopeptidases, exopeptidases, and synthetases. Amidation and acetylation of amino acid residues can be performed according to methods well understood by the skilled artisan, for example see Cottingham, Ian R., et al. “A method for the amidation of recombinant peptides expressed as intein fusion proteins in Escherichia coli.” Nature biotechnology 19.10 (2001): 974-977, Cerovsky, Václav, and Maria-Regina Kula. “Peptide amidase-catalyzed C-terminal peptide amidation in a mixture of organic solvents.” Peptides for the New Millennium (2002): 142-143; Mura, Manuela, et al. “The effect of amidation on the behaviour of antimicrobial peptides.” European Biophysics Journal 45.3 (2016): 195-207; Thomas, A., Towards a Functional Understanding of Protein N-Terminal Acetylation. PLOS Biol. 2011, 9(5); and Wallace, R. J., Acetylation of peptides inhibits their degradation by rumen micro-organisms. British Journal of Nutrition. 1992, 68, 365-372, each of which are incorporated by reference in their entireties.

In embodiments, the peptide modification can be acetylation, for example N-terminal acetylation (see U.S. Pat. No. 9,062,093, which is incorporated herein by reference in its entirety). Peptides such as those described herein can be derived from either the N- or C-terminus of cell wall proteins, but are still considered internal peptides of the cell wall proteins and not necessarily at the extreme end of the N- or C-terminus of the proteins. When the peptide is from an internal sequence of a protein, terminal amidation (C-terminus) or acetylation (N-terminus) will remove its charge and help it imitate its natural structure (amide, CONH₂). In addition, this modification makes the resulting peptide more stable towards enzymatic degradation resulting from exopeptidases.

In embodiments, the peptide modification can be PEGylation, or linking of the antigenic universal peptides to polyethylene glycol, so as to increase solubility and prolong circulatory time, for example. Once linked to a peptide, the PEG subunit becomes tightly associated with two or three water molecules, which has the dual function of rendering the antigenic universal peptide more soluble in water and making its molecular structure larger. As the kidneys filter substances according to size, the addition of PEG's molecular weight can prevent the premature renal clearance undergone by small peptides. PEG's globular structure can also act as a shield to protect the antigenic universal peptide of the invention from proteolytic degradation, and can reduce the immunogenicity of foreign peptides by limiting their uptake through the dendritic cells. PEG itself is not immunogenic or toxic, and allows for lower doses and less-frequent administrations. In some instances, PEG can increase the circulating half-life of a peptide drug by more than 100 times. In addition to improving the pharmacokinetic and pharmacodynamic properties of peptide drugs once inside the body, PEGylation can also aid drug delivery because PEGylated peptides act as permeation enhancers for nasal drug delivery.

In embodiments, the PEG molecule can be monomethoxy PEG (mPEG), which has relatively simple chemistry due to its monofunctionality (CH₃O—(CH₂CH₂O)n-CH₂CH₂—OH). In other embodiments, the PEG molecule can be HiPEG, or PEG attached to histidine sequences expressed on the N or C terminal of proteins. For example, 6 His-tags (SEQ ID NO: 94) can be used to create site-specific PEGylated conjugates, that is, PEGylation using a His-tagging approach. A protein is encoded with a polyhistidine tag (such as a 6 histidine tag (SEQ ID NO: 94)). Once incubated with a Ni-nitrilotriacetic acid (NTA)-PEG reagent, a complex is formed between the histidine residues and the nickel ion, thus PEGylating the protein. In other embodiments, the PEG molecule can be branched or forked PEG, such as PEG2, releasable PEGs (rPEGs), or heterbifunctional PEGs, details of which can be found in Roberts, et al, which is incorporated by reference herein in its entirety (Roberts, M. J., M. D. Bentley, and J. M. Harris. “Chemistry for peptide and protein PEGylation.” Advanced drug delivery reviews 64 (2012): 116-127). One of ordinary skill in the art appreciates the routine methods practiced to pegylate amino acid residues of peptides of interest.

In embodiments, a cysteine residue can be incorporated onto the C-terminus and/or N-terminus of the antigenic universal peptide, allowing for conjugation of the peptide to other components, for example carrier proteins such as KLH, BSA, or TT. Methods of incorporating cysteine residues are well understood in the art (e.g., see Chapter 3 Peptide-carrier conjugation: Laboratory Techniques in Biochemistry and Molecular Biology; Volume 19, 1988, Pages 95-130).

In embodiments, the antigenic universal peptide can be conjugated to a macromolecule, non-limiting examples of which comprise carrier proteins such as keyhole limpet hemocyanin (KLH), tetanus toxoid (TT), or bovine serum albumin (BSA). Conjugation of the peptide to such molecules, for example, can increase the stability of the peptide, or can increase resistance to proteolytic cleavage. Conjugation methods as listed herein are well understood by the skilled artisan (Chapter 3 Peptide-carrier conjugation: Laboratory Techniques in Biochemistry and Molecular Biology; Volume 19, 1988, Pages 95-130).

“Conjugation” can refer to the linking of a peptide, either directly or indirectly, to another molecule. For example, “direct conjugation” can refer to linking of the antigenic universal peptide to an activated carbohydrate, another antigenic universal peptide, or a peptide linker, without introducing additional functional groups. As another example, “indirect conjugation” can refer to the addition of functional groups which are used to facilitate conjugation. For example, carbohydrate can be functionalized with amines which are subsequently reacted with bromoacetyl groups. The bromoacetylated carbohydrate is then reacted with thiolated protein. (Hermanson, GT, Bioconjugate Techniques, Academic Press, 2^{nd} ed, 2008). The term “functionalization” generally means to chemically attach a group to add functionality, for example, to facilitate conjugation. Examples include functionalization of proteins with hydrazides or aminooxy groups and functionalization of carbohydrate with amino groups.

In embodiments, the antigenic universal peptide of the invention can comprise a Multiple Antigenic universal peptide (MAP), which can allow for the production of high-titer anti-peptide antibodies (Fujita and Taguchi, Chemistry Central Journal 2011, 5:48 Proc. Natl. Acad. Sci. USA Vol. 85, pp. 5409-5413, August 1988, which is incorporated by reference herein in its entirety). Multiple antigen peptide application can be used to produce high-titer anti-peptide antibodies and synthetic peptide vaccines. This system utilizes the α- and F-amino groups of lysine to form a backbone to which multiple peptide chains can be attached. Depending on the number of lysine tiers, different numbers of peptide branches can be synthesized. This eliminates the need to conjugate the antigen to a protein carrier. MAP is a branched peptide at which linear peptide chains are linked at their C-terminus via polylysine core, thereby increasing the size of whole molecule. For example, 4MAP-peptide refers to four branches of peptide sequences with 3 lysine cores. For the synthesis of MAPs and MAP-based systems, please refer to Kowalczyk, Wioleta, et al. “Synthesis of multiple antigenic peptides (MAPs)—strategies and limitations.” Journal of Peptide Science 17.4 (2011): 247-251; Tam, James P. “Synthetic peptide vaccine design: synthesis and properties of a high-density multiple antigenic peptide system.” Proceedings of the National Academy of Sciences 85.15 (1988): 5409-5413; Fujita, Yoshio, and Hiroaki Taguchi. “Current status of multiple antigen-presenting peptide vaccine systems: Application of organic and inorganic nanoparticles.” Chemistry Central Journal 5.1 (2011): 48, each of which are incorporated herein by reference in their entireties.

Multiple Antigenic universal peptides (MAPs) are peptides that are branched artificially, in which Lys residues are used as the scaffolding core to support the formation branches, for example 1, 2, 3, 4, 5, 6, 7, 8, 9, 10 or greater than 10 branches, with varying peptide sequences or the same peptide sequences. Multiple Antigen Peptides have been used

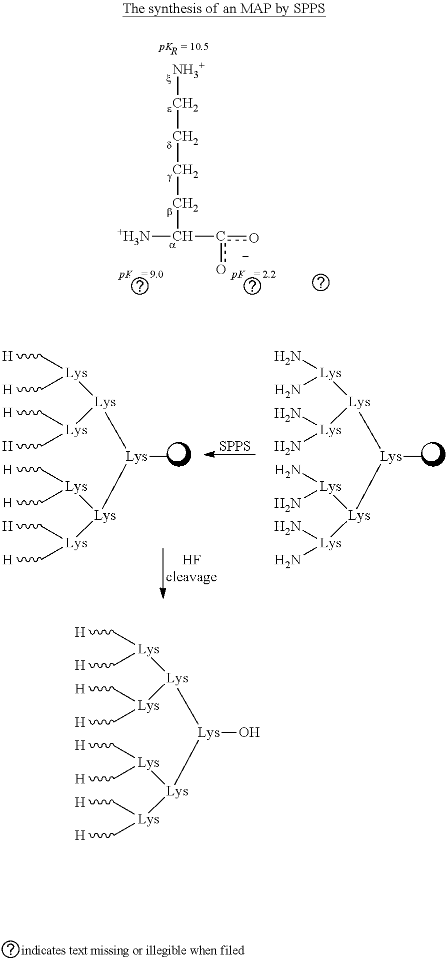

to produce antibodies for use in immunological studies. MAPs have a high molar ratio of the peptide antigen to the core molecule, and no carrier protein is needed to elicit an antibody response.

Standard SPSS can be used to synthesize MAPs. This process involves anchoring Boc-Lys(Boc)-OH to a resin, followed by the sequential treatment with TFA, deprotection, coupling, and deprotection cycles. The peptides to be used in immunological studies are then synthesized on each branch. For the synthesis of MAPs and MAP-based systems, please refer to Kowalczyk, Wioleta, et al. “Synthesis of multiple antigenic peptides (MAPs)-strategies and limitations.” Journal of Peptide Science 17.4 (2011): 247-251; Tam, James P. “Synthetic peptide vaccine design: synthesis and properties of a high-density multiple antigenic peptide system.” Proceedings of the National Academy of Sciences 85.15 (1988): 5409-5413; Fujita, Yoshio, and Hiroaki Taguchi. “Current status of multiple antigen-presenting peptide vaccine systems: Application of organic and inorganic nanoparticles.” Chemistry Central Journal 5.1 (2011): 48, each of which are incorporated herein by reference in their entireties.

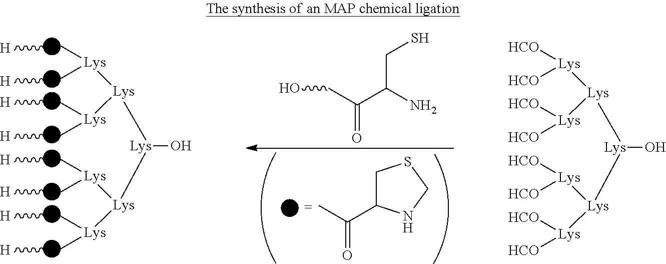

There are no known branched proteins in nature. However, the synthesis of MAP can be problematic. The strict spacing between each of the eight branches might lead to aggregation of the peptides on the resin, and could result in low coupling yields and peptide deletions. The PeptideSyn technology used by LifeTein (https://www.lifetein.com/multiple-antigenic-peptides.html), for example, overcomes this problem using a chemical ligation strategy, which allows production of the desired peptide dendrimer at an increased yield. The branched structures can then form an increased molecular weight protein to elicit an immunogenic response.

MAP peptides perform like large proteins as four or eight copies of antigenic universal peptides of interest, for example, are synthesized on a branched lysine.

MAP peptides comprising the antigenic universal peptides of the invention can be injected directly with an adjuvant for antibody production. A carrier protein is not required.

The MAP technique favors N-terminal or internal peptides because the peptide is linked through the C-terminus. In contrast, KLH-conjugation favors C-terminal peptides. However, the KLH carrier might cause steric hindrance.

Without wishing to be bound by theory, MAP peptides can result in a slightly higher titer than KLH-conjugated peptides due to their structure. The MAP design maximizes the antigen concentration because the synthesized peptide-antigen accounts for up to 95% of the total weight of the final product.

With MAPs, a known amount of peptide can be used for immunization each time, giving greater control over the experimental conditions.

Embodiments as described herein further comprise methods for selecting a peptide vaccine directed to microbial infections. For example, one embodiment comprises aligning the amino acid sequences of two or more proteins, such as cell wall proteins as described herein, and selecting or identifying a peptide sequence, such as those of at least 7-9 consecutive amino acids, wherein the amino acid sequence is at least 70% identical in the two or more cell surface proteins.

Embodiments further comprise the steps of analyzing the hydrophilicity of each amino acid and of the peptide, as well as the score of antigenicity. Hydrophilic peptides tend to appear on the cell surface, which is the first contacting immune cells. Hydrophilic peptides can be the target for vaccine candidates as they score higher for their antigenicity.

### Vaccine Composition

The term “vaccine” refers to a composition or compound (e.g., an antigen) used to stimulate an immune response in a mammal and so confer resistance to the disease or infection in that mammal, including an ability of the immune system to remember the previously encountered antigen. Antibodies are produced as a result of the first exposure to an antigen and stored in the event of subsequent exposure.

Embodiments as described herein are directed to a vaccine composition comprising at least one antigenic universal peptide comprising any one of the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 79, SEQ ID NO: 26, SEQ ID NO: 27, SEQ ID NO: 28, SEQ ID NO: 81, or SEQ ID NO: 82 or a combination thereof, and a pharmaceutically acceptable carrier. In some embodiments, the vaccine composition comprises at least one antigenic universal peptide encoded by a nucleic acid, such as a nucleic acid encoding for any one of the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 79, SEQ ID NO: 26, SEQ ID NO: 27, SEQ ID NO: 28, SEQ ID NO: 81, or SEQ ID NO: 82 and a pharmaceutically acceptable carrier. In some embodiments, the vaccine composition comprises at least one antigenic universal peptide encoded by a nucleic acid harbored by vector for expression of the same, and a pharmaceutically acceptable carrier. The term “pharmaceutically acceptable carrier” can refer to a one or more compatible solid or liquid filler diluents or encapsulating substances which are suitable for administration to a mammal, preferably a human. Typically, the carrier may be a solid, liquid, solution, suspension, gel, ointment, lotion, or combinations thereof as further discussed herein.

In embodiments, the vaccine composition can comprise two or more antigenic universal peptides of the invention, such as two or more antigenic universal peptides as described herein. Embodiments can comprise 1, 2, 3, 4, 5, 6, 7, 8, 9, 10, 11, or 12 antigenic universal peptides, including those as described herein, non-limiting examples of which comprise the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 79, SEQ ID NO: 26, SEQ ID NO: 27, SEQ ID NO: 28, SEQ ID NO: 81, or SEQ ID NO: 82.

In other embodiments, the vaccine composition can further comprise a peptide of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48.

“Adjuvant” can refer to a pharmacological or immunological agent that can be added to a vaccine composition to modify the immune response by boosting it such as to give a higher amount of antibodies and longer-lasting protection, thus minimizing the amount of foreign material to be administered to a subject. Adjuvants can also be used to enhance the efficacy of a vaccine by helping to modify the immune response to particular types of immune system cells: for example, by activating T cells instead of antibody-secreting B cells depending on the purpose of the vaccine. Adjuvants can also be used in the production of antibodies from immunized animals. There are different classes of adjuvants that can push immune response in different directions, but the most commonly used adjuvants include aluminum hydroxide and paraffin oil. Examples of adjuvants include, but are not limited to, helper peptide; aluminum salts such as aluminum hydroxide gel (alum) or aluminum phosphate; Freund's Incomplete Adjuvant and Complete Adjuvant (Difco Laboratories, Detroit, Mich.); Merck Adjuvant 65 (Merck and Company, Inc., Rahway, N.J.); Ribi-R730; Adjuplex (Wegmann et al, Clin Vaccine Immunol. 2015 September; 22(9):1004-12, incorporated herein by reference in its entirety); AS-2 (Smith-Kline Beecham); QS-21 (Aquilla); MPL or 3d-MPL (Corixa Corporation, Hamilton, Mont.); LEIF; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A; muramyl tripeptide phosphatidyl ethanolamine or an immunostimulating complex, including cytokines (e.g., GM-CSF or interleukin-2, -7 or -12) and immunostimulatory DNA sequences. In some embodiments, such as with the use of a polynucleotide vaccine, an adjuvant such as a helper peptide or cytokine can be provided via a polynucleotide encoding the adjuvant.

In embodiments, the vaccine composition can optionally comprise an adjuvant, whereas in other embodiments the vaccine composition does not comprise an adjuvant. Peptide vaccines without need for an adjuvant can maintain their protective efficacy while limiting the adverse effects that are associated with adjuvants, such as aluminum based adjuvants. For example, aluminum adjuvants can induce serious immunological disorders in humans, such as risk for autoimmunity, long-term brain inflammation and associated neurological complications, and can have profound and widespread adverse health consequences (Tomljenovic, L., and C. A Shaw. “Aluminum vaccine adjuvants: are they safe?.” Current medicinal chemistry 18.17 (2011): 2630-2637 and Petrovsky, Nikolai, and Julio Cesar Aguilar. “Vaccine adjuvants: current state and future trends.” Immunology and cell biology 82.5 (2004): 488-496, each of which are incorporated by reference herein in their entireties).

Embodiments as described herein can comprise a T helper epitope for generating an immune response. In embodiments, the T helper epitope comprises pan DR-binding epitope (PADRE), and/or an RGD motif RGD, for example, enhances the immunogenicity of a peptide antigen by addition of motifs that bind to cell attachment proteins. RGD provides an average of 10 times enhancement of antibody titers when incorporated into peptide antigens, such as the antigenic universal peptides of the invention. Other T helper epitopes comprise TT946-967 (FNNFTVSFWLRVPKVSASHLE; SEQ ID NO: 78) and/or TT830-844 (QYIKANSKFIGITE; SEQ ID NO: 75), which belongs to 830 to 844 or 946 to 967 amino acid sequence of the tetanus toxin Tc, human, common for most NMC molecules.

Embodiments as described herein, such as the antigenic universal peptide or composition comprising the same, can optionally comprise a protein carrier. A protein carrier can be a protein, a polypeptide, or fragment thereof that is coupled or conjugated to an antigenic universal peptide as described herein. In some embodiments, the protein carrier can be included with the vaccine composition of the invention. The protein carrier can be used to enhance the immunogenicity of the antigenic universal peptide or vaccine composition to a greater degree than the peptide or composition alone. For example, the protein carrier can serve as a T-dependent antigen which can activate and recruit T-cells and thereby augment T-cell dependent antibody production.

Non-limiting examples of protein carriers comprise bacterial toxoids, toxins, exotoxins, and nontoxic derivatives thereof, such as tetanus toxoid, tetanus toxin Fragment C, diphtheria toxoid, CRM (a nontoxic diphtheria toxin mutant) such as CRM 197, cholera toxoid, Staphylococcus aureus exotoxins or toxoids, Escherichia coli heat labile enterotoxin, Pseudomonas aeruginosa exotoxin A, including recombinantly produced, genetically detoxified variants thereof; bacterial outer membrane proteins, such as Neisseria meningitis serotype B outer membrane protein complex (OMPC), outer membrane class 3 porin (rPorB) and other porins; keyhole limpet hemocyanine (KLH), hepatitis B virus core protein, thyroglobulin, albumins, such as bovine serum albumin (BSA), human serum albumin (HSA), and ovalbumin; pneumococcal surface protein A (PspA), pneumococcal adhesin protein (PsaA); purified protein derivative of tuberculin (PPD); transferrin binding proteins, polyamino acids, such as poly(lysine:glutamic acid); peptidyl agonists of TLR-5 (e.g. flagellin of motile bacteria like Listeria); and derivatives and/or combinations of the above carriers. Preferred carriers for use in humans include tetanus toxoid, CRM 197, and OMPC. For a review of characteristics, development, and clinical trials of protein carrier of conjugate vaccines, please refer to Pichichero, Michael E. “Protein carriers of conjugate vaccines: characteristics, development, and clinical trials.” Human vaccines & immunotherapeutics 9.12 (2013): 2505-2523, which is incorporated by reference in its entirety herein.

Vaccine compositions as described herein can further comprise therapeutic and/or prophylactic agents, such as one or more antimicrobial agents. For example, the antimicrobial agent can comprise an anti-fungal agent, an anti-viral agent, an anti-bacterial agent, and/or an anti-protozoal agent.

Antifungal compounds can be organized into several groups: polyene antifungals, azoles, allylamines, echinocandins, and other antifungal compounds. Examples of polyene antifungals (compounds with multiple conjugated double bonds) include amphotericin B, candicidin, nystatin, natamycin, and rimocidin. Examples of commonly used azoles (compounds with five-membered organic rings) include fluconazole, itraconazole, ketoconazole, miconazole, and clotrimazole. Examples of allyamines (compounds that inhibit ergosterol synthesis by inhibiting squalene synthesis) include naftifine, terbinafine and amorolfine. Echinocandins (compounds that inhibit the synthesis of glucan in the cell wall) include anidulafungin, caspofungin, and micafungin. Other commonly used antifungal compounds include griseofulvin and 5-fluorocytosine. Non-limiting examples of anti-viral agents comprise TFT, Acyclovir, gancyclovir, penciclovir, cidofovir; ribavirin, interferon, phosphonoacetate, Foscarnet, amantadine, Rimatidine, oseltamivir, Valacyclovir, Valgancyclovir, Peramivir, Zanamivir, or a combination thereof.

Non-limiting examples of anti-bacterial agents comprise aminoglycosides, fluoroquinolones, beta-lactams, macrolide, and tetracyclines.

Non-limiting examples of anti-protozoal agents comprise chloroquine, pyrimethamine, mefloquine, hydroxychloroquine, metronidazole, atovaquone, or a combination thereof.

### Antibodies

The protective role of antibodies against fungal infections such as Candida has been controversial, but the evidence is mounting in favor for this mode of protection. As a prevention strategy, protection against disease may be actively or passively acquired by vaccination and transfer of preformed antibodies (e.g., monoclonal antibodies), respectively. As a therapeutic measure, experimental evidence indicates that preformed antibodies can enhance the effectiveness of antifungal agents.

Embodiments as described herein comprise an isolated antibody or binding fragment thereof that specifically binds to an antigenic universal peptide, for example that with an amino acid sequence that is at least 80% identical in two or more different microorganisms. Examples of such functional antibody fragments include, but are not limited to, Fᵥ, single chain Fᵥ (scFv), complementarity determining regions (CDRs), V_{L} (light chain variable region), V_{H} (heavy chain variable region), Fab, F(ab′)₂, minibody, diabody, and any functional portion of an immunoglobulin peptide capable of binding to target antigen. Non-limiting examples of such peptides are described herein and can comprise those of fructose-bisphosphate aldolase (Fba; GenBank Accession No. AOW28947; Protein Accession No. XP_722690), methyltetrahydropteroyltriglutamate homocysteine methyltransferase (Met6; GenBank Access No. AOW30921; Protein Accession No. XP_718219), hyphal wall protein-1 (Hwp1; GenBank Access No. ACN63125), enolase (Enol; GenBank Accession No. AAA71939), glyceraldehyde-3-phosphate dehydrogenase (Gap1; GenBank Accession No. AOW29704), and phosphoglycerate kinase (Pgk1; GenBank Accession No. AAA66523), such as the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 79, SEQ ID NO: 26, SEQ ID NO: 27, SEQ ID NO: 28, SEQ ID NO: 81, or SEQ ID NO: 82.

An antibody is a large, Y-shaped glycoprotein produced mainly by plasma cells that is used by the immune system to neutralize pathogens. It belongs to the immunoglobulin superfamily and consists of two identical heavy chains and two identical light chains, and each chain further comprising a variable region (Fᵥ) and a constant region (Fc). Five major antibody classes have been identified and include: IgA, IgD, IgE, IgG and IgM. This classification is based on differences in amino acid sequence in the constant region (Fc) of the antibody heavy chains. Based on differences in the amino acid sequence in the light chain Fc, immunoglobulins can be further classified by the type of light chain (e.g., kappa light chain (LC_{κ}) or lambda light chain (LC_{λ})). The ratio of these two light chains differs greatly among species, but the light chains are always either both kappa or both lambda.

The term “antibody” can refer to polyclonal and monoclonal antibodies and derivatives thereof (for example chimeric, humanized, and fully human antibodies). An antibody, for example, can include an immunoglobulin molecule (such as IgG (as well as subtypes IgG1, IgG2a, and IgG2b, IgG3, IgG4), IgM, IgA (as well as subtypes IgA1 and IgGA2), IgD, and IgE or any functional fragment of an immunoglobulin molecule which binds to the target antigen and or combinations thereof. Examples of such functional antibody fragments include, but are not limited to, Fᵥ, single chain Fᵥ (scFv), complementarity determining regions (CDRs), V_{L} (light chain variable region), V_{H} (heavy chain variable region), Fab, F(ab′)₂, minibody, diabody, and any functional portion of an immunoglobulin peptide capable of binding to target antigen.

Antibodies exist, e.g., as intact immunoglobulins or as a number of well characterized antibody fragments produced synthetically or by digestion with various peptidases. Thus, for example, pepsin digests an antibody below the disulfide linkages in the hinge region to produce F(ab′)₂a dimer of Fab, which itself is a light chain joined to V_{H}-CH₁ by a disulfide bond. The F(ab′)₂ may be reduced under mild conditions to break the disulfide linkage in the hinge region, thereby converting the F(ab′)₂ dimer into an Fab¹ monomer. The Fab¹ monomer is essentially an Fab with part of the hinge region (see, FUNDAMENTAL IMMUNOLOGY, 3RD ED., W. E. Paul, ed, Raven Press, N.Y. (1993), which is incorporated herein in its entirety). While various antibody fragments are defined in terms of the digestion of an intact antibody, one of skill in the art will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An antibody can be “reactive with” or “bind to” an antigen if it interacts with the antigen. This interaction is analogous to a chemical reaction in which two reactants come together to form a product. In the case of the antibody-antigen interaction, the product of the interaction is an antibody-antigen complex. Such antigen-antibody interactions can be measured by surface plasmon resonance, binding affinity assays, ELISA, Western blot, Immunofluorescence, or fluorescence-activated cell sorting (FACS) analysis.

The term “humanized” refers to an antibody wherein the constant regions have at least about 80% or greater homology to a human immunoglobulin. Additionally, some of the nonhuman, such as murine, variable region amino acid residues can be modified to contain amino acid residues of human origin.

Humanized antibodies can be referred to as “reshaped” antibodies. Manipulation of the complementarity-determining regions (CDR) is a way of achieving humanized antibodies. See, for example, Jones, et al., Nature 321:522 (1988) and Riechmann, et al., Nature 332:323 (1988), both of which are incorporated by reference herein. For a review article concerning humanized antibodies, see Winter & Milstein, Nature 349:293 (1991), incorporated by reference herein. In some embodiments, the antibodies of the invention can be humanized according to methods well known in the art, for example by modifying the amino acids of CDR regions to amino acid residues of human origin. Humanized monoclonal antibodies can also be made using the XenoMouse transgenic animal as described in U.S. Pat. No. 5,939,598, or using methods as described in US 20140004123 or EP 2003960, each of which are hereby incorporated by reference in their entireties.

The term “fully human” refers to an antibody wherein the constant regions are homologous to a human immunoglobulin. Additionally, the variable region amino acid residues are amino acid residues of human origin. Fully human antibodies can be generated using Complementarity-determining region (CDR) engraftment (N. R. Gonzales et al., Mol. Immunol. 41, 863-872 (2004); S. V. Kashmiri et al., Methods 36, 25-34 (2005); and J. Osbourn, M. Groves, and T. Vaughan, Methods 36, 61-68 (2005)); transgenic mice with human immunoglobulin genes (D. M. Fishwild et al., Nat. Biotechnol. 14, 845-851 (1996); L. L. Green, J. Immunol. Methods 231, 11-23 (1999); L. L. Green et al., Nat. Genet. 7, 13-21 (1994); A. Schedl et al., Nucleic Acids Res. 20, 3073-3077 (1992); A. Schedl et al., Nucleic Acids Res. 21, 4783-4787 (1993); A. Schedl et al., Nature 362, 258-261 (1993); and L. M. Weiner, J. Immunother. 29, 1-9 (2006)); and/or phage, yeast, or ribosome display technologies (J. Osbourn, M. Groves, and T. Vaughan, Methods 36, 61-68 (2005)).

A chimeric antibody is an antibody made by fusing the antigen binding region (for example, the variable domains of the heavy and light chains, VH and VL) from one species like a mouse, with the constant domain (effector region) from another species such as a human. In some embodiments, a chimeric antibody comprises human (or humanized) VH and VL regions, and the Fc region of a mouse. The chimeric antibodies retain the original antibody's antigen specificity and affinity.

The terms “isolated” or “substantially purified,” for example when applied to a protein such as an antibody, denotes that the protein is essentially free of other cellular components with which it is associated in the natural state. It is preferably in a homogeneous state, although it can be in either a dry or aqueous solution. Purity and homogeneity can be determined using analytical chemistry techniques such as polyacrylamide gel electrophoresis or high performance liquid chromatography. A protein which is the predominant species present in a preparation is substantially purified.

“Monoclonal antibody” can refer to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567, each of which are incorporated herein by reference in their entireties. The monoclonal antibodies may also be isolated from phage libraries generated using the techniques described in McCafferty et al., 1990, Nature 348:552-554, for example. Monoclonal antibodies, for example fully human antibodies, can also be made using transgenic mice as described in Bruggemann et al., (Arch Immunol Ther Exp (Warsz). 2015; 63(2): 101-108), or using methods as described in EP 2003960 or U.S. Pat. No. 9,220,244, each of which are hereby incorporated by reference in their entireties. Monoclonal antibodies, for example humanized monoclonal antibodies, can also be made using the XenoMouse transgenic animal as described in U.S. Pat. No. 5,939,598, or using the methods as described in U.S. Pat. No. 6,150,584 or U.S. Pat. No. 6,075,181, each of which are hereby incorporated by reference in their entireties.

A “variable region” of an antibody can refer to the variable region of the antibody light chain or the variable region of the antibody heavy chain, either alone or in combination. The variable regions of the heavy and light chain each consist of four framework regions (FR) connected by three complementary determining regions (CDRs) that contain hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al, 1997, J. Molec. Biol. 273:927-948). As used herein, a CDR may refer to CDRs defined by either approach or by a combination of both approaches.

The amino acid sequence of the monoclonal antibodies are provided herein (see Tables 20-27); the amino acid sequences of the heavy and light chain complementary determining regions CDRs of the CTLA-4 antibodies are underlined (CDR1), underlined and bolded (CDR2), or underlined, italicized, and bolded (CDR3) below:

TABLE 20
2B10C1 Ab Variable Region amino acid sequences
V_{H} chain of 2B10C1
QVQLQQPGAEVVRPGASVKVSCKASGYTVSSYWMSWVKORPEQGLEW
IGRIDPYDSETHYNQKFKDKAILTVDKSSSTAYMQLSSLTSEDSAVY
YCARTAASFDYWGQGTTLTVSS (SEQ ID NO: 257)
V_{L} chain of 2B10C1
DVVMTQTPLSLPVSLGDQASISCRSSQSLVHSNGNSYLHWYLQKPGQ
SPKLLIYKVSNRFSGVPDRFSGSGSGTDFTLNISRVEAEDLGVYFCS
QSTHVPFTFGSGTKLEIK (SEQ ID NO: 259)

TABLE 21
1D4H5 Ab Variable Region amino acid sequences
V_{H} chain of 1D4H5
EVKLEESGGGLVQPGGSMKLSCVASGFTFSNYWMNWVRQSPEKGLEW
VAEIRLKSNNYATHYAESVKGRFTISSDDSKSSVYLQMNNLRAEDTG
IYYCSTGNYWGQGTTLTVSS (SEQ ID NO: 261)
V_{L} chain of 1D4H5
EIVLTQSPTTMAASPGEKITITCSASSTISSNYLHWYQQKPGFSPKL
LIYRTSNLASGVPARFSGSGSGTSYSLTIGTMEAEDVATYYCQQGST
ISRTFGSGTKLEIK (SEQ ID NO: 263)

TABLE 22
2D5F7 Ab Variable Region amino acid sequences
V_{H} chain of 2D5F7
QVQLQQSAAELARPGASVKMSCKASGYTFSSYTMHWVKQRPGQGLEW
IGYINPSSGYTDYNQKFKDKTTLTADKSSSTAYMQLSSLTSEDSAVY
YCARLYDNYDYYAMDYWGQGTSVTVSS (SEQ ID NO: 265)
V_{L} chain of 2D5F7
DIVMSQSPSSLAVSAGEKVTMSCKSSQSLLNSRIRKNYLAWYQQKPG
QSPKLLIYWASTRESGVPDRFTGSGSGTDFTLTISSVQADDLAVYYC
KQSYNLLTFGAGTKLELK (SEQ ID NO: 267)

TABLE 23
10E7E2 Variable Region amino acid sequences
V_{H} chain of 10E7E2
QVQLKQSGPGLVQPSQSLSITCTVSGFSLTSYGVHWVRQSPGKGLEW
LGVIWSGGTTDYNAAFISRLSISKDNSKSQVFFKMNSLQANDTAIYY
CARGGHRGFAYWGQGTLVTVSA (SEQ ID NO: 269)
V_{L} chain of 10E7E2
DIVMTQSHKFMSTSVGDRVSITCKASQDVGTAVAWYQQKPGQSPKLL
IYWASTRHTGVPDRFTGSGSGTDFTLTISNVQSEDLADYFCQQYSSY
PLTFGAGTKLELK (SEQ ID NO: 271)

TABLE 24
7C6E8 Ab Variable Region amino acid sequences
V_{H} chain of 7C6E8
QIQLVQSGPELKKPGETVKISCKASGYTFTDYSMHWVKQTPGKGLKW
MGWINTETGEPTYADDFKGRFAFSLETSASTAYLQINNLKNEDTATY
FCARNYYDTTGFAYWGQGTLVTVSA (SEQ ID NO: 108)
V_{L} 1 chain of 7C6E8
DVVMTQTPLTLSVTVGQPASISCKSSQRLLYSYGKTYLNWLLQRPGQ
SPKRLIYLVSKLDSGVPDRFTGSGSGTDFTLKISRVEAEDLGVYYCV
QDTHFPYTFGGGTKLEIK (SEQ ID NO: 109)
V_{L} 2 chain of 7C6E8
DILLTQSPAILSVSPGERVSFSCRASQSIGTSIHWYQQRTNGSPRLL
IKFASESISGIPSRFSGSGSGTDFPLSINSVESEDIAGYYCQQSNSW
PTYTFGGGTKLEIK (SEQ ID NO: 110)

TABLE 25
9F2G5 Ab Variable Region amino acid sequences
V_{H} chain of 9F2G5
EVHLQQSGPELVKPGASVKISCKTSGYIFTEYTMHWVKQSHGKSLEW
IGGINPNNGGTRYNQKFKGKATLTVDKSSSTAYMELRSLTSEDSAVY
YCAWYFPYWGQGTLVTVSA (SEQ ID NO: 111)
V_{L} chain of 9F2G5
DVVMTQTPLTLSVTLGQPASISCRSSQSLLDSDGKTYLNWLLQRPGQ
SPKRLIYLVSKLDSGVPDRFTGSGSGTDFTLKISRVEAEDLGVYYCW
QGTHFPWTFGGGTKLEIK (SEQ ID NO: 112)

TABLE 26
6H1G8 Ab Variable Region amino acid sequences
V_{H} chain of 6H1G8
EVQLQQSGPELVKPGASVKISCKASGYSITGYYMHWVKQSHVKSLEW
IGRINPYTGATSYTQNFKDKASLTVDKSSSTAYMELLSLKSEDSAVY
YCAKGGGRSSYWYFDVWGAGTAVTVSS (SEQ ID NO: 113)
V_{L} chain of 6H1G8
DIQMTQTTSSLSASLGDRVTISCSASQAISNYLNWYQQKPDGTIKLL
IYYTSSLHSGVPSRFSGSGSGTDYSLTISNLEPEDIATYYCQQYSKL
PWTFGGGTKLEIK (SEQ ID NO: 114)

TABLE 27
7H6A2 Ab Variable Region amino acid sequences
V_{H} chain of 7H6A2
QIQLVQSGPELKKPGETVKISCKASGYTFTDFSMHWVKQAPGKGLKW
MGWINTETVEPTYADDFKGRFAFSLETSASTAYLQINNLKNEDTATY
FCSRVSYDGYYGDFAMDYWSQGTSVTVSS (SEQ ID NO: 115)
V_{L} chain of 7H6A2
DIQMTQTTSSLSASLGDRVTISCRASQDISNYLNWYQQKPDGTVKLL
IYYTSRLHSGVPSRFSGSGSGTDYSLTISNLEQEDIATYFCQQGNTL
PWTFGGGTKLEIK (SEQ ID NO: 116)

The nucleic acid sequence of the monoclonal CTLA-4 antibodies are provided herein (see Tables 28-35):

TABLE 28
2B10C1 Ab Variable Region nucleic acid sequences
V_{H} chain of 2B10C1
CAGGTCCAACTGCAGCAGCCTGGGGCTGAGGTGGTGAGGCCTGGGGC
TTCAGTGAAGGTGTCCTGCAAGGCTTCTGGCTACACGGTCAGCAGCT
ACTGGATGAGCTGGGTTAAGCAGAGGCCGGAGCAAGGCCTTGAGTGG
ATTGGAAGGATTGATCCTTACGATAGTGAAACTCACTACAATCAAAA
GTTCAAGGACAAGGCCATATTGACTGTAGACAAATCCTCCAGCACAG
CCTACATGCAACTCAGCAGCCTGACATCTGAGGACTCTGCGGTCTAT
TACTGTGCAAGGACGGCCGCTTCGTTTGACTATTGGGGCCAAGGCAC
CACTCTCACAGTCTCCTCA (SEQ ID NO: 256)
V_{L} chain of 2B10C1
GATGTTGTGATGACCCAAACTCCACTCTCCCTGCCTGTCAGTCTTGG
AGATCAAGCCTCCATCTCTTGCAGATCTAGTCAGAGCCTTGTACACA
GTAATGGAAACTCCTATTTACATTGGTACCTGCAGAAGCCAGGCCAG
TCTCCAAAGCTCCTGATCTACAAAGTTTCCAACCGATTTTCTGGGGT
CCCAGACAGGTTCAGTGGCAGTGGATCAGGGACAGATTTCACACTCA
ATATCAGCAGAGTGGAGGCTGAGGATCTGGGAGTTTATTTCTGCTCT
CAAAGTACACATGTTCCATTCACGTTCGGCTCGGGGACAAAGTTGGA
AATAAAA (SEQ ID NO: 258)

TABLE 29
1D4H5 Ab Variable Region nucleic acid sequences
V_{H} chain of 1D4H5
GAAGTGAAGCTTGAGGAGTCTGGAGGAGGCTTGGTGCAACCTGGAGGATC
CATGAAACTCTCCTGTGTTGCCTCTGGATTCACTTTCAGTAACTACTGGA
TGAACTGGGTCCGCCAGTCTCCAGAGAAGGGGCTTGAGTGGGTTGCTGAA
ATTAGATTGAAATCTAATAATTATGCAACACATTATGCGGAGTCTGTGAA
AGGGAGGTTCACCATCTCAAGCGATGATTCCAAAAGTAGTGTCTACCTGC
AAATGAACAACTTAAGAGCTGAAGACACTGGCATTTATTACTGTTCAACT
GGGAACTACTGGGGCCAAGGCACCACTCTCACAGTCTCCTCA
(SEQ ID NO: 260)
V_{L} chain of 1D4H5
GAAATTGTGCTCACCCAGTCTCCAACCACCATGGCTGCATCTCCCGGGGA
GAAGATCACTATCACCTGCAGTGCCAGCTCAACTATAAGTTCCAATTACT
TGCATTGGTATCAGCAGAAGCCAGGATTCTCCCCTAAACTCTTGATTTAT
AGGACATCCAATCTGGCTTCTGGAGTCCCAGCTCGCTTCAGTGGCAGTGG
GTCTGGGACCTCTTACTCTCTCACAATTGGCACCATGGAGGCTGAAGATG
TTGCCACTTACTACTGCCAGCAGGGTAGTACTATATCACGCACGTTCGGC
TCGGGGACAAAGTTGGAAATAAAA (SEQ ID NO: 262)

TABLE 30
2D5F7 Ab Variable Region nucleic acid sequences
V_{H} chain of 2D5F7
CAGGTCCAGCTGCAGCAGTCTGCAGCTGAACTGGCAAGACCTGGGGCCTC
AGTGAAGATGTCCTGCAAGGCTTCTGGCTACACCTTTAGTAGCTACACGA
TGCACTGGGTAAAACAGAGGCCTGGACAGGGTCTGGAATGGATTGGATAC
ATTAATCCTAGCAGTGGATATACTGATTACAATCAGAAGTTCAAGGACAA
GACCACATTGACTGCAGACAAATCCTCCAGCACAGCCTACATGCAACTGA
GCAGCCTGACATCTGAGGACTCTGCGGTCTATTACTGTGCAAGACTATAT
GATAACTACGATTACTATGCTATGGACTACTGGGGTCAAGGAACCTCAGT
CACCGTCTCCTCA (SEQ ID NO: 264)
V_{L} chain of 2D5F7
GACATTGTGATGTCACAGTCTCCATCCTCCCTGGCTGTGTCAGCAGGAGA
GAAGGTCACTATGAGCTGCAAATCCAGTCAGAGTCTGCTCAATAGTAGAA
TCCGAAAGAACTACTTGGCTTGGTACCAGCAGAAACCAGGGCAGTCTCCT
AAACTGCTGATCTACTGGGCATCCACTAGGGAATCTGGGGTCCCTGATCG
CTTCACAGGCAGTGGATCTGGGACAGATTTCACTCTCACCATCAGCAGTG
TGCAGGCTGATGACCTGGCAGTTTATTACTGCAAGCAATCTTATAATCTG
CTCACGTTCGGTGCTGGGACCAAGCTGGAGCTGAAA (SEQ ID NO:
266)

TABLE 31
10E7E2 Ab Variable Region nucleic acid sequences
V_{H} chain of 10E7E2
CAGGTGCAGCTGAAGCAGTCAGGACCTGGCCTAGTGCAGCCCTCACAGAG
CCTGTCCATCACCTGCACAGTCTCTGGTTTCTCATTAACTAGCTATGGTG
TACACTGGGTTCGCCAGTCTCCAGGAAAGGGTCTGGAGTGGCTGGGAGTG
ATATGGAGTGGTGGAACTACAGACTATAATGCAGCTTTCATATCCAGACT
GAGCATCAGCAAGGACAATTCCAAGAGCCAAGTTTTCTTTAAAATGAACA
GTCTGCAAGCTAATGACACAGCCATATATTACTGTGCCAGAGGGGGGCAC
CGAGGGTTTGCTTACTGGGGCCAAGGGACTCTGGTCACTGTCTCTGCA
(SEQ ID NO: 268)
V_{L} chain of 10E7E2
GACATTGTGATGACCCAGTCTCACAAATTCATGTCCACATCAGTAGGAGA
CAGGGTCAGCATCACCTGCAAGGCCAGTCAAGATGTGGGTACTGCTGTAG
CCTGGTATCAACAGAAACCAGGGCAATCTCCTAAACTACTGATTTACTGG
GCATCCACCCGGCACACTGGAGTCCCTGATCGCTTCACAGGCAGTGGATC
TGGGACAGATTTCACTCTCACCATTAGCAATGTGCAGTCTGAAGACTTGG
CAGATTATTTCTGTCAGCAATATAGCAGCTATCCTCTCACGTTCGGTGCT
GGGACCAAGCTGGAGCTGAAA (SEQ ID NO: 270)

TABLE 32
7C6E8 Ab Variable Region nucleic acid sequences
V_{H} chain of 7C6H8
CAGATCCAGTTGGTGCAGTCTGGACCTGAGCTGAAGAAGCCTGGAGAGAC
AGTCAAGATCTCCTGCAAGGCTTCTGGTTATACCTTCACAGACTATTCAA
TGCACTGGGTGAAGCAGACTCCAGGAAAGGGTTTAAAGTGGATGGGCTGG
ATAAATACTGAGACTGGTGAGCCAACATATGCAGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCTAGAAACTAC
TATGATACGACCGGGTTTGCTTACTGGGGCCAAGGGACTCTGGTCACTGT
CTCTGCA (SEQ ID NO: 125)
V_{L} 1 chain of 7C6E8
GATGTTGTGATGACCCAGACTCCACTCACTTTGTCGGTTACCGTTGGACA
ACCAGCCTCTATCTCTTGCAAGTCAAGTCAGCGCCTCTTATATAGTTATG
GAAAAACCTATTTGAATTGGTTATTACAGAGGCCAGGCCAGTCTCCAAAG
CGCCTAATCTATCTGGTGTCTAAACTGGACTCTGGAGTCCCTGACAGGTT
CACTGGCAGTGGATCAGGAACAGATTTTACACTGAAAATCAGCAGAGTGG
AGGCTGAGGATTTGGGAGTTTATTACTGCGTGCAAGATACACATTTTCCG
TACACGTTCGGAGGGGGGACCAAGCTGGAAATAAAA (SEQ ID NO:
126)
V_{L} 2 chain of 7C6E8
GACATCTTGCTGACTCAGTCTCCAGCCATCCTGTCTGTGAGTCCAGGAGA
AAGAGTCAGTTTCTCCTGCAGGGCCAGTCAGAGCATTGGCACAAGCATAC
ACTGGTATCAGCAAAGAACGAATGGTTCTCCAAGGCTTCTCATAAAGTTT
GCTTCTGAGTCTATCTCTGGGATCCCTTCCAGGTTTAGTGGCAGTGGATC
AGGGACAGATTTTCCTCTTAGCATCAACAGTGTGGAGTCTGAAGATATTG
CAGGTTATTACTGTCAACAAAGTAATAGCTGGCCAACGTACACGTTCGGA
GGGGGGACCAAGCTGGAAATAAAA (SEQ ID NO: 127)

TABLE 33
9F2G5 Ab Variable Region nucleic acid sequences
V_{H} chain of 9F2G5
GAGGTCCACCTGCAACAGTCTGGACCTGAGCTGGTGAAGCCTGGGGCTTC
AGTGAAGATATCCTGCAAGACTTCTGGATACATATTCACTGAATACACCA
TGCACTGGGTGAAGCAGAGCCATGGAAAGAGCCTTGAGTGGATTGGAGGT
ATTAATCCTAACAATGGTGGTACTAGGTACAACCAGAAGTTCAAGGGCAA
GGCCACATTGACTGTAGACAAGTCCTCCAGCACAGCCTACATGGAGCTCC
GCAGCCTGACATCTGAGGATTCTGCAGTCTATTACTGTGCTTGGTATTTT
CCTTACTGGGGCCAAGGGACTCTGGTCACTGTCTCTGCA (SEQ ID
NO: 128)
V_{L} chain of 9F2G5
GATGTTGTGATGACCCAGACTCCACTCACTTTGTCGGTTACCCTTGGACA
ACCAGCCTCCATCTCTTGCAGGTCAAGTCAGAGCCTCTTAGATAGTGATG
GAAAGACATATTTGAATTGGTTGTTACAGAGGCCAGGCCAGTCTCCAAAG
CGCCTAATCTATCTGGTGTCTAAACTGGACTCTGGAGTCCCTGACAGGTT
CACTGGCAGTGGATCAGGGACAGATTTCACACTGAAAATCAGCAGAGTGG
AGGCTGAGGATTTGGGAGTTTATTATTGCTGGCAAGGTACACATTTTCCG
TGGACGTTCGGTGGAGGCACCAAGCTGGAAATCAAA (SEQ ID NO:
129)

TABLE 34
6H1G8 Ab Variable Region nucleic acid sequences
V_{H} chain of 6H1G8
GAGGTCCAGCTGCAACAGTCTGGACCTGAGCTGGTGAAGCCTGGGGCCTC
AGTGAAGATATCCTGCAAGGCTTCTGGTTACTCAATCACTGGCTACTACA
TGCACTGGGTGAAGCAAAGCCATGTAAAGAGCCTTGAGTGGATTGGACGT
ATTAATCCTTACACTGGTGCTACTAGCTACACCCAGAATTTCAAGGACAA
GGCCAGCTTGACTGTAGATAAGTCCTCCAGCACAGCCTACATGGAGCTCC
TCAGCCTGAAATCTGAAGACTCTGCAGTCTATTACTGTGCGAAGGGGGGC
GGTAGGAGCTCCTACTGGTACTTCGATGTCTGGGGCGCAGGGACCGCGGT
CACCGTCTCCTCA (SEQ ID NO: 130)
V_{L} chain of 6H1G8
GATATCCAGATGACACAGACTACATCCTCCCTGTCTGCCTCTCTGGGAGA
CAGAGTCACCATCAGTTGCAGTGCAAGTCAGGCCATTAGCAATTATTTAA
ACTGGTATCAGCAGAAACCAGATGGAACTATTAAACTCCTGATCTATTAC
ACATCAAGTTTACACTCAGGAGTCCCATCAAGGTTCAGTGGCAGTGGGTC
TGGGACAGATTATTCTCTCACCATCAGCAACCTGGAACCTGAAGATATTG
CCACTTACTATTGTCAGCAGTATAGTAAACTTCCGTGGACGTTCGGTGGA
GGCACCAAGCTGGAAATCAAA (SEQ ID NO: 131)

TABLE 35
7H6A2 Ab Variable Region nucleic acid sequences
V_{H} chain of 7H6A2
CAGATCCAGTTGGTGCAGTCTGGACCTGAGCTGAAGAAGCCTGGAGAGAC
AGTCAAGATCTCCTGCAAGGCTTCTGGTTATACCTTCACAGACTTTTCAA
TGCACTGGGTGAAGCAGGCTCCAGGAAAGGGTTTAAAGTGGATGGGCTGG
ATAAACACTGAGACTGTTGAGCCAACATATGCAGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTTCTAGAGTCTCC
TATGATGGTTACTATGGGGACTTTGCTATGGACTACTGGAGTCAAGGAAC
CTCAGTCACCGTCTCCTCA (SEQ ID NO: 132)
V_{L} chain of 7H6A2
GATATCCAGATGACACAGACTACATCCTCCCTGTCTGCCTCTCTGGGAGA
CAGAGTCACCATCAGTTGCAGGGCAAGTCAGGACATTAGCAATTATTTAA
ACTGGTATCAGCAGAAACCAGATGGAACTGTTAAACTCCTGATCTACTAC
ACATCAAGATTACACTCAGGAGTCCCATCAAGGTTCAGTGGCAGTGGGTC
TGGAACAGATTATTCTCTCACCATTAGCAACCTGGAGCAAGAAGATATTG
CCACTTACTTTTGCCAACAGGGTAATACGCTTCCTTGGACGTTCGGTGGA
GGCACCAAGCTGGAAATCAAA (SEQ ID NO: 133)

The amino acid sequences of the heavy and light chain complementary determining regions of the monoclonal antibodies are shown in Table 36A-B below:

TABLE 36A
Heavy chain (V_{H}) complementary determining
regions (CDRs) of the antibodies

| Sequence | | | |
|---|---|---|---|
| ID | V_{H} CDR1 | V_{H} CDR2 | V_{H} CDR3 |
| 2B10C1 | SYWMS | RIDPYDSETHYNQK | TAASEDY |
| | (SEQ ID | FKD (SEQ ID | (SEQ ID |
| | NO: 134) | NO: 135) | NO: 136) |
| 1D4H5 | NYWMN | EIRLKSNNYATHYA | GNY |
| | (SEQ ID | ESVKG (SEQ ID | |
| | NO: 137) | NO: 138) | |
| 2D5F7 | SYTMH | YINPSSGYTDYNQK | LYDNYDYY |
| | (SEQ ID | FKD (SEQ ID | AMDY |
| | NO: 140) | NO: 141) | (SEQ ID |
| | | | NO: 142) |
| 10E7E2 | SYGVH | VIWSGGTTDYNAAF | GGHRGFAY |
| | (SEQ ID | IS (SEQ ID NO: | (SEQ ID |
| | NO: 143) | 144) | NO: 145) |
| 7C6E8 | DYSMH | WINTETGEPTYADD | NYYDTTGF |
| | (SEQ ID | FKG (SEQ ID | AY (SEQ |
| | NO: 146) | NO: 147) | ID NO: |
| | | | 148) |
| 9F2G5 | EYTMH | GINPNNGGTRYNQK | YFPY |
| | (SEQ ID | FKG (SEQ ID | (SEQ ID |
| | NO: 149) | NO: 150) | NO: 151) |
| 6H1G8 | GYYMH | RINPYTGATSYTQN | GGGRSSYW |
| | (SEQ ID | FKD (SEQ ID | YFDV |
| | NO: 152) | NO: 153) | (SEQ ID |
| | | | NO: 154) |
| 7H6A2 | DFSMH | WINTETVEPTYADD | VSYDGYYG |
| | (SEQ ID | FKG (SEQ ID | DFAMDY |
| | NO: 155) | NO: 156) | (SEQ ID |
| | | | NO: 157) |

TABLE 36B
Light chain (V_{L}) complementary determining
regions (CDRs) of the antibodies

| Sequence | | | |
|---|---|---|---|
| ID | V_{L} CDR1 | V_{L} CDR2 | V_{L} CDR3 |
| 2B10C1 | RSSQSLVHSNG | KVSNRFS | SQSTHVPFT |
| | NSYLH (SEQ | (SEQ ID | (SEQ ID |
| | ID NO: 158) | NO: 159) | NO: 160) |
| 1D4H5 | SASSTISSNYL | RTSNLAS | QQGSTISRT |
| | H (SEQ ID | (SE QID | (SEQ ID |
| | NO: 161) | NO: 162) | NO: 163) |
| 2D5F7 | KSSQSLLNSRI | WASTRES | KQSYNLLT |
| | RKNYLA (SEQ | (SEQ ID | (SEQ ID |
| | ID NO: 164) | NO: 165) | NO: 166) |
| 10E7E2 | KASQDVGTAVA | WASTRHT | QQYSSYPLT |
| | (SEQ ID NO: | (SEQ ID | (SEQ ID |
| | 167) | NO: 168) | NO: 169) |
| 7C6E8-1 | KSSQRLLYSYG | LVSKLDS | VQDTHEPYT |
| | KTYLN (SEQ | (SEQ ID | (SEQ ID |
| | ID NO: 170) | NO: 171) | NO: 172) |
| 7C6E8-2 | RASQSIGTSIH | FASESIS | QQSNSWPTY |
| | (SEQ ID NO: | (SEQ ID | T (SEQ ID |
| | 173) | NO: 174) | NO: 175) |
| 9F2G5 | RSSQSLLDSDG | LVSKLDS | WQGTHEPWT |
| | KTYLN (SEQ | (SEQ ID | (SEQ ID |
| | ID NO: 176) | NO: 177) | NO: 178) |
| 6H1G8 | SASQAISNYLN | YTSSLHS | QQYSKLPWT |
| | (SEQ ID NO: | (SEQ ID | (SEQ ID |
| | 179) | NO: 180) | NO: 181) |
| 7H6A2 | RASQDISNYLN | YTSRLHS | QQGNTLPWT |
| | (SEQ ID NO: | (SEQ ID | (SEQ ID |
| | 182) | NO: 183) | NO: 184) |

The nucleic acid sequences of the heavy and light chain complementary determining regions of the monoclonal antibodies are shown in Table 37A-B below:

TABLE 37A
Heavy chain (V_{H}) complementary determining
regions (CDRs) of the antibodies

| Sequence | | | |
|---|---|---|---|
| ID | V_{H} CDR1 | V_{H} CDR2 | V_{H} CDR3 |
| 2B10C1 | AGCTACTG | AGGATTGATCCTTA | ACGGCCGCTTCGTT |
| | GATGAGC | CGATAGTGAAACTC | TGACTAT (SEQ |
| | (SEQ ID | ACTACAATCAAAAG | ID NO: 187) |
| | NO: 185) | TTCAAGGAC (SEQ | |
| | | ID NO: 186) | |
| 1D4H5 | AACTACTG | GAAATTAGATTGAA | GGGAACTAC |
| | GATGAAC | ATCTAATAATTATG | |
| | (SEQ ID | CAACACATTATGCG | |
| | NO: 188) | GAGTCTGTGAAAGG | |
| | | G (SEQ ID NO: | |
| | | 189) | |
| 2D5F7 | AGCTACAC | TACATTAATCCTAG | CTATATGATAACTA |
| | GATGCAC | CAGTGGATATACTG | CGATTACTATGCTA |
| | (SEQ ID | ATTACAATCAGAAG | TGGACTAC (SEQ |
| | NO: 191) | TTCAAGGAC (SEQ | ID NO: 193) |
| | | ID NO: 192) | |
| 10E7E2 | AGCTATGG | GTGATATGGAGTGG | GGGGGGCACCGAGG |
| | TGTACAC | TGGAACTACAGACT | GTTTGCTTAC |
| | (SEQ ID | ATAATGCAGCTTTC | (SEQ ID NO: |
| | NO: 194) | ATATCC (SEQ ID | 196) |
| | | NO: 195) | |
| 7C6E8 | GACTATTC | TGGATAAATACTGA | AACTACTATGATAC |
| | AATGCAC | GACTGGTGAGCCAA | GACCGGGTTTGCTT |
| | (SEQ ID | CATATGCAGATGAC | AC (SEQ ID NO: |
| | NO: 197) | TTCAAGGGA (SEQ | 199) |
| | | ID NO: 198) | |
| 9F2G5 | GAATACAC | GGTATTAATCCTAA | TATTTTCCTTAC |
| | CATGCAC | CAATGGTGGTACTA | (SEQ ID NO: |
| | (SEQ ID | GGTACAACCAGAAG | 202) |
| | NO: 200) | TTCAAGGGC (SEQ | |
| | | ID NO: 201) | |
| 6H1G8 | GGCTACTA | CGTATTAATCCTTA | GGGGGCGGTAGGAG |
| | CATGCAC | CACTGGTGCTACTA | CTCCTACTGGTACT |
| | (SEQ ID | GCTACACCCAGAAT | TCGATGTC (SEQ |
| | NO: 203) | TTCAAGGAC (SEQ | ID NO: 205) |
| | | ID NO: 204) | |
| 7H6A2 | GACTTTTC | TGGATAAACACTGA | GTCTCCTATGATGG |
| | AATGCAC | GACTGTTGAGCCAA | TTACTATGGGGACT |
| | (SEQ ID | CATATGCAGATGAC | TTGCTATGGACTAC |
| | NO: 206) | TTCAAGGGA (SEQ | (SEQ ID NO: |
| | | ID NO: 207) | 208) |

TABLE 37B
Light chain (V_{L}) complementary determining
regions (CDRs) of the antibodies

| Sequence | | | |
|---|---|---|---|
| ID | V_{L} CDR1 | V_{L} CDR2 | V_{L} CDR3 |
| 2B10C1 | AGATCTAGTCAGAG | AAAGTTTCCAACCG | TCTCAAAGTAC |
| | CCTTGTACACAGTA | ATTTTCT (SEQ | ACATGTTCCAT |
| | ATGGAAACTCCTAT | ID NO: 210) | TCACG (SEQ |
| | TTACAT (SEQ ID | | ID NO: 211) |
| | NO: 209) | | |
| 1D4H5 | AGTGCCAGCTCAAC | AGGACATCCAATCT | CAGCAGGGTAG |
| | TATAAGTTCCAATT | GGCTTCT (SEQ | TACTATATCAC |
| | ACTTGCAT (SEQ | ID NO: 213) | GCACG (SEQ |
| | ID NO: 212) | | ID NO: 214) |
| 2D5F7 | AAATCCAGTCAGAG | TGGGCATCCACTAG | AAGCAATCTTA |
| | TCTGCTCAATAGTA | GGAATCT (SEQ | TAATCTGCTCA |
| | GAATCCGAAAGAAC | ID NO: 216) | CG( SEQ ID |
| | TACTTGGCT (SEQ | | NO: 217) |
| | ID NO: 215) | | |
| 10E7E2 | AAGGCCAGTCAAGA | TGGGCATCCACCCG | CAGCAATATAG |
| | TGTGGGTACTGCTG | GCACACT (SEQ | CAGCTATCCTC |
| | TAGCC (SEQ ID | ID NO: 219) | TCACG (SEQ |
| | NO: 218) | | ID NO: 220) |
| 7C6E8-1 | AAGTCAAGTCAGCG | CTGGTGTCTAAACT | GTGCAAGATAC |
| | CCTCTTATATAGTT | GGACTCT (SEQ | ACATTTTCCGT |
| | ATGGAAAAACCTAT | ID NO: 222) | ACACG (SEQ |
| | TTGAAT (SEQ ID | | ID NO: 223) |
| | NO: 221) | | |
| 7C6E8-2 | AGGGCCAGTCAGAG | TTTGCTTCTGAGTC | CAACAAAGTAA |
| | CATTGGCACAAGCA | TATCTCT (SEQ | TAGCTGGCCAA |
| | TACAC (SEQ ID | ID NO: 225) | CGTACACG |
| | NO: 224) | | (SEQ ID NO: |
| | | | 226) |
| 9F2G5 | AGGTCAAGTCAGAG | CTGGTGTCTAAACT | TGGCAAGGTAC |
| | CCTCTTAGATAGTG | GGACTCT (SEQ | ACATTTTCCGT |
| | ATGGAAAGACATAT | ID NO: 228) | GGACG (SEQ |
| | TTGAAT (SEQ ID | | ID NO: 229) |
| | NO: 227) | | |
| 6H1G8 | AGTGCAAGTCAGGC | TACACATCAAGTTT | CAGCAGTATAG |
| | CATTAGCAATTATT | ACACTCA (SEQ | TAAACTTCCGT |
| | TAAAC (SEQ ID | ID NO: 231) | GGACG (SEQ |
| | NO: 230) | | ID NO: 232) |
| 7H6A2 | AGGGCAAGTCAGGA | TACACATCAAGATT | CAACAGGGTAA |
| | CATTAGCAATTATT | ACACTCA (SEQ | TACGCTTCCTT |
| | TAAAC (SEQ ID | ID NO: 234) | GGACG (SEQ |
| | NO: 233) | | ID NO: 235) |

“Constant region” of an antibody can refer to the constant region of the antibody light chain or the constant region of the antibody heavy chain, either alone or in combination.

Embodiments as described herein comprise an isolated antibody or binding fragment thereof that specifically binds to an antigenic universal peptide with an amino acid sequence that is at least 80%0 identical in two or more different microorganisms. For example, such antigenic universal peptides comprise the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 79, SEQ ID NO: 26, SEQ ID NO: 27, SEQ ID NO: 28, SEQ ID NO: 81, or SEQ ID NO: 82.

Further, embodiments as described herein comprise an isolated antibody, for example, a monoclonal antibody (such as a humanized antibody, a fully human antibody, or a chimeric antibody), a polyclonal antibody, or binding fragment thereof that specifically binds to at least one synthetic cell wall peptide, for example a bacterial cell wall peptide or a fungal cell wall peptide. Such peptides, including the antigenic universal peptides described herein, can comprise an amino acid sequence that is at least 80% identical in two or more different microorganisms. In embodiments the peptide corresponds to a cell wall protein, such as fructose-bisphosphate aldolase (Fba; GenBank Accession No. AOW28947; Protein Accession No. XP_722690), methyltetrahydropteroyltriglutamate homocysteine methyltransferase (Met6; GenBank Access No. AOW30921; Protein Accession No. XP_718219), hyphal wall protein-1 (Hwp1; GenBank Access No. ACN63125), enolase (Enol; GenBank Accession No. AAA71939), glyceraldehyde-3-phosphate dehydrogenase (Gap1; GenBank Accession No. AOW29704), and phosphoglycerate kinase (Pgk1; GenBank Accession No. AAA66523), or a combination thereof.

Binding fragments, for example, can comprise an Fᵥ fragment, a Fab fragment, a F(ab′) fragment, a F(ab′)₂ fragment, a disulfide stabilized Fᵥ protein (dsFv) fragment, a scFv fragment, a minibody fragment, or a diabody fragment. These binding fragments can bind to target peptide sequences comprising, for example the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 79, SEQ ID NO: 26, SEQ ID NO: 27, SEQ ID NO: 28, SEQ ID NO: 81, or SEQ ID NO: 82, or target peptide sequences found in fructose-bisphosphate aldolase (Fba; GenBank Accession No. AOW28947; Protein Accession No. XP_722690), methyltetrahydropteroyltriglutamate homocysteine methyltransferase (Met6; GenBank Access No. AOW30921; Protein Accession No. XP_718219), hyphal wall protein-1 (Hwp1; GenBank Access No. ACN63125), enolase (Enol; GenBank Accession No. AAA71939), glyceraldehyde-3-phosphate dehydrogenase (Gap1; GenBank Accession No. AOW29704), and phosphoglycerate kinase (Pgk1; GenBank Accession No. AAA66523), corresponding to cell wall proteins of interest.

In embodiments, the antibody or binding fragment binds to its antigen, such as the peptide antigen, with an equilibrium dissociation constant (K_{D}) of less than or equal to about 10⁻⁴ nM, between 10⁻⁴ nM and 10⁻⁶ nM, or greater than 10⁻⁶ nM. The equilibrium dissociation constant can be determined, for example, by surface plasmon resonance (SPR, e.g., BIAcore), ELISA, gel-shift assays, pull-down assays, equilibrium dialysis, analytical ultracentrifugation, spectroscopic assays, and Isothermal Titration Calorimetry.

In embodiments, the antibody or binding fragment thereof has an IC₅₀ less than or equal to 0.01 μg/ml, between about 0.01 μg/ml and 30 μg/ml, or greater than 30 μg/ml (see US 20020016681 for a discussion of such IC₅₀ measurements, which is incorporated herein by reference in its entirety).

In embodiments, the antibody or binding fragment thereof comprises the product produced by the hybridoma clones 2B10C1, 1D4H5, 2D5F7, 10E7E2, 7C6E8, 9F2G5, 6H1G8, 7H6A2, 2C9G3, 5A9D1, 5A11B1, 6E3D9, 7A3C4, 1A11H8, 10B6H8, or 5G5G3.

In embodiments, the antibody or fragment thereof produced by the clones generated in response to an antigenic universal peptide (e.g, those hybridoma clones listed in Table 3) comprises a variable domain having a variable light chain (VL) amino acid or nucleotide sequence at least 90% identical to those produced by the clones generated in response to an antigenic universal peptide or chimeric peptide, or having a variable heavy chain (VH) amino acid or nucleotide sequence at least 90% identical to those produced by the clones generated in response to an antigenic universal peptide or chimeric peptide. For example, the antibody or fragment thereof comprises a variable domain having a variable light chain (VL) amino acid or nucleotide sequence at least 90%, 91%, 92%, 93%, 94%, 95%, 96%, 97%, 98%, 99%, or 100% identical to those detailed in TABLE 1 or having a variable heavy chain (VH) amino acid or nucleotide sequence at least 90% identical to those detailed in TABLE 1. In embodiments, the variable light chain (V_{L}) and/or variable heavy chain comprises fully human framework region(s).

TABLE 1

| Clone | Chain | SEQ ID NO |
|---|---|---|
| 2B10C1 | V_{H} (DNA) | 59 |
| | V_{H} (Amino Acid) | 60 |
| | V_{L} (DNA) | 61 |
| | V_{L} (Amino Acid) | 62 |
| 1D4H5 | V_{H} (DNA) | 63 |
| | V_{H} (Amino Acid) | 64 |
| | V_{L} (DNA) | 65 |
| | V_{L} (Amino Acid) | 66 |
| 2D5F7 | V_{H} (DNA) | 67 |
| | V_{H} (Amino Acid) | 68 |
| | V_{L} (DNA) | 69 |
| | V_{L} (Amino Acid) | 70 |
| 10E7E2 | V_{H} (DNA) | 71 |
| | V_{H} (Amino Acid) | 72 |
| | V_{L} (DNA) | 73 |
| | V_{L} (Amino Acid) | 74 |
| 7C6E8 | V_{H} (DNA) | 125 |
| | V_{H} (Amino Acid) | 108 |
| | V_{L} (DNA) | 126 or 127 |
| | V_{L} (Amino Acid) | 109 or 110 |
| 9F2G5 | V_{H} (DNA) | 128 |
| | V_{H} (Amino Acid) | 111 |
| | V_{L} (DNA) | 129 |
| | V_{L} (Amino Acid) | 112 |
| 6H1G8 | V_{H} (DNA) | 130 |
| | V_{H} (Amino Acid) | 113 |
| | V_{L} (DNA) | 131 |
| | V_{L} (Amino Acid) | 114 |
| 7H6A2 | V_{H} (DNA) | 132 |
| | V_{H} (Amino Acid) | 115 |
| | V_{L} (DNA) | 133 |
| | V_{L} (Amino Acid) | 116 |

Embodiments as described herein further comprise an engineered cell, such as hybridomas, that secretes an antibody or binding fragment as described herein.

Embodiments as described herein further comprise an isolated nucleic acid encoding an antibody or binding fragment as described herein. The isolated nucleic acid, for example, can be a component of an expression vector. The isolated nucleic acid and/or the expression vector can be transformed into a host cell using methods well established in the art so as to produce the antibody or binding fragment as described herein (Sambrook et al., Molecular Cloning: A Laboratory Manual, Cold SpringHarbor Laboratory, Cold Spring Harbor, 1989). Non-limiting examples of such host cells comprise mammalian cells, such as CHO and HEK 293 cells.

Compositions comprising antibodies or binding-fragments as described herein can further comprise therapeutic and/or prophylactic agents, such as one or more antimicrobial agents. For example, the antimicrobial agent can comprise an anti-fungal agent, an anti-viral agent, an anti-bacterial agent, or an anti-protozoal agent.

Non-limiting examples of an antifungal agent can comprise at least one polyene, at least one azole, at least one allylamine, echinocardins or a combination thereof.

Non-limiting examples of anti-viral agents comprise TFT, Acyclovir, gancyclovir, penciclovir, cidofovir; ribavirin, interferon, phosphonoacetate, Foscarnet, amantadine, Rimatidine, oseltamivir, Valacyclovir, Valgancyclovir, Peramivir, Zanamivir, anti-retroviral drugs or a combination thereof.

Non-limiting examples of anti-bacterial agents comprise aminoglycosides, fluoroquinolones, beta-lactams, macrolide, and tetracyclines.

Non-limiting examples of anti-protozoal agents comprise chloroquine, pyrimethamine, mefloquine, hydroxychloroquine, metronidazole, atovaquone, or a combination thereof.

### Active Immunization

The term “active immunization” can refer to the induction of an immune response in an individual, typically an animal, elicited by the administration of an immunogen, vaccine, antigen or hapten-carrier conjugate. By contrast, passive immunization refers to the conferral of immunity in an individual by the transfer of immune molecules or cells into said individual.

Embodiments as described herein comprise antigenic universal peptide vaccines and antigenic vaccine compositions for active immunization against an infection. For example, peptides, chimeric peptides, and compositions comprising the same as described herein can be administered to a subject as a method of active immunization so as to protect against and/or treat an infection, such as a microbial infection. Non-limiting examples of such infections are described herein, and comprise fungal infections, bacterial infections, viral infections, and protozoal infections.

The pharmaceutical compositions of the present invention are advantageously administered in the form of injectable compositions. A typical composition for such purpose comprises a pharmaceutically acceptable carrier. The carrier can be a sold, liquid, solution, suspension, gel, ointment, lotion, or combinations thereof as further discussed herein. For instance, the composition may contain human serum albumin in a phosphate buffer containing NaCl. Other pharmaceutically acceptable carriers include aqueous solutions, non-toxic excipients, including salts, preservatives, buffers and the like (REMINGTON'S PHARMACEUTICAL SCIENCES, 15th Ed., Easton ed., Mack Publishing Co., pp 1405-1412 and 1461-1487 (1975) and THE NATIONAL FORMULARY XIV, 14th Ed., American Pharmaceutical Association, Washington, D.C. (1975), both hereby incorporated by reference). Examples of non-aqueous solvents are propylene glycol, polyethylene glycol, vegetable oil and injectable organic esters such as ethiolate. Aqueous carriers include water, alcoholic/aqueous solutions, saline solutions, parenteral vehicles such as sodium chloride, Ringer's dextrose, etc. Intravenous vehicles include fluid and nutrient replenishers. The pH and exact concertation of the various components the pharmaceutical composition are adjusted according to routine skills in the art. Goodman and Gilman, THE PHARMACOLOGICAL BASIS FOR THERAPEUTICS (7th ed).

Typically, such vaccines are prepared as injectables, either as liquid solutions or suspensions, solid forms suitable for solution in, or suspension in, liquid prior to injection may also be prepared. The preparation also may be emulsified. The active immunogenic ingredient is often mixed with an excipient that is pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH-buffering agents, adjuvants or immunopotentiators that enhance the effectiveness of the vaccine.

The vaccines are conventionally administered intraperitoneally, intramuscularly, intradermally, subcutaneously, orally, nasally, parenterally or administered directly to the urogenital tract, preferably topically, to stimulate mucosal immunity. Additional formulations are suitable for other modes of administration and include oral formulations. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders and contain 10%-95% of active ingredient, preferably 25-70%.

The dose to be administered depends on a predetermined quantity of active material calculated to produce the desired therapeutic effect in association with the required diluent, i.e., carrier or vehicle, and a particular treatment regimen. The quantity to be administered, both according to number of treatments and amount, depends on the subject to be treated, capacity of the subject's immune system to synthesize antibodies, and degree of protection desired. The precise amounts of active ingredient required to be administered depend on the judgment of the practitioner and are peculiar to each individual. However, suitable dosage ranges are on the order of one to several hundred micrograms of active ingredient per individual subject. Suitable regimes for initial administration and booster shots also vary but are typified by an initial administration followed in one or two week intervals by one or more subsequent injections or other administration. Annual boosters may be used for continued protection.

### Passive Immunization

“Passive immunization” can refer to conferral of immunity by the administration, by any route, of exogenously produced immune molecules (e.g. antibodies) or cells (e.g. T-cells) into an animal. Passive immunization differs from “active” immunization, where immunity is obtained by introduction of an immunogen, vaccine, antigen or hapten-carrier conjugate into an individual to elicit an immune response.

Passive immunity can occur naturally, when maternal antibodies are transferred to the fetus through the placenta, and it can also be induced artificially, when high levels of antibodies specific to a pathogen or toxin are transferred to non-immune persons through compositions and/or blood products that contain antibodies, such as in immunoglobulin therapy or antiserum therapy.

Passive immunization is used when there is a high risk of infection and insufficient time for the body to develop its own immune response, or to reduce the symptoms of ongoing or immunosuppressive diseases. Passive immunization can be provided when people cannot synthesize antibodies, and when they have been exposed to a disease that they do not have immunity against.

Artificially acquired passive immunity is a short-term immunization achieved by the transfer of antibodies, which can be administered in several forms; as human or animal blood plasma or serum, as pooled human immunoglobulin for intravenous (IVIG) or intramuscular (IG) use, as high-titer human IVIG or IG from immunized donors or from donors recovering from the disease, and as monoclonal antibodies (MAb). Passive transfer is used to prevent disease or used prophylactically in the case of immunodeficiency diseases, such as hypogammaglobulinemia. It is also used in the treatment of several types of acute infection, and to treat poisoning.

Immunity derived from passive immunization lasts for a few weeks to three to four months. Passive immunity provides immediate protection, but the body does not develop memory, therefore the patient is at risk of being infected by the same pathogen later unless they acquire active immunity or vaccination.

Embodiments as described herein comprise antibodies and compositions for passive protection against an infection. For example, antibodies and fragments thereof as described herein can be administered to a subject as a method of passive immunization so as to protect against and/or treat an infection, such as a microbial infection. Non-limiting examples of such infections are described herein, and comprise fungal infections, bacterial infections, viral infections, and protozoal infections.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Introduction

Morbidity and mortality from invasive fungal infections remain unacceptably high despite availability of antifungal agents, underscoring the need for more effective strategies to prevent and/or treat fungal infections.

Invasive fungal diseases often take hold when a subject's natural defenses are weakened, such as what is commonly seen in in hospital settings. Vaccination of high-risk groups, for example subjects admitted to or employed in hospital settings, is a particularly promising strategy to prevent and/or protect against invasive fungal infections because easily identifiable risk factors are clearly defined for many such infections, including candidiasis and aspergillosis. The predominant risk factors for disseminated candidiasis are common iatrogenic and/or nosocomial conditions that disrupt protective anatomical barriers or result in a substantial increase in the colonization burden of Candida spp., such as indwelling plastic catheters, abdominal or cardiac surgery, prolonged hospital stay, stay in an intensive care unit, and receipt of broad-spectrum antibiotics. Often, development of such risk factors precedes infection, affording a wide window of opportunity to vaccinate at-risk subjects, such as those at acute risk, before the onset of infection.

Hematogenously disseminated candidiasis in humans is the third leading cause of nosocomial bloodstream infection in the US. Aspergillus is the second most common cause of nosocomial, invasive fungal infections, with an incidence of approximately 5 per 100,000 population in the United States. Embodiments as described herein can be used to prevent and/or protect against aspergillosis infection, which has an extremely high mortality rate despite the availability of antifungal therapies. For invasive candidiasis, the mortality rate is 40% to 50%, even with appropriate anti-fungal drug treatments. For invasive aspergillosis, mortality rates range from 40% to 90% in high-risk populations, and are dependent on factors such as host immune status, the site of infection, and the treatment regimen applied Novel universal peptide/multiple-peptide vaccine candidates as described herein are expressed with 100% homology in medically important fungi, such as Candida spp. and Aspergillus spp. Non-limiting examples of each comprise C. albican, C. glabrata, C. tropicalis, C. parapsilosis, C. krusei, C. dubliniensis, C. auris, C. parapsilosis, A. fumigatus, A. flavus, A. niger, A. terreus, and A. lentulus.

The synthetic peptide and multi-peptide vaccines against Candida spp. protect mice against disseminated candidiasis. Further, antibodies specific for the universal peptides [for example, GPV-MO1 (SEQ ID NO: 81; PTTTIGSFPQ); GPV-M02 (SEQ ID NO. 79; NPDCGLKTR); GPV-M1 (SEQ ID NO. 1; FWVNPDCGLKTR), GPV-M2 (SEQ ID NO. 2; TTTIGSFPQTKDIR)] are each capable of protecting the animals. Without wishing to be bound by theory, we will isolate universal peptide-related monoclonal antibodies that can offer immediate protection by administration of MAbs (i.e., passive protection, or passive immunization). Such compositions and methods provide strong alternative measures to conventional antifungal drug therapy.

With the aging global and US populations, increasingly intensive medical treatments of critical illnesses, for example increasingly aggressive immune-suppressive treatment of patients with cancer, the incidence of invasive fungal infections is expected to continue to rise over the coming decades. Mortality rates associated with these fungal infections remain high despite the availability of new antifungal agents. These factors underscore the important of the development of vaccine compositions and methods to prevent and/or protect against fungal infections.

### Peptide Vaccines Against Fungal Species

Synthetic universal peptide and double-peptide vaccines, such as fully synthetic peptide and double-peptide vaccines, against Candida spp cell surface epitopes have been successfully designed and tested to protect mice against disseminated candidiasis. In detail, a panel of peptides that are universally produced by medically important Candida species in addition to C. albicans, as well as Aspergillus spp. have been defined. Universal peptide/multiple-peptide vaccines described herein are expressed with 100% amino acid sequence homology in medically important fungi, including medically important Candida spp, Aspergillus fumigates, Aspergil illus niger and other Aspergillus spp.

A panel of fully synthetic universal peptide vaccines (for example, GPV-MO1, GPV-M02, GPV-M1, GPV-M2, GPV-P11, GPV-P2) and a double-peptide vaccine (for example, SEQ ID NO: 83-93) have been developed, each of which was able to protect mice against disseminated candidiasis with the survival rate of 80-100% up to 120 days post challenge.

Subsequent data demonstrates antibodies specific for universal peptides (such as, GPV-MO1, GPV-M1, GPV-M2) are each capable of protecting the animals. Universal peptide-related monoclonal antibodies will be isolated that can be used for immediate protection by passive administration of MAbs, which represents strong alternative measures to conventional antifungal drug therapy.

Without wishing to be bound by theory, embodiments as described herein broadens the range of the protection beyond that of just Candida albicans to include protection against fungal species of medical significance, including but not limited to additional Candida spp. as well as Aspergillus spp.

Embodiments as described herein comprise universal synthetic peptide vaccine and double-epitope universal peptide vaccine protective against invasive fungal infection. Without wishing to be bound by theory, the double-peptide vaccine formulation offers protective duality against all medically important Candida spp., with the advantages of being cheaper, easier to synthesize, and a more practical application as compared to the original glycopeptide formula (Xin, Hong, et al. “Self-adjuvanting glycopeptide conjugate vaccine against disseminated candidiasis.” PLoS One 7.4 (2012): e35106, which is incorporated by reference herein in its entirety).

Embodiments as described herein can further comprise coupling, such as covalently linking, the peptide or multi-peptide vaccines to a protein carrier, non-limiting examples of which comprise tetanus toxoid (TT), bovine serum albumin (BSA), ovalbumin, or keyhole limpet hemocyanin (KLH). In some instances, peptide antigens can be too small (for example, less than 7 amino acids in certain instances) to generate significant immune responses on their own. A longer peptide, such as those longer than 20 amino acids, increases the peptide's immunogenicity, but also increases the peptide's chances of cross-reactivity. On the other hand, a shorter peptide, such as those 10 amino acids or less, may improve the specificity, but may not be immunogenic. To solve this problem, these small peptides can be conjugated to larger carrier proteins, such as bovine serum albumin (BSA), ovalbumin, or keyhole limpet hemocyanin (KLH). One of the advantages of KLH is that it does not interfere with ELISA or western blotting because it is not used as a blocking reagent. One common means of conjugation method is the maleimide method, which couples the cysteine residue of the peptide to the carrier protein. For example, see US20140271650, which is incorporated by reference herein in its entirety). To perform this conjugation, one cysteine residue is added to the N- or C-terminus of the peptide so that it may be linked to the carrier protein.

Such embodiments can be an acceptable vaccine formulation for use in a subject, such as a human.

Collectively, embodiments as described herein comprising universal peptide vaccines, such as those comprising double or multiple universal peptide epitopes, can provide broader immune recognitions and subsequent induction of antibody-mediated protection against disseminated fungal infections, such as disseminated candidiasis, and other fungal infections, such as aspergillosis infection.

Compared with Existing Candida Vaccine

Several vaccines targeting Candida have been described in preclinical settings (De Bernardis, F. et al. A virosomal vaccine against candidal vaginitis: immunogenicity, efficacy and safety profile in animal models. Vaccine 30, 4490-4498 (2012) and Schmidt, C. S. et al. NDV-3, a recombinant alum-adjuvanted vaccine for Candida and Staphylococcus aureus is safe and immunogenic in healthy adults. Vaccine 30, 7594-7600 (2012), each of which are incorporated by reference herein in their entireties). For example, a protein conjugate vaccine consisting of laminaran (algal glucan) linked to diphtheria toxoid as a carrier protein resulted in significant protection against disseminated candidiasis. However, although carbohydrate antigens are important immune targets associated with a variety of pathogens, most carbohydrates are intrinsically T cell-independent antigens which diminishes their efficacy as immunogens. Further limiting the use of carbohydrate-based vaccines is the complexity of oligosaccharide synthesis. Embodiments as described herein overcome this limitation as the peptide-based vaccines greatly simplify the development of new anti-fungal vaccines, with improved safety, purity, and standardization (Peptide-Based Immunotherapeutics and Vaccines, Journal of Immunology Research Volume 2014 (2014), Article ID 256784, 2 pages; and Skwarczynski, Mariusz, and Istvan Toth. “Peptide-based synthetic vaccines.” Chemical Science 7.2 (2016): 842-854, each of which are incorporated herein by reference in their entireties).

Another example of a Candida vaccine previously described is based on the agglutinin-like sequence (Als) family of proteins from Candida albicans. Specifically, the recombinant N-termini of the C. albicans surface adhesins Als1p or Als3p (rAls1p-N or rAls3p-N) protected mice from otherwise lethal disseminated candidiasis, and also reduced fungal burden in a vaginitis model and a steroid-treated oropharyngeal candidiasis model. However, the Als-vaccine only protected disseminated candidiasis caused by a single Candida species, Candida albicans, and offered no protection against other medically important candidiasis and Aspergillus species (Schmidt, Clint S., et al. “NDV-3, a recombinant alum-adjuvanted vaccine for Candida and Staphylococcus aureus, is safe and immunogenic in healthy adults.” Vaccine 30.52 (2012): 7594-7600). Unlike the Als-vaccine, embodiments as described herein can protect against disseminated candidiasis caused by diverse medically important fungal species, non-limiting examples of which include Candida spp such as C. albican, C. glabrata, C. tropicalis, C. parapsilosis, C. krusei, Candida dubliniensis, Candida auris, and C. parapsilosis, and Aspergillus spp such as A. fumigatus, A. flavus, A. niger, and A. terreus, A. lentulus.

Compared with Existing Aspergillus Vaccines

The feasibility of vaccination of mice with crude antigen preparations from an Aspergillus strain, A. fumigatus, has been demonstrated (Cenci E, Mencacci A, Bacci A, Bistoni F, Kurup V P, Romani L. T cell vaccination in mice with invasive pulmonary aspergillosis. J Immunol. 2000; 165:381-8; Ito J I, Lyons J M. Vaccination of corticosteroid immunosuppressed mice against invasive pulmonary aspergillosis. J Infect Dis. 2002; 186:869-71. doi: 10.1086/342509). However, a crude protein preparation is not going to support a clinical development program as a defined antigen preparation that can be manufactured to GMP-compliance must be identified. Thus, a defined antigen preparation must be identified. Embodiments as described herein, such as the peptide-based vaccines, can be manufactured to GMP-compliance ideal for clinical development and clinical use.

Subcutaneous vaccination of mice with recombinant Asp f 3, or specific fractions thereof, protected mice from subsequent lethal inhalational challenge with A. fumigatus (Ito J I, Lyons J M, Hong T B, Tamae D, Liu Y K, Wilczynski S P, Kalkum M. Vaccinations with recombinant variants of Aspergillus fumigatus allergen Asp f 3 protect mice against invasive aspergillosis. Infect Immun. 2006; 74:5075-84). While protection with the soluble form of protein required the use of TiterMax adjuvant (which is too toxic for use in humans), a protein precipitate form of the vaccine administered as a suspension in methylcellulose carrier was also protective and thus potentially clinically relevant. However, compared to the vaccine components of protein mixtures as described in Ito et al., embodiments as described herein, such as the peptide or double-peptide vaccines, are much safer and cheaper to manufacture; with guaranteed purity and consistency in synthesis. Ito et al. discussed the use of crude protein mixtures, which would not be suitable for use in humans due to safety concerns related to toxicity and allergenicity. Without wishing to be bound by theory, embodiments as described herein, including peptide vaccines and peptide vaccines with modifications (such as linking to carrier proteins, mixtures with FDA approved adjuvants, for example), are suitable for human use, such as a vaccine against invasive pulmonary aspergillosis.

### Advantages of Double/Multiple Universal Peptide Vaccines

Univalent vaccines comprise a single recombinant protein antigen. Although such C. albicans vaccines can generate immune memory responses that lead to the production of virulence-neutralizing, protective antibodies, they are unsuitable to achieve high-grade, persistent protection in humans due primarily to their univalence.

A defining characteristic of C. albicans is its extraordinary range of virulence factors that facilitate tissue invasion by enabling the fungus to escape from host immunity. Taking advantage of this defining characteristic, embodiments as described herein comprise multivalent vaccines that targets two or more concurrent but unrelated virulence factors which, without wishing to be bound by theory, will have a better chance of providing protection against candidiasis. Combination of antigens that are related to key C. albicans virulence attributes or biological functions can induce additive or synergistic immune responses, broadening the spectrum of protective antibodies and reducing the probability of fungal immune evasion. As an example of such a multivalent vaccine, the double-peptide vaccine GPV-M1-KK-GPV-P2 comprises the GPV-P2 peptide from Pgk1 protein and the GPV-M1 peptide from Met6 protein, both of which are cell wall proteins of Candida spp.

### Continued Development

A detailed mechanistic understanding of universal peptide vaccines protection is contemplated, which will elucidate the fundamental requirements of host defense against disseminated candidiasis.

The protective efficacy of embodiments as described herein, such as universal peptides and double peptides, in combination with tetanus toxoid carrier protein (TT) is contemplated, as TT has been widely used in US Food and Drug Administration (FDA)-approved vaccines.

A dosing schedule, route of administration, and use of an adjuvant are contemplated, as such information can support an “Investigational New Drug” (IND) application to support the commencement of clinical testing.

### Example 3

### Abstract

Fully synthetic universal peptide vaccines (Table 1) and double-peptide vaccines (Table C) against Candida spp cell surface epitopes have been designed and subsequently tested for their ability to protect mice against disseminated candidiasis. For example, a panel of peptides have been identified that are universally produced by all medically important Candida species including but not limited to C. albicans, as well as by some Aspergillus species. Such universal peptide/double-peptide vaccines as described herein are expressed at high homology (81-100%) in many medically important fungi, including but not limited to medically important Candida spp (Table 2), Aspergillus fumigates, Aspergillus niger and other Aspergillus spp. For example, Table 1 lists nine fully synthetic universal peptide vaccines, each of which can protect mice against disseminated candidiasis as demonstrated by survival rate of 80-100% up to 120 days post challenge.

Further, monoclonal antibodies (mAbs), such as IgG monoclonal antibodies, specific for these universal peptides were isolated and purified (Table 3), and tested for protective efficacy by passive transfer in mouse model of human disseminated candidiasis. Each mAb can protect naïve mice against invasive candidiasis by passive transfer of the mAb before lethal challenge. The synergy of protective efficacy with two mAbs combination have also been tested (See Table 8).

Collectively, C. albicans cell surface peptide-related and Candida universal peptide-related monoclonal antibodies (mAbs) that can be used for immediate protection by passive administration of those mAbs to naïve animals have been isolated, which represents attractive alternatives to conventional antifungal drug therapy. Without wishing to be bound by theory, mAbs specific for the universal peptide vaccines are protective in mouse model of disseminated candidiasis caused by non-albicans Candida species, non-limiting examples of which comprise C. glabrata, C. parapsilosis, C. tropicalis, C. dubliniensis, C. krusei, and C. auris.

Embodiments as described herein further comprise double chimeric peptide vaccine constructs, such as those that are further modified to be suitable for human use by simple i.p. immunization approach. Without wishing to be bound by theory, such embodiments can induced high levels of protection without the need for an adjuvant and carrier protein, as demonstrated by simple i.p. immunization of mice with the peptide. Humans have a much more robust immune system as compared to mice, so we expect the newly designed double-peptide constructs, with appropriate spacer amino acids and T cell universal epitopes, can induce protective immunity under adjuvant-free conditions, via a simple i.p. injection, which leading to a new peptide vaccine formula with immunization approaches perfectly feasible for human use.

### Introduction

Morbidity and mortality from invasive fungal infections remain unacceptably high despite availability of new antifungal agents, underscoring the need for more effective preventative strategies. Invasive fungal diseases often takes hold when a subject's natural defenses are weakened. These infections frequently occur in hospital settings. Vaccination of high-risk groups is a particularly promising strategy to prevent invasive fungal infections because easily identifiable risk factors are clearly defined for many such infections, non-limiting examples of which comprise candidiasis and aspergillosis. Often, development of such risk factors precedes infection, affording a wide window of opportunity to vaccinate acutely at-risk patients before the onset of infection.

Being a main invasive fungal disease, hematogenously disseminated candidiasis in humans is the leading cause of nosocomial bloodstream infection in the US. Aspergillus is the second most common cause of nosocomial, invasive fungal infections, with an incidence of approximately 5 per 100,000 population in the United States. Embodiments as described herein can protect and/or prevent infections caused by Candida spp. and Aspergillus spp., the latter of which has an extremely high mortality rate as compared to the former despite antifungal therapy (45% to >80%).

The most common causes of invasive fungal infections are members of the genus Candida (1). Disseminated candidiasis is the cause of more fatalities than any other systemic mycosis (2,3). The medically significant Candida species that cause more than 90% of invasive infections in humans include: C. albicans, the most common species identified (˜60%); C. glabrata (15-20%), C. parapsilosis (10-20%), C. tropicalis (6˜12%); and C. krusei (<5%) (4). Vaccination of high-risk groups is a strategy to prevent invasive Candida infection (5). Reports highlight the development of peptide and glycopeptide vaccines and chimeric double-peptide vaccine against C. albicans cell surface epitopes that can induce protection in mice against disseminated candidiasis (6-9). Importantly, the reported peptides only offer protection against C. albicans, and are unlike embodiments as described herein which offer protection against infections caused by both Candida spp. and other fungal species such as Aspergillus sp. It was also demonstrated that mAbs specific for the peptides protect both immunocompetent and neutropenic mice against Candidiasis (7,9,10).

Subsequently, double-peptide vaccines designed for human use induced high levels of protection without the need for an adjuvant and carrier protein via the simple i.p. immunization of mice with the peptide (Xin, H. “Double chimeric peptide vaccine and monoclonal antibodies that protect against disseminated candidiasis.” J Vaccines Vaccine 5 (2014)).

To provide for broader coverage against all medically important non-albicans Candida (NAC) species in addition to C. albicans, universal peptide vaccines as described herein were designed and mAbs were isolated, both of which were tested for the protective efficacy against disseminated candidiasis in mice.

Without wishing to be bound by theory, the double-peptide vaccine formulation demonstrates protective duality against all medically important Candida spp. while providing for a relatively lost cost to manufacture and easier synthesis. Our work demonstrates that development of a double chimeric peptide vaccine that induces protective immune responses against two unrelated cell surface peptide epitopes of C. albicans is a better approach against the disease, for example than those that induce a protective immune response against a single epitope. For example, the double vaccine can protect against the mutant strain that may lose one of the epitopes. In addition, combinations of multiple MAbs can provide the most effective form of passive transfer protection, which can demonstrate emergent properties with regard to protective efficacy.

The term “emergent properties” can refer to a property that cannot be explained by the individual components alone, and usually reflects an outcome that is greater than the sum of the parts with a certain form of novelty. As described herein, for example, combinations of mAbs, as opposed to each individual antibody alone, can synergistically benefit the host, such as providing greater protection against fungal infections than the sum of each mAb alone, requiring much less dose of mAb for protective effects, offer complete protection (i.e., 100% survival), and result in fungal cells being completely cleared from organs.

As described herein, the universal peptide vaccines, which comprise for example double or multiple universal peptide epitopes, provide for universal immune recognitions and induction of antibody-mediated protection against disseminated candidiasis against aspergillosis infection.

Of concern when developing potential Candida vaccines is the notion that candidiasis occurs almost exclusively in immune-compromised patients whose immune system may not be equipped to respond immunologically to a vaccine. However, there is extensive literature confirming the immunogenicity and efficacy of vaccines even in patients with weakened immune systems—for example, those with neutropenia, active leukemia, HIV infections, or those receiving immunosuppressive corticosteroids (dos Santos, Sigrid De Sousa, et al. “Haemophilus influenzae type b immunization in adults infected with the human immunodeficiency virus.” AIDS research and human retroviruses 20.5 (2004): 493-496; Dockrell, David H., et al. “Immunogenicity of three Haemophilus influenzae type b protein conjugate vaccines in HIV seropositive adults and analysis of predictors of vaccine response.” Vaccine 17.22 (1999): 2779-2785; Levin, Myron J., et al. “Immunization of HIV-infected children with varicella vaccine.” The Journal of pediatrics 139.2 (2001): 305-310; Tedaldi, Ellen M., et al. “Hepatitis A and B vaccination practices for ambulatory patients infected with HIV.” Clinical Infectious Diseases 38.10 (2004): 1478-1484; Sinisalo, Marjatta, et al. “Haemophilus influenzae type b (Hib) antibody concentrations and vaccination responses in patients with chronic lymphocytic leukaemia: predicting factors for response.” Leukemia & lymphoma 43.10 (2002): 1967-1969. Nordoy, Tone, et al. “Cancer patients undergoing chemotherapy show adequate serological response to vaccinations against influenza virus and Streptococcus pneumoniae.” Medical Oncology 19.2 (2002): 71-78; Leung, Ting-Fan, et al. “Immunogenicity of a two-dose regime of varicella vaccine in children with cancers.” European journal of haematology 72.5 (2004): 353-357; Klugman, Keith P., et al. “A trial of a 9-valent pneumococcal conjugate vaccine in children with and those without HIV infection.” New England Journal of Medicine 349.14 (2003): 1341-1348; Madhi, Shabir A., et al. “The impact of a 9-valent pneumococcal conjugate vaccine on the public health burden of pneumonia in HIV-infected and -uninfected children.” Clinical infectious diseases 40.10 (2005): 1511-1518). At the same time, the development of effective mAbs is extremely important because they can protect against severe infection more rapidly than antifungal drugs, and they are also effective against antifungal resistance in multiple Candida species. As described herein. peptide-vaccine-specific monoclonal antibodies (mAbs) for the passive immunization in host against Candida infection (Table 3 and Table 4) have been isolated and purified. Further, the combination of two protective mAbs into one cocktail provides for a more effective protection as compared to single mAb treatment (Table 6 and Table 7).

### Embodiments in this Example

Embodiments as described herein comprise antigenic universal peptides, such as universal peptides, that are expressed by clinically related Candida species in addition to C. albicans. Without wishing to be bound by theory, such peptides can protect against all medically significant Candida spp.

Embodiments as described herein also comprise multivalent peptide vaccines that are superior to the existing one-antigen (univalent) vaccine. According to Cassone's 2013 nature review (Cassone, Antonio. “Development of vaccines for Candida albicans: fighting a skilled transformer.” Nature Reviews Microbiology 11.12 (2013): 884-891), it is unlikely that a univalent vaccine will be successful as the complexity of C. albicans antigens and host-invasion mechanisms makes it difficult to obtain effective immune protection with a single antigen. Our multivalent vaccine that targets two or more unrelated virulence factors will have a better chance of providing protection and avoiding immune evasion. Without wishing to be bound by theory, multivalent vaccines as described herein which targets two or more unrelated protective epitopes offer improved protection when compared to univalent peptide vaccines and can avoid immune evasion.

Embodiments as described herein further comprise mAbs and protective combinations thereof which can protect against severe infection more rapidly than antifungal drugs, and are also effective against antifungal resistance in multiple Candida species.

Embodiments as described herein further comprise double peptide vaccine, further including such vaccines that do not require an adjuvant and carrier protein in order to be effective. Peptide-based vaccines are promising approaches to treat infections, however their sometimes-weak immunogenic potency impedes their clinical application. Addressing this unmet need are embodiments as described herein comprising double-peptide construct. Such embodiments can comprise spacer amino acids and/or T cell universal epitopes, and can induce protective immunity under adjuvant-free conditions, leading to a peptide vaccine, compositions comprising the same, and methods of using the same for immunizations of mammals, such as humans.

### Materials and Methods

Candida strains and culture conditions. C. albicans 3153A and SC5314, C. parapsilosis (ATCC MYA-4646), C. tropicalis (ATCC 28775), C. krusei (ATCC 6258), C. glabrata (ATCC 2001) and S. cerevisiae (ATCC 9463) were grown as stationary-phase yeast cells in glucose-yeast extract-peptone broth at 37° C., washed and suspended to the appropriate cell concentration (5×106/ml) in Dulbecco's PBS (DPBS; Sigma), and used to infect mice intravenously (i.v.) as described before (14,15). C. albicans strain 3153A was also used for serum antibody absorption, immunofluorescence staining and flow cytometric analysis.

Mice. BALB/c and C57BL/6 female mice (National Cancer Institute Animal Production Program, Frederick MD) 5 to 7 weeks old were used throughout. Mice were always maintained in our AAALAC-certified animal facility and all animal experiments were done in accordance with a protocol approved by the Institutional Animal Care and Use committee (IACUC) at LSU Health Sciences Center (LSUHSC).

Peptide vaccines. Two 14-mer peptides Fba and Met6 are derived from N-terminus of C. albicans cell wall proteins fructose-bisphosphate aldolase (Fba) and methyltetrahydropteroyltriglutamate (Met6). Fba peptide (YGKDVKDLFDYAQE (SEQ ID NO: 95)) and Met6 peptide (PRIGGQRELKKITE (SEQ ID NO: 96)) were produced commercially (GenScript). All the universal peptides, derived from N-terminus of Candida cell wall proteins, were listed at Table 1 with sequence detail. They were produced commercially (GenScript).

Protective mAbs. Hybridoma clones, which produce mAbs specific for Fba peptide, Met6 peptide, Hwp1 peptides and the universal peptides (Table 3 and Table 4) were generated from mice vaccinated with peptide-pulsed dendritic cell (DC) preparation as described previously (6). Briefly, BALB/c mice were immunized by injection of synthetic peptide pulsed DCs to stimulate the production of antibodies against peptide as described above. Ten days after the second booster, serum was taken from each animal to determine animals with the highest anti-peptide titers for subsequent sacrificing, removal of spleens and preparation of single cell suspensions. Hybridoma clones were established by the polyethylene glycol facilitation of fusion of spleen cells to an SP2/0-AG14 myeloma cell line by standard protocols. Hybridoma clones were screened by ELISA for production of specific anti-peptide antibody; only the highest titers and most rapidly growing clones were selected for subsequent cloning x3 or more by limiting dilution.

The hybridoma cell lines were initially grown in antibiotic-free RPMI 1640 medium (Sigma) supplemented with 10% fetal bovine serum (Invitrogen) and 2 mM L-glutamine (Sigma) at 37° C. and in the presence of 5% CO2. For antibody production, the hybridoma clones were grown in antibiotic-free, BD cell mAb serum-free medium (but containing 1.1 mg bovine serum albumin/ml) in a CELLine device (BD, Bedford, MA). All the peptide-specific mAbs (IgGs) were purified and analyzed as described before (9). In short, the supernatant was collected and mAb was purified by affinity chromatography using a Protein A Sepharose 4FF column (GE Healthcare, USA). The isotype of mAb was determined with a Mouse Monoclonal Antibody Isotyping Kit (Pierce, USA).

Isolation and culture of dendritic cells (DCs) from mouse bone marrow. Dendritic cells (DCs) were generated from mouse bone marrow by a previously described method (6,16). Briefly, donor mice were euthanized by CO2 asphyxiation, their long bones and tibias were aseptically removed, bone marrow was flushed from the bones by forcibly injecting several ml of RPMI-1640 and clumps were removed or dispersed by gentle pipetting through a sterile 70-mm cell strainer. Red blood cells were lysed (ACK lysing buffer, 0.15 M NH₄Cl, 1.0 mM KHCO3, 0.1 mM EDTA) for 4 min and the remaining bone marrow cells were suspended in complete medium [CM, RPMI-1640 supplemented with 10% FBS (FBS), 2 mM L-glutamine, 1% of nonessential amino acids and 100 units/ml penicillin and 100 μg/ml streptomycin], adjusted to 2×105 cells per ml plated in 6-well plates at 5 ml per well and cultured for up to 9 days in the presence of 40 ng/ml of rmGM-CSF and rmIL-4 (R&D Systems) at 37° C., 5% CO2. On days 4 and 7 of culture, the same amount of fresh GM-CSF and IL-4 was added to the wells.

Active Immunizations with peptide pulsed dendritic cells. The antigen-pulsed DC immunization approach proved to be a powerful way to identify the protective peptide epitopes on C. albicans cell surface (6,10,17). We have demonstrated the feasibility of this approach in our finding of protective peptide-based vaccines (7). It allows us to perform rapid throughput evaluation of candidate short peptides. When protective double universal peptide conjugate(s) become identified by this study, immunization approaches suitable for human use will be applied in further studies. All active vaccinations were conducted as previously described (6,7,9). DCs were pulsed in vitro with universal peptide as described before with minor modification by totally removing CFA for last booster (6). Briefly, DCs in culture were pulsed with the peptide antigen (1 μM) on day 6. On day 7, PGE2 (10-7M) was added along with LPS (2 μg/ml, Sigma) for 24h to induce DC maturation. On day 9, antigen-pulsed DCs were washed extensively and 5×105 in 200 μl DPBS were given by intraperitoneal (i.p.) route as the priming dose to mice. The mice were boosted i.p. at day 14 and day 28 with fresh antigen-pulsed DCs without adjuvant.

To test the efficacy of the vaccine in immunocompromised mice, vaccinated mice were induced neutropenia by intraperitoneal injection of 200 mg/kg of cyclophosphamide (CY; Sigma-Aldrich) on day −3 followed by another 4 doses (150 mg/kg) every 10 days on days 10, 20, 30 and 40 relative to infection.

Induction of neutropenia. Although C. albicans remains the dominant disease-causing pathogen of this genus, rates of infections caused by non-albicans Candida (NAC) species are increasing. Among these Candida species, only C. albicans is pathogenic in the normal mouse model. To test vaccine and mAbs against non-albicans Candida spp, we have developed neutropenic murine models of disseminated infection by C. glabrata, C. tropicalis, and C. parapsilosis. Before challenge, BLAB/c mice were made neutropenic by intraperitoneal receipt of a 200 mg/kg dose of cyclophosphamide (CY; Sigma-Aldrich) at day −3. The i.p. injection (150 mg/kg) is repeated every 10 days after infection (i.e., at days 10, 20, 30, and 40 post-infection) in order to maintain low neutrophil counts for the entire experimental procedure. The experiment was set up to test this regimen, which has been shown to render mice neutropenic (The absolute neutrophil count is <500 cells/mm3) within 3-4 day of the first cyclophosphamide injection, and neutropenia lasts until the termination of the experiments (day 50). To evaluate leukocytopenia in these mice, blood samples were collected from tail veins 3 days after each CY or saline injection (n=10 in each group). Total leukocyte and differential cell counts were determined on a hemocytometer and by Wright-Giemsa staining. Body weights of the mice were also measured and compared for 14 days after the first CY or saline injection (n=10 in each group).

Fungal challenge dose and assessment of protection. Although C. albicans remains the dominant disease-causing pathogen of this genus, rates of infections caused by non-albicans Candida (NAC) species are increasing (18). Among these Candida species, only C. albicans is pathogenic in the normal mouse model (19,20). We have developed neutropenic murine models of disseminated infection by C. glabrata, C. tropicalis, and C. parapsilosis. Preliminary experiments demonstrated the optimal dose of each Candida strain for producing an acute infection, with 80-100% of animals dying within 10-15 days. In details, mice of different experimental groups (five mice per group) were intravenously infected with 5×106 viable C. parapsilosis ATCC MYA-4646 (A), 1×10⁸ C. glabrata ATCC 2001 cells (B) and 1×107 C. tropicalis ATCC 28775 cells (C) in 0.1 ml DPBS. As controls, immunocompetent mice were challenged with the same dose of each Candida strain tested.

Passive transfer of MAbs by intraperitoneal (i.p.) route. The preventive effect of peptide-specific mAbs listed in Table 3 and Table 4 in naïve mice was examined by passive transfer experiments. Each mAb was appropriately diluted in DPBS to give a 40,000-100,000 ELISA titer against each corresponding specific peptide coated on the plate. For testing, mice received 0.5 ml of each mAb or 1 ml of two mAbs in combination (0.5 ml of each) intraperitoneally. Table 6 and Table 7 have listed all the peptide-specific mAb combinations that have been tested, as well as the protective efficacy of each mAb cocktail as compared to single mAb treatment. The negative control materials tested in mice were single mAb or DPBS. Control mice received 0.5 ml of the DPBS diluents or single mAb treatment. For each condition, 6- to 8-week-old female BALB/c mice (NCI) were given 0.5 ml of test mAb, or 1 ml two mAb cocktails or control materials intraperitoneally, followed 4 h later by 0.1 ml intravenously of defined lethal dose of yeast cells per milliliter of DPBS. Mice were divided into groups with five mice each and three independent experiments were carried out. In some experiments, mice were given the same dose of mAbs or control materials on every other day post-challenge for two weeks, as compared to the protective efficacy of mAb in combinations. All mice were sacrificed on day 50. For animal groups that were challenged non-albicans candida species, determined optimal challenge doses were applied for each Candida strain.

Statistical Analysis. Survival times were statistically evaluated by Kaplan-Meier (GraphPad Prism, version 6), and statistical significance was subsequently calculated for each preset time point of analysis. A P value <0.05 was considered to be statistically significant.

### Results:

Table 2 lists universal peptide vaccines that induced protection against disseminated candidiasis caused by C. albicans in mice. The universal peptide vaccines listed in Table 1 are expressed at high homology by all analyzed medically important Candida species in addition to C. albicans (see details at Table 2). By dendritic cell (DC) based immunization approach as described herein, each of the universal peptide vaccines was able to protect mice against disseminated candidiasis caused by C. albicans, with the survival rate 80-100% up to 80-120 days post challenge.

TABLE 2

| Universal | | SEQ |
|---|---|---|
| peptide | | ID |
| vaccines | Sequences | NO: |
| GPV-M1 | cFWVNPDCGLKTR | 1 |
| GPV-M2 | cTTTIGSFPQTKDIR | 2 |
| GPV-M3 | ADKDSLDLEPISLLPK | 3 |
| GPV-M4 | YNLPLFPTTTIGSFPQTKDIR | 4 |
| GPV-M5 | DDVSGKIQALQLGLALR | 5 |
| GPV-M6 | GWPEVKESLTNMVEAAK | 6 |
| GPV-M7 | YTKFDLAPIDVLFAMGR | 7 |
| GPV-P1 | cAILGGSKVSDKI | 14 |
| GPV-P3 | cIVIIGGGDTATVAKK | 16 |

Table 3 displays the homology of protective universal peptide vaccines among all medically significant Candida species.

TABLE 3

| Candida spp. | GPV-P1 | GPV-P3 | GPV-M1 | GPV-M2 | GPV-M3 | GPV-M4 | GPV-M5 | GPV-M6 | GPV-M7 |
|---|---|---|---|---|---|---|---|---|---|
| albicans | 100% | 100% | 100% | 100% | 100% | 100% | 100% | 100% | 100% |
| glabrata | 100% | 100% | 100% | 100% | 81% | N/A | N/A | N/A | 82% |
| parapsilosis | 100% | 100% | 100% | 100% | 100% | 100% | 88% | 94% | 100% |
| tropicalis | 100% | 100% | 100% | 100% | 100% | 100% | 100% | 94% | 94% |
| dubliniensis | 100% | 100% | 100% | 100% | 100% | 100% | 100% | 100% | 100% |
| krusei | 100% | 100% | 100% | 100% | 100% | 100% | N/A | 0% | N/A |
| auris | N/A | N/A | N/A | 100% | 93% | 100% | 82% | 82% | 100% |

Table 4 displays hybridoma clones producing universal peptide-specific mAbs and the isotype of each protective mAbs. The hybridoma clones that produce the universal peptide specific mAbs listed above were generated from mice vaccinated with peptide-pulsed dendritic cell (DC) preparation as described previously (6). The isotype of mAb was determined with a Mouse Monoclonal Antibody Isotyping Kit (Pierce, USA). All the mAbs listed at Table 4 were able to confer enhanced protection against systemic candidiasis in passive transfer experiments in mouse model of human disseminated candidiasis. In detail, BALB/c mice were given an i.p dose of each mAb four hours before hematogenous challenged with a lethal dose of C. albicans 3153A cells. Mice that received mAb treatments had prolonged survival as compared to control animals, which received DPBS buffer or adsorbed mAb solutions at the same time with mAb treatment. In addition, surviving animals that received the antibody had significantly reduced or non-detectable fungal burdens in their kidneys as compared to controls. Importantly, passive protection was prevented by removal of the mAbs by absorption with Candida cells before transfer, which provided strong additional evidence for the protection being due to the mAb.

In embodiments, the antibody or fragment thereof produced by the clones generated in response to an antigenic universal peptide (e.g, those hybridoma clones listed in Table 4) comprises a variable domain having a variable light chain (VL) amino acid or nucleotide sequence at least 90% identical to those produced by the clones generated in response to an antigenic universal peptide or chimeric peptide, or having a variable heavy chain (VH) amino acid or nucleotide sequence at least 90% identical to those produced by the clones generated in response to an antigenic universal peptide or chimeric peptide. For example, the antibody or fragment thereof comprises a variable domain having a variable light chain (VL) amino acid or nucleotide sequence at least 90%, 91%, 92%, 93%, 94%, 95%, 96%, 97%, 98%, 99%, or 100% identical to those of the clones detailed in TABLE 4 or having a variable heavy chain (VH) amino acid or nucleotide sequence at least 90% identical to those of the clones detailed in TABLE 4. In embodiments, the variable light chain (V_{L}) and/or variable heavy chain comprises fully human framework region(s).

TABLE 4
Hybridoma clone and related protective
peptide-specific mAbs

| Hybrid- | | mAb | peptide | peptide |
|---|---|---|---|---|
| omas | mAb | isotype | vaccine | Sequences |
| 2C9G3 | 2C9 | IgG2a | GPV-M2 | cTTTIGSFPQTKDIR |
| | | | universal | (SEQ ID NO: 2) |
| 5A9D1 | 5A9 | IgG3 | GPV-M2 | cTTTIGSFPQTKDIR |
| | | | universal | (SEQ ID NO: 2) |
| 5A11B1 | 5A1 | IgG3 | GPV-M2 | cTTTIGSFPQTKDIR |
| | | | universal | (SEQ ID NO: 2) |
| 6E3D9 | 6E3 | IgG1 | GPV-M1 | cFWVNPDCGLKTR |
| | | | universal | (SEQ ID NO: 1) |
| 1D4H5 | 1D5 | IgG1 | GPV-M1 | cFWVNPDCGLKTR |
| | | | universal | (SEQ ID NO: 1) |
| 7A3C4 | 7A3 | IgG1 | GPV-M1 | cFWVNPDCGLKTR |
| | | | universal | (SEQ ID NO: 1) |
| 10E7E2 | 10E7 | IgG1 | GPV-P3 | cIVIIGGGDTATVAK |
| | Or | | universal | K (SEQ ID NO: |
| | 10E7E2 | | | 16) |
| 1A11H8 | 1A11 | IgG3 | GPV-P1 | cAILGGSKVSDKI |
| | | | universal | (SEQ ID NO: 14) |
| 10B6H8 | 10B6 | IgG1 | GPV-P1 | cAILGGSKVSDKI |
| | | | universal | (SEQ ID NO: 14) |
| 5G5G3 | 5G5 | IgG2a | GPV-P1 | cAILGGSKVSDKI |
| | | | universal | (SEQ ID NO: 14) |

The underlined represents the peptide antigen (can also be referred to as peptide epitope) that is recognized and bound by the antibody, such as the monoclonal antibody.

Table 5 displays the hybridoma clones producing protective mAbs that are specific for peptides derived from C. albicans cell wall proteins and the isotype of each protective mAbs. Three T-cell peptides (Fba peptide, Met6 peptide and Hwp1 peptide) found in Candida albicans cell wall proteins were selected by algorithm peptide epitope searches and fully synthetic peptides were used to immunize mice by DC-based immunization approach. The three cell wall proteins were selected because of expression during human candidiasis and cell wall association, and included fructose-bisphosphate aldolase (Fba); methyltetrahydropteroyltriglutamate--homocysteine methyltransferase (Met6); hyphal wall protein-1 (Hwp1). The hybridoma clones listed at Table 5, which produce the peptide specific mAbs, were generated from mice vaccinated with peptide-pulsed dendritic cell (DC) preparation as described previously (6). The isotype of mAb was determined with a Mouse Monoclonal Antibody Isotyping Kit (Pierce, USA). All the mAbs listed at Table 5 conferred enhanced protection against systemic candidiasis in passive transfer experiments in mouse model of human disseminated candidiasis. In detail, BALB/c mice given an i.p dose of each mAb four hours before hematogensous challenge with a lethal dose of C. albicans 3153A had prolonged survival as compared to control animals. In addition, surviving animals that received the antibody had reduced/non detectable fungal burden in their kidneys. Importantly, passive protection was prevented by removal of the mAbs by absorption with Candida cells before transfer, which provided strong additional evidence for the protection being due to the mAb).

TABLE 5
Hybridoma clone, peptide vaccines, and
related protective peptide-specific mAbs

| Hybrid- | | mAb | Peptide | Peptide |
|---|---|---|---|---|
| omas | mAbs | isotype | vaccine | sequences |
| 3H7E3 | 3H7 | IgG1 | Met6 | PRIGGQRELKKITEc |
| | | | peptide | (SEQ ID NO: 30) |
| 5E12D10 | 5E12 | IgG1 | Met6 | PRIGGQRELKKITEC |
| | | | peptide | (SEQ ID NO: 30) |
| 2B10C1 | 2B10 | IgG1 | Met6 | PRIGGQRELKKITEc |
| | | | peptide | (SEQ ID NO: 30) |
| 7H6A2 | 7H6 | IgG2b | Met6 | PRIGGQRELKKITEc |
| | | | peptide | (SEQ ID NO: 30) |
| 6H1G8 | 6H1 | IgG2b | Hwp1 | QGETEEALIQKRSYc |
| | Or | | peptide | (SEQ ID NO: 31) |
| | 6H1G8 | | | |
| 3B5H7 | 3B5 | IgG2b | Hwp1 | QGETEEALIQKRSYc |
| | | | peptide | (SEQ ID NO: 31) |
| 5B11H1 | 5B11 | IgG1 | Hwp1 | QGETEEALIQKRSYc |
| | | | peptide | (SEQ ID NO: 31) |
| 2D5F7 | 2D5 | IgG2b | Fba | YGKDVKDLFDYAQEc |
| | | | peptide | (SEQ ID NO: 29) |
| 3E2G2 | 3E2 | IgG2b | Fba | YGKDVKDLFDYAQEc |
| | | | peptide | (SEQ ID NO: 29) |
| 6G9E11 | 6G9 | IgG2b | Fba | YGKDVKDLFDYAQEc |
| | | | peptide | (SEQ ID NO: 29) |
| 9C11F6 | 9C11 | IgG2b | Fba | YGKDVKDLFDYAQEc |
| | | | peptide | (SEQ ID NO: 29) |
| 7H5H6 | 7H5 | IgG2b | Fba | YGKDVKDLFDYAQEc |
| | | | peptide | (SEQ ID NO: 29) |
| 7C6E8 | 7C6E8 | IgG3 | Pgk1 | cVPLDGKTITNNQRI |
| | | | peptide | (SEQ ID NO: 236) |
| 9F2G5 | 9F2G5 | IgG2a | Pgk1 | cVPLDGKTITNNQRI |
| | | | peptide | (SEQ ID NO: 237) |

Table 6 displays protective universal-peptide-specific mAbs that have been tested in mouse model of human disseminated candidiasis caused by C. albicans, C. tropicalis and C. glabrata. The preventive effect of each universal peptide-specific mAb was examined by passive transfer experiments. Each mAb was appropriately diluted in DPBS (0.2 g/l) to give a 100,000 ELISA titer against each universal peptide coated on the plate. For testing, 5 mice of each tested group received 0.5 ml of each mAb intraperitoneally. The negative control materials tested in mice were mAbs absorbed with C. albicans yeast cells (3153A) and DPBS. Control mice received 0.5 ml of the DPBS diluents or adsorbed mAb solution. For each condition, 6- to 8-week-old female BALB/c mice (NCI) were given 0.5 ml of test mAb, or control materials intraperitoneally, followed 4 h later by 0.1 ml intravenously of a suspension containing lethal dose of Candida cells per milliliter of DPBS. Mice were divided into groups with five mice each and three independent experiments were carried out. We have developed neutropenic murine models of disseminated infection by C. glabrata, C. tropicalis and C. parapsilosis. To induce neutropenia, naïve mice received a 200 mg/kg dose of cyclophosphamide (CY) by i.p. on day −3. Prolonged neutropenia were maintained for 30-50 days by giving each animal a 150 mg/kg dose of CY by i.p. every 10 days after infection. On day 0, BALB/c mice were given an i.p dose of each mAb four hours before being intravenously infected with 5×10⁵ viable C. albicans (3153A), 5×10⁶ C. parapsilosis ATCC MYA-4646 (A), 1×10⁸ C. glabrata ATCC 2001 cells (B) and 1×10⁷ C. tropicalis ATCC 28775 cells (C) in 0.1 ml DPBS.

TABLE 6
universal peptide-specific mAbs provide protection against
Non-albicans Candida spp in mouse model of disseminated

| | against C. | against C. | against C. | against C. |
|---|---|---|---|---|
| mAb | albicans | tropicalis | glabrata | parapsilosis |
| 2C9 | High (80-100%) | High (60-80%) | High (80-90%) | High (70-80%) |
| 5A9 | High (80-90%) | High (70-90%) | High (80-90%) | High (80-90%) |
| 5A1 | High (60-90%) | High (60-80%) | High (60-90%) | High (60-80%) |
| 6E3 | High (80-100%) | High (80-100%) | High (80-90%) | High (80-100%) |
| 1D5 | High (70-80%) | High (60-80%) | High (70-90%) | High (70-80%) |
| 7A3 | High (70-90%) | High (80-90%) | High (80-90%) | High (80-100%) |
| 10E7 | High (80-100%) | High (60-70%) | High (60-70%) | High (60-80%) |
| 1A11 | High (60-90%) | High (70-80%) | High (80-90%) | High (70-80%) |
| 10B6 | High (80-100%) | High (60-80%) | High (60-70%) | High (60-90%) |
| 5G5 | High (80-100%) | High (80-90%) | High (60-80%) | High (70-80%) |

Table 7 displays combinations of universal peptide-specific mAbs (also referred to as mAb cocktails) conferred enhanced protection against systemic candidiasis in passive transfer experiments as compared to single mAb treatment. Mice receiving single mAb treatment had significantly prolonged survival as compared to control animals that received either DPBS or adsorbed mAbs. However, the protective mAbs combination treatment provided the best protection (80-100% survival). Consistently, the group receiving treatment of multiple protective mAbs in combination had the least CFUs in kidneys among all the groups. To test the number of doses of each mAb needed for the complete protection in naïve mice, each mAb was further evaluated by giving to naïve mice every other day post challenge for two weeks. Interestingly, when mAb was given once, it provided 50-60% protection of recipient animals; however, when each mAb was given every other day for two weeks, it increased protection to 70-80% survival (Xin, H. “Double chimeric peptide vaccine and monoclonal antibodies that protect against disseminated candidiasis.” J Vaccines Vaccin 5 (2014)). However, the combination of protective mAbs provides the best protection with reduced dose of each mAb administered. Specifically, the combination mAb treatment provided 80-100% survival with one dose of mAbs in combination. In all the transfer experiments, passive protection was prevented by removal of the mAbs through absorption with C. albicans cells before transfer, which provided strong additional evidence for the protection being due to the mAbs.

TABLE 7

| protective mAb | Control | control | Protection of | Synergy of |
|---|---|---|---|---|
| cocktails | #1 | #2 | mAb cocktails | combinations |
| 2C9 + 1A11 | 2C9 | 1A11 | High (90-100%) | Yes++ |
| 2C9 + 10B6 | 2C9 | 10B6 | High (90-100%) | Yes++ |
| 2C9 + 5G5 | 2C9 | 5G5 | N/A | N/A |
| 2C9 + 10E7 | 2C9 | 10E7 | High (70-80%) | No |
| 5A9 + 10E7 | 5A9 | 10E7 | High (100%) | Yes++ |
| 5A9 + 1A11 | 5A9 | 1A11 | High (100%) | Yes++ |
| 5A9 + 10B6 | 5A9 | 10B6 | High (70-80%) | No |
| 5A9 + 5G5 | 5A9 | 5G5 | N/A | N/A |
| 6E3 + 10E7 | 6E3 | 10E7 | High (80-90%) | Yes |
| 6E3 + 10B6 | 6E3 | 10B6 | High (70-80%) | No |
| 6E3 + 1A11 | 6E3 | 1A11 | High (100%) | Yes+ + |
| 6E3 + 5G5 | 6E3 | 5G5 | N/A | N/A |
| 7A3 + 10E7 | 7A3 | 10E7 | High (70-80%) | No |
| 7A3 + 10B6 | 7A3 | 10B6 | High (90-100%) | Yes++ |
| 7A3 + 1A11 | 7A3 | 1A11 | High (90-100%) | Yes++ |
| 7A3 + 5G5 | 7A3 | 5G5 | High (90-100%) | Yes |

Table 8 displays that administration of combination of protective mAbs specific for peptides Fba, Met6 or Hwp1 conferred enhanced protection against systemic candidiasis in passive transfer as compared to single mAb treatment. For example, multiple protective mAbs specific for Fba, Met6 or Hwp1 peptide vaccines that were derived from different C. albicans cell wall proteins were combined into one cocktail and the combination cocktails were evaluated for protective efficacy in comparison with each protective mAb given alone. Survival data indicates that although the use of individual mAb can be a single prophylactic for infection, the combinations of multiple mAbs (detailed in Table 8) resulted in the best protective efficacies as evidenced by longer survival time, higher survival and significantly reduced/non-detectable CFU in kidneys when compared to group receiving single mAb treatment. This work demonstrates that the combination of monoclonal antibodies is a more efficient immune-protective approach against the disease as compared to single mAb treatment.

TABLE 8

| | | | | Protection |
|---|---|---|---|---|
| protective mAb | Control | control | Protection of | synergy of |
| cocktails | #1 | #2 | mAb cocktails | combination |
| 3H7 + 6H1 | 3H7 | 6H1 | High (90-100%) | Yes++ |
| 3H7 + 3B5 | 3H7 | 3B5 | High (90-100%) | Yes++ |
| 3H7 + 5B11 | 3H7 | 5B11 | N/A | N/A |
| 7H6 + 6H1 | 7H6 | 6H1 | N/A | N/A |
| 7H6 + 3B5 | 7H6 | 3B5 | High (100%) | Yes++ |
| 7H6 + 5B11 | 7H6 | 5B11 | High (100%) | Yes++ |
| 2D5 + 6H1 | 2D5 | 6H1 | N/A | N/A |
| 2D5 + 3B5 | 2D5 | 3B5 | N/A | N/A |
| 2D5 + 2B10 | 2D5 | 2B10 | High (80-90%) | Yes |
| 3H7 + 2D5 | 3H7 | 2D5 | N/A | N/A |
| 3H7 + 3E2 | 3H7 | 3E2 | High (80-100%) | Yes+ + |
| 3H7 + 6G9 | 3H7 | 6G9 | N/A | N/A |
| 3H7 + 9C11 | 3H7 | 9C11 | N/A | N/A |
| 3H7 + 7H5 | 3H7 | 7H5 | High (90-100%) | Yes++ |
| 7H6 + 2D5 | 7H6 | 2D5 | N/A | N/A |
| 7H6 + 3E2 | 7H6 | 3E2 | High (90-100%) | Yes |
| 7H6 + 6G9 | 7H6 | 6G9 | High (90-100%) | Yes++ |
| 7H6 + 9C11 | 7H6 | 9C11 | High (90-100%) | Yes++ |
| 7H6 + 7H5 | 7H6 | 7H5 | N/A | N/A |

Embodiments further comprise chimeric peptide constructs, such as double-peptide constructs, including those feasible for human vaccine immunization approach, and also those that do not require an adjuvant, in induction of protective immunity in mice.

### The Utility of Two Peptide Epitopes Combined in One Vaccine for Induction of a Greater Protective Immunity Against Disseminated Candidiasis.

Without wishing to be bound by theory, t double chimeric peptide vaccines can induce greater protection against disseminated candidiasis. For example, one antigenic universal peptide can be conjugated to the N terminus of a second peptide, such as a second antigenic universal peptide, through a linker, such as a double lysine linker (-KK-), to form a double chimeric peptide vaccine. Dendritic cell (DC) based immunization approach can be used to show protective efficacy (6). For example, five mice of each test group can be immunized with the double conjugate construct, the individual peptides themselves, or peptide mixture. Control groups can receive DPBS or DCs only. All the animals were challenged with lethal dose of live C. albicans 3153A cells by i.v.

Without wishing to be bound by theory, the double chimeric peptide vaccine, for example that which comprises two peptides expressed on the cell surface of C. albicans, can induce greater protective immunity than that induced by each individual peptide or by a simple mixture of both peptides. For example, the mice immunized with the double conjugate will have the least detectable CFUs in kidneys as compared to other immunized groups. The experiment can be terminated after 60 days, at which point CFUs in kidneys can be measured.

Improved Double Peptide Constructs Feasible for Human Use Induced Protection Against Disseminated Candidiasis in Mice without Need for Adjuvant.

Using two identified 14mer protective peptide vaccines as the unit peptide, a panel of double-peptide vaccines can be constructed by inserting various amino acid spacers (such as -KK- or GPSL (SEQ ID NO: 50) or RDG, or PADRE) between the two unit peptides, and adding T cell universal epitope (TT947-967 (SEQ ID NO: 78) or TT830-844 (SEQ ID NO: 75)) derived from tetanus toxoid at either N- or C-terminus of the peptide.

To demonstrate protective efficacy, groups of BALB/c mice can be immunized via the simple i.p. injections with each conjugate peptide vaccine, with or without any adjuvant. Following challenge with a lethal dose of C. albicans 3153A yeast cells, it is expected that the double-peptide constructs will induce 80-100% protection under adjuvant-free conditions in mice, leading to new peptide vaccine formulas with an immunization approach feasible for human use. It is expected that mice vaccinated with the double-peptide vaccines will survive significantly longer than control DPBS group as well as other immunized groups (p<0.001). It is anticipated that no CFUs or significantly reduced CFUs will be detected in kidneys of the mice immunized with the conjugate vaccines.

### Conclusions and Discussion for Improved Double-Peptide Vaccines.

Without wishing to be bound by theory, double peptide vaccine compositions comprising antigenic universal peptides provide solid protection against hematogenously disseminated candidiasis by C. albicans in both BALB/c and DAB/2 mice. A significant strength of the vaccine composition is that it contains multiple protective peptide epitopes inducing double protective immunity, feasible for simple i.p. immunization, and induces protection in the absence of an adjuvant. Furthermore, a neutropenic murine model of disseminated infection by three non-albicans (NAC) Candida spp has been established. Additional searches in the N-terminal region of full-length cell surface proteins of C. albicans have been performed, and identified multiple universal peptide candidates expressed at 100% homology by C. albicans, C. tropicalis, C. glabrata, C. parapsilosis, and C. Krusei (Table 2). Those peptides have the capability to induce strong antibody responses and protective immunity against disseminated candidiasis due to C. albicans. Therefore, without wishing to be bound by theory, the inclusion of universal peptides in the new double-peptide constructs provides broad coverage against these other medically significant Candida species.

### REFERENCES FROM THIS EXAMPLE, EACH OF WHICH ARE INCORPORATED BY REFERENCE HEREIN IN THEIR ENTIRETIES

1. Wilson, L. S., C. M. Reyes, M. Stolpman, J. Speckman, K. Allen, and J. Beney. 2002. The direct cost and incidence of systemic fungal infections. Value Health 5: 26-34.
2. Pappas, P. G., C. A. Kauffman, D. Andes, D. K. Benjamin, Jr., T. F. Calandra, J. E. Edwards, Jr., S. G. Filler, J. F. Fisher, B.-J. Kullberg, L. Ostrosky-Zeichner, A. C. Reboli, J. H. Rex, T. J. Walsh, and J. D. Sobel. 2009. Clinical practice guidelines for the management of candidiasis: 2009 update by the Infectious Diseases Society of America. Clin. Infect. Dis. 48: 503-535.
3. Pappas, P. G., J. H. Rex, J. Lee, R. J. Hamill, R. A. Larsen, W. Powderly, C. A. Kauffman, N. Hyslop, J. E. Mangino, S. Chapman, H. W. Horowitz, J. E. Edwards, W. E. Dismukes, and NIAID Mycoses Study Group. 2003. A prospective observational study of candidiemia: epidemiology, therapy, and influences on mortality in hosptilaized adult and pediatric patients. Clin. Infect. Dis. 37: 634-643.
4. Claudia, S., and D. Leonardi. 2013. Candida Infections, Causes, Targets, and Resistance Mechanisms: Traditional and Alternative Antifungal Agents. BioMed Research International 2013.
5. Spellberg, B. 2011. Vaccines for invasive fungal infections. F1000 Medicine Reports 3: 13.
6. Xin, H., S. Dziadek, D. R. Bundle, and J. E. Cutler. 2008. Synthetic glycopeptide vaccines combining b-mannan and peptide epitopes induce protection against candidiasis. Proc. Natl. Acad. Sci. USA 105: 13526-13531.
7. Xin, H., and J. E. Cutler. 2011. Vaccine and monoclonal antibody that enhance mouse resistance to candidiasis. Clin. Vaccine Immunol. 18: 1656-1667.
8. Xin, H., J. Cartmell, J. J. Bailey, S. Dziadek, D. R. Bundle, and J. E. Cutler. 2012. Self-adjuvanting glycopeptide conjugate vaccine against disseminated candidiasis. PLoS One 7.
9. Xin, H. 2014. Double Chimeric Peptide Vaccine and Monoclonal Antibodies That Protect Against Disseminated Candidiasis. J Vaccines Vaccin 5.
10. Xin, H. 16 A.D. Active immunizations with peptide-DC vaccines and passive transfer with antibodies protect neutropenic mice against disseminated candidiasis. Vaccine 34: 245-251.
11. Filler, S. G., and J. E. Edwards. 1995. When and how to treat serious candidial infections: Concepts and controversies. Curr. Clin. Top. Infect. Dis. 15: 1-18.
12. Chung, J. W., S. O. Lee, S. H. Choi, J. H. Woo, J. Ryu, Y. S. Kim, and N. J. Kim. 2006. Risk factors and outcome for breakthrough candidemia in patients with cancer. Mycoses 49: 114-118.
13. Saylor, C., E. Dadachova, and A. Casadeval. 2009. Monoclonal antibody-based therapies for microbial diseases. Vaccine 27 (Suppl 6): 38-46.
14. Han, Y., and J. E. Cutler. 1995. Antibody response that protects against disseminated candidiasis. Infect. Immun. 63: 2714-2719.
15. Han, Y., M. H. Riesselman, and J. E. Cutler. 2000. Protection against candidiasis by an immunoglobulin G3 (IgG3) monoclonal antibody specific for the same mannotriose as an IgM protective antibody. Infect. Immun. 68: 1649-1654.
16. Son, Y.-I., S. Egawa, T. Tatsumi, R. E. Redlinger, P. Kalinski, and T. Kanto. 2002. A novel bulk-culture method for generating mature dendritic cells from mouse bone marrow cells. J. Immunol. Meth. 262: 145-157.
17. Xin, H., B. L. Granger, S. Dziadek, D. R. Bundle, and J. E. Cutler. 2007. Antigen-pulsed dendritic cells used for identification of carrier peptides and eventual glycopeptide conjugate vaccines against candidiasis. ASM Proc. (Abst).
18. Guinea, J. 2014. Global trends in the distribution of Candida species causing candidemia. Clin. Microbiol. Infect. 20: 5-10.
19. Arendrup, M., T. Horn, and N. Frimodt-Moller. 2002. In vivo pathogenicity of eight medically relevant Candida species in an animal model. Infection 30: 286-291.
20. MacCallum, D. M. 2012. Hosting Infection: Experimental Models to Assay Candida Virulence. International Journal of Microbiology 2012: 1-12.

### Example 4

Vaccination with universal peptide vaccines in mice to demonstrate induced protective antibodies and protection against all the medically important Candida spp. Novel universal peptides as described herein can be excellent peptide vaccines against C. albicans infection. The universal peptide vaccines have been tested against other important opportunistic Candida species, including C. tropicalis, C. glabrata and C. parapsilosis in addition to C. albicans to demonstrate that the range of protection by a peptide vaccine against disseminated candidiasis can be broadened by incorporating a cell surface peptide generally expressed by medically important Candida spp.

By the established powerful DC-based immunization strategy favoring production of antibodies, a subset of the universal Candida surface peptide candidates (for example, GPV-M1, GPV-M2, GPV-P1, GPV-P3) was able to provide broad coverage against a variety of medically important Candida spp, which cause >85% disseminated candidiasis in humans.

Further, monoclonal antibodies specific to the universal peptides have been produced and/or isolated which demonstrate solid protection in mouse model of disseminated candidiasis caused by C. albicans, C. tropicalis and C. glabrata (Table 9).

TABLE 9
Universal peptide vaccines protect against disseminated
candidiasis caused by C. tropicalis and C. glabrata
in addition to C. albicans:

| | | | | Protection against Candida | | |
|---|---|---|---|---|---|---|
| Universal Peptide Vaccines | | | | spp in mouse model | | |
| and Related mAbs | | | | Against | Against | Against |
| Hybrid- | mAb | Peptide | Peptide | C. | C. | C. |
| omas | isotype | vaccine | Sequences | albicans | tropicalis | glabrata |
| 2C9G3 | IgG2a | GPV-M2 | cTTTIGSFPQTKDIR | High | High | High |
| | | universal | (SEQ ID NO: 2) | (80-100%) | (60-80%) | (80-90%) |
| 5A9D1 | IgG3 | GPV-M2 | cTTTIGSFPQTKDIR | High | High | not yet |
| | | universal | (SEQ ID NO: 2) | (80-90%) | (70-90%) | tested |
| 5A11B1 | IgG3 | GPV-M2 | cTTTIGSFPQTKDIR | High | High | High |
| | | universal | (SEQ ID NO: 2) | (60-90%) | (60-80%) | (60-90% |
| 6E3D9 | IgG1 | GPV-M1 | cFWVNPDCGLKTR | High | High | not yet |
| | | universal | (SEQ ID NO: 1) | (80-100%) | (80-100% | test |
| 1D4H5 | IgG1 | GPV-M1 | cFWVNPDCGLKTR | High | High | High |
| | | universal | (SEQ ID NO: 1) | (70-80%) | (60-80%) | (70-90%) |
| 7A3C4 | IgG1 | GPV-M1 | cFWVNPDCGLKTR | High | High | High |
| | | universal | (SEQ ID NO: 1) | (70-90%) | (80-90%) | (80-90%) |
| 10E7E2 | IgG1 | GPV-P3- | cIVIIGGGDTATVAKK | High | High | High |
| | | universal | (SEQ ID NO: 16) | (80-100% | (60-70%) | (60-70%) |
| 1A11H8 | IgG3 | GPV-P1 | cAILGGSKVSDKI | High | High | not yet |
| | | universal | (SEQ ID NO: 14) | (60-90%) | (70-80%) | tested |
| 10B6H8 | IgG1 | GPV-P1 | cAILGGSKVSDKI | High | High | High |
| | | universal | (SEQ ID NO: 14) | (80-100% | (60-80%) | (60-70%) |
| 5G5G3 | IgG2a | GPV-P1 | cAILGGSKVSDKI | High | High | High |
| | | universal | (SEQ ID NO: 14) | (80-100% | (80-90%) | (60-80%) |

As described herein are examples of universal peptide vaccines that can elicit antibody responses and high degree protection against disseminated candidiasis due to Candida spp., such as those described above.

New Universal Peptide Candidates Met6-2, FbaU1 Induced High Degree Protection Against Disseminated Candidiasis Caused by C. albicans in Mice

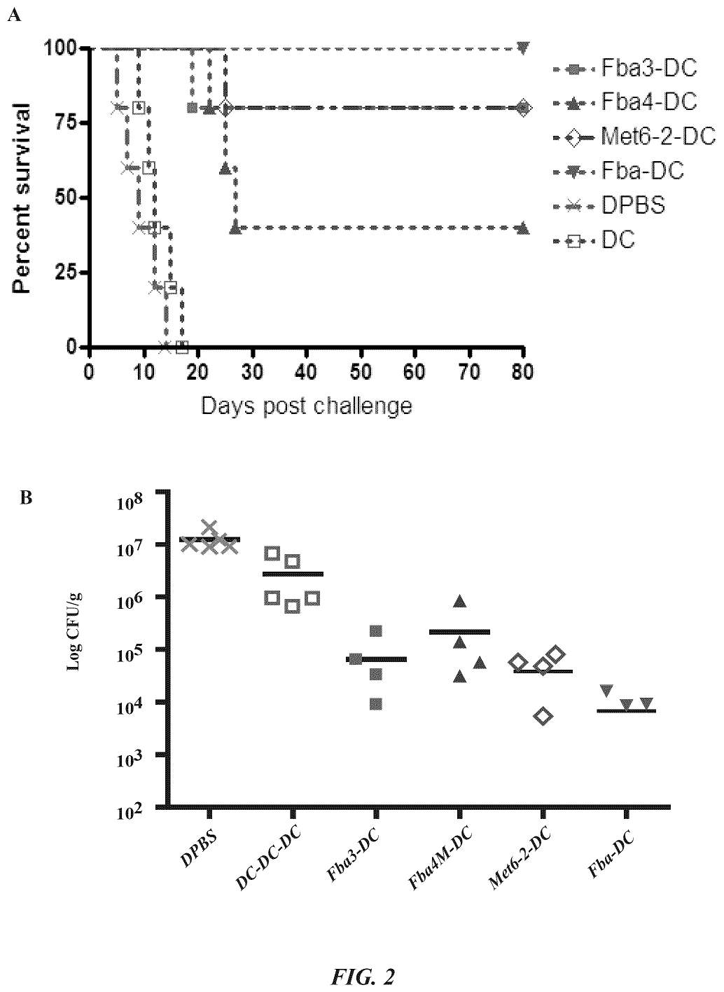

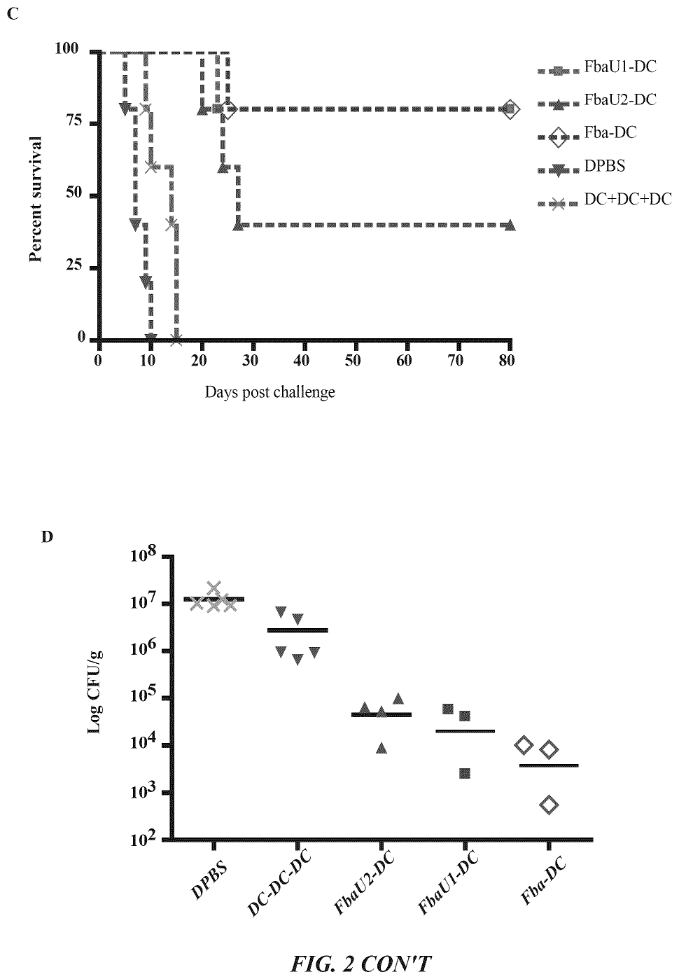

To perform rapid throughput evaluation of peptide vaccine candidates, immunizations were done by use of the similar antigen-pulsed DC based vaccine strategy with modification by totally removing CFA from vaccine formula. Fba peptide was used as positive control. All five putative universal peptides [Fba3 (also referred to as FbaU1; SEQ ID NO: 36; FAIPAINVTSSSTVVAALE); Fba4 (SEQ ID NO. 37; SSSTVVAAL); Met6-2 (SEQ ID NO. 38; YDQVLDLSLLFNAIP); FbaU1; and FbaU2 (SEQ ID NO. 80; PAINVTSSSTVVAALEAA)] were able to induce specific antibody responses in BALB/c mice (data not shown), and importantly, peptides Met6-2, Fba3 and FbaU1 induced good protective responses against disseminated candidiasis caused by C. albicans (FIG. 2); accordingly, the protected groups had reduced or non-detectable CFUs in their kidneys as compared to controls (FIGS. 2, B and D). Passive transfer of immune sera showed the antibodies are responsible for the protection.

The Range of Protection by Universal Peptide Vaccines can be Broadened to Protect Against Disseminated Candidiasis Caused by C. tropicalis

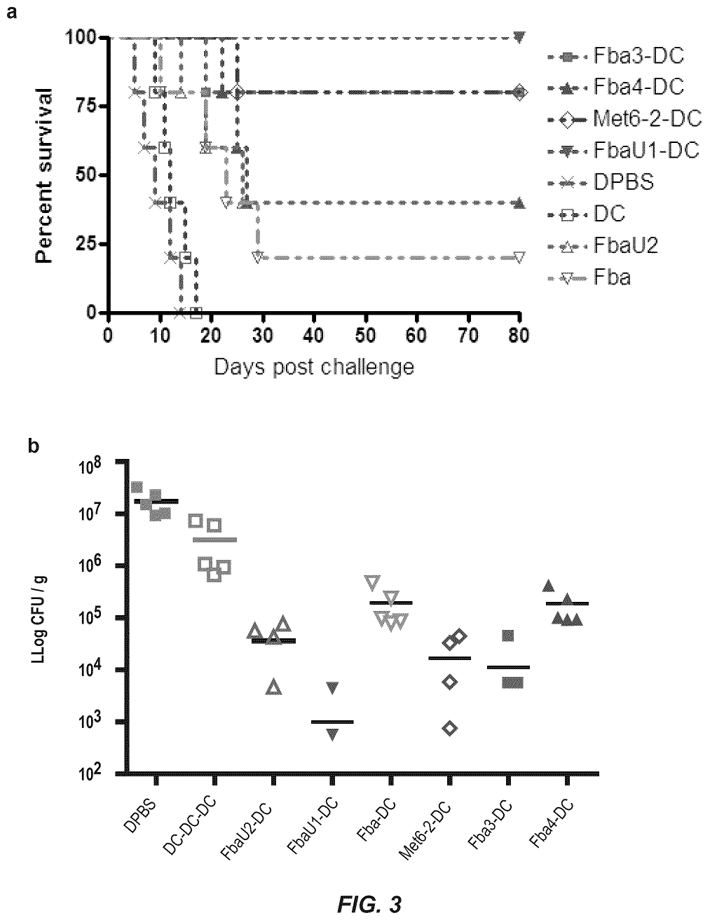

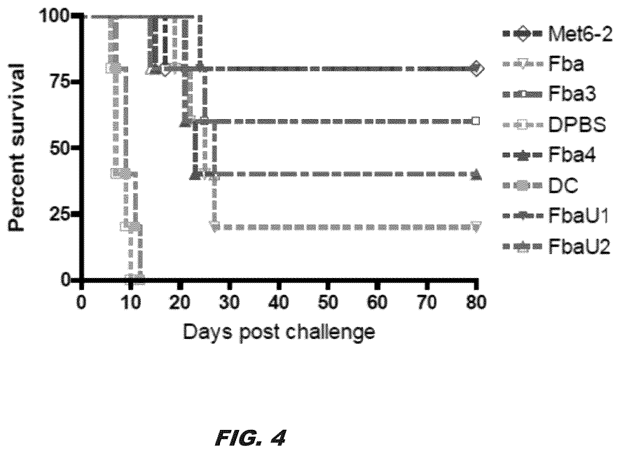

Peptide-vaccinated mice were challenged with lethal dose of live C. tropicalis cells (ATCC 20336; i.v. challenge dose: 1×106/mouse). Peptide FbaU1 induced the best protection (100% survival) among the peptide candidates; both Fba3 and Met6-2 peptides induced good protection with 80% survival up to 80 days (FIG. 3A). CFU data in kidneys of immunized groups are very consistent with the survival data (FIG. 3B), FbaU1, Fba3 and Met6-2 groups had significantly reduced live fungal cells in their kidneys as compared to controls (p<0.001).

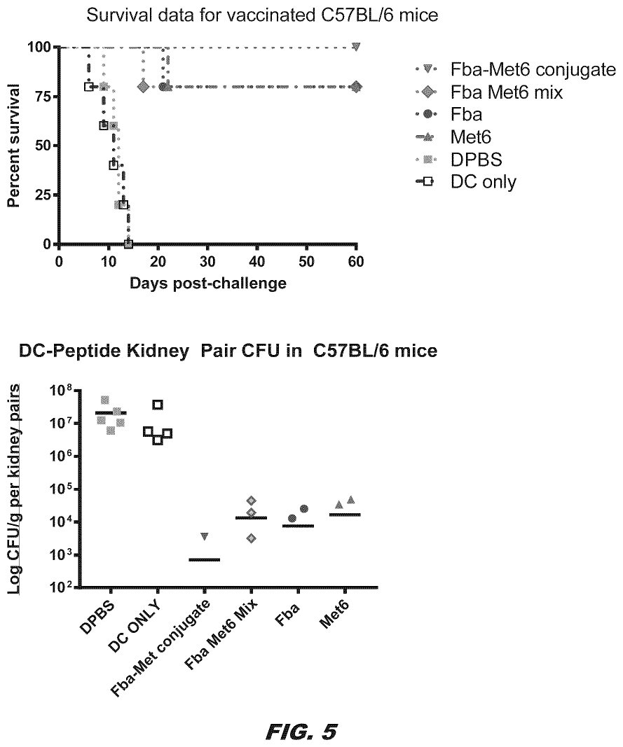

The range of protection by universal peptides can be broadened to protect against disseminated candidiasis caused by C. glabrata Peptide vaccinated mice were also challenged with C. glabrata, which has emerged as the second most common cause of invasive candidiasis in the US. Since C. glabrata is not lethal for wild-type mice, immunosuppression in vaccinated mice were induced by cyclophosphamide (50 mg/kg) treatment for two weeks, and then mice were challenged with live C. glabrata cells [CBS138 (ATCC 2001), 1×107 cells/mouse]. Peptide FbaU1& Met6-2 peptides were able to induce a high degree protection (FIG. 4). CFU experiments will be performed and completed. Without wishing to be bound by theory, survival data is correlated with the number of CFUs in kidneys of experimental mice. Expected results comprise a significant reduction in number of CFUs in kidneys of mice vaccinated with peptide FbaU1 or Met6-2 peptide vaccine as compared to controls.

Vaccination with Universal Double Chimeric or Multiple Peptide Vaccines in Mice to Show Enhanced Efficacy and Synergistic Protection Against Disseminated Candidiasis.

TABLE 10
New universal double/multiple peptide vaccines against disseminated candidiasis:

| SEQ | | Double Peptide Vaccine Formula | |
|---|---|---|---|
| ID NO | | Feasible for Human Use | Modification |
| 83 | 1 | PADRE-GPV-M1-GPSL-GPV-P1 | Amidation (C-terminus) and |
| | | | Acetylation (N-terminus) |
| 84 | 2 | TT830-844-GPSL-GPV-M1-GPSL-GPV-P1 | Amidation (C-terminus) and |
| | | | Acetylation (N-terminus) |
| 85 | 3 | TT947-967-GPSL-GPV-M1-GPSL-GPV-P1 | Amidation (C-terminus) and |
| | | | Acetylation (N-terminus) |
| 86 | 4 | RGD-GPV-P3-KK-GPV-M2 | Amidation (C-terminus) and |
| | | | Acetylation (N-terminus) |
| 87 | 5 | RGD-GPV-P3-KK-GPV-M2-KLH | Amidation (C-terminus) and |
| | | | Acetylation (N-terminus) |
| | | | KLH Conjugation on Cysteine |
| 88 | 6 | TT830-844-KK-GPV-P3-KK-GPV-M2 | Amidation (C-terminus) and |
| | | | Acetylation (N-terminus) |
| 89 | 7 | TT947-967-KK-GPV-P3-KK-GPV-M1 | Amidation (C-terminus) and |
| | | | Acetylation (N-terminus) |
| 90 | 8 | RGD-TT830-844-KK-GPV-P3-KK-GPV-M1 | Amidation (C-terminus) and |
| | | | Acetylation (N-terminus) |
| 91 | 9 | RGD-TT947-967-KK-GPV-P3-KK-GPV-M1 | Amidation (C-terminus) and |
| | | | Acetylation (N-terminus) |
| 92 | 10 | PADRE-GPV-M2-KK-GPV-P1 | Amidation (C-terminus) and |
| | | | Acetylation (N-terminus) |
| 93 | 11 | RGD-PADRE=GPV-M2-KK-GPV-P3 | Amidation (C-terminus) and |
| | | | Acetylation (N-terminus) |

As described herein, universal double/multiple peptide vaccines against disseminated candidiasis have been developed. For example, 11 double chimeric peptide vaccines, each with different linkers, have been developed, along with vaccines formulated for human use (Table 10). Each chimeric peptide conjugate comprises two peptide epitopes.

Further, compositions and methods of animal immunizations feasible for human use, for example vaccination without any need for adjuvant, have been developed.

The double chimeric peptide conjugate vaccines have been tested for their abilities to induce specific anti-peptide antibodies and protective immunity against the disease caused by C. albicans. In this example, universal double-peptide vaccines were able to induce robust antibody responses, as well as provided 60-100% survival up to 50 days in immunized mice, which showed enhanced protection as compared to each individual universal peptide vaccine. The enhancement in vaccine efficacy was evidenced by both increased survival and reduced or non-detectable fungal burden (colony forming units, CFUs) in kidneys of animals.

As described herein are double chimeric universal peptide conjugate vaccines composed of peptides universally produced by medically important Candida species in addition to C. albicans. An enhanced immune response offering protection against disseminated candidiasis due to C. albicans was also detected in a mouse model of human disseminated candidiasis.

A subset of the universal double chimeric peptide vaccines detected will be tested to show their protective efficacy against other clinically Candida important species.

TABLE 11
universal double-peptide vaccines induce solid and synergistic
protection.

| | Protection against | |
|---|---|---|
| Double Peptide vaccine formula | Candidiasis in mouse model | |
| feasible for human use | | Synergy |
| 1. PADRE-GPV-M1-GPSL-GPV-P1 | High (60-90%) | + + |
| 2. TT830-844-GPSL-GPV-M1-GPSL-GPV-P1 | medium (40-60%) | |
| 3. TT947-967-GPSL-GPV-M1-GPSL-GPV-P1 | medium (40-60%) | |
| 4. RGD-GPV-P3-KK-GPV-M2 | High (70-90%) | + + |
| 5. RGD-GPV-P3-KK-GPV-M2-KLH | High (80-100%) | + + |
| 6. TT830-844-KK-GPV-P3-KK-GPV-M2 | medium (40-60%) | |
| 7. TT947-967-KK-GPV-P3-KK-GPV-M1 | High (70-90%) | |
| 8. RGD-TT830-844-KK-GPV-P3-KK-GPV-M1 | High (80-100%) | + + |
| 9. RGD-TT947-967-KK-GPV-P3-KK-GPV-M1 | Low (20-40%) | |
| 10. PADRE-GPV-M2-KK-GPV-P1 | Low (20-40%) | |
| 11. RGD-PADRE-GPV-M2-KK-GPV-P3 | High (80-100%) | + + |

### Combined Universal-Peptides-Related MAbs Will be Tested for Synergistic Protection Against Disseminated Candidiasis.

Combined universal-peptides-related MAbs have been tested for synergistic protection against disseminated candidiasis to demonstrate combinations of multiple protective MAbs specific to different universal peptides provide an effective form of passive transfer protection.

As described herein the combination of MAbs is a more efficient immunoprotective approach against candidiasis as compared to the single MAb treatment. In this study, the protective efficacy of universal-peptide-related MAbs in combination against disseminated candidiasis was further evaluated. This is important, the use of peptide vaccines, for example, in immunosuppressed patients can potentially be limited, as these patients may not necessarily mount active protective responses.

As described herein, the combination of MAbs is a more efficient immunoprotective approach against candidiasis as compared to the single MAb treatment (Table 12).

Several monoclonal antibodies specific for the defined universal peptide vaccines have been obtained by use of standard hybridoma techniques. Development of peptide-specific MAbs provided an unlimited supply of protective antibodies for in vivo applications. Initial results demonstrate that MAbs combination (M2-4/E2-9) treatment provides the best protection (10000 survival) as compared to single MAb treatment. Such results have been extended to include specific for novel universal peptides.

TABLE 12
Combinations of multiple protective MAbs that specific
to different universal peptides may provide the most
effective form of passive transfer protection.

| | | | Protection | |
|---|---|---|---|---|
| Therapeutic mAb | Control | control | of mAb | Synergy of |
| cocktails | #1 | #2 | cocktails | combination |
| 2C9G3 + 10E7E2 | 2C9G3 | 10E7E2 | High | Yes+ + + |
| | | | (80-100%) | |
| 5A9D1 + 10E7E2 | 5A9D1 | 10E7E2 | High | Yes+ + |
| | | | (80-90%) | |
| 1D4H5 + 1A11H8 | 1D4H5 | 1A11H8 | High | No |
| | | | (80-90%) | |
| 6E3D9 + 10B6H8 | 6E3D9 | 10B6H8 | High | No |
| | | | (80-100%) | |
| 7A3C4 + 5G5G3 | 7A3C4 | 5G5G3 | High | Yes+ + |
| | | | (70-90%) | |
| 5A11B1 + 10E7E2 | 5A11B1 | 10E7E2 | High | Yes+ + |
| | | | (90-100%) | |

### Example 5

Aspects of the invention described herein provide a panel of protective monoclonal antibodies (mAbs) targeting cell surface antigens of medically important Candida spp cell surface antigens. For example, clone 9F2, a IgG2a mAb, which is specific for a 14-mer peptide Pgk1 (VPLDGKTITNNQRI (SEQ ID NO: 34)) derived from Candida cell wall protein Phosphoglycerate kinase 1, protected against invasive candidiasis in mouse models. Peptide PgK1 is a surface epitope with high homology in medically important Candida species, including C. albicans, C. glabrata, C. tropicalis and C. auris.

As shown in an in vitro assay, for example, 9F2 mAb IgG2a is protective on its own in Candida inoculated culture, without need of any immune cells, antifungal agents or effector cells. Further, fungicidal and growth inhibition assays show 9F2 mAb, for example, can inhibit proliferation of C. auris yeast cells. At concentration of 9F2 mAb (200 μg/mL), no colony was detected and viability of C auris was zero. Therefore, 9F2 can function as both direct candidacidal killer to eliminate the live C. auris yeast cells and inhibit fungal growth.

Using our established in vivo mouse model of disseminated infection that closely mimics human candidiasis, we showed that two of our peptide-specific mAbs, clone 9F2, which target phosphoglycerate kinase 1 (Pgk1) and clone 6H1, which targets a 14-mer peptide derived from hyphal wall protein 1 (Hwp1), provided solid protection against invasive candidiasis, as evidenced by higher survival and lower fungal burdens in target organs compared to control mice. Furthermore, we showed that passive immunization using a 6H1+9F2 cocktail induced significantly enhanced protection against C. auris compared to treatment with either mAb individually. Our data demonstrate the utility of peptide-related mAbs as an effective alternative to antifungals against medical important Candida species including multidrug-resistant C. auris.

Without wishing to be bound by theory, fungicidal mAbs with inhibitory activity against the growth of the fungal pathogen C. albicans are a ready-to-use antimicrobial immunotherapeutic for topical application and other therapeutic approaches.

Without wishing to be bound by theory, a therapeutic application of the mAbs includes an application in combination with current antifungals. Overall, the results of the present study indicating that mAb interference with growth and direct killing represents a useful strategy for the control of Candida infection under conditions of phagocyte ablation or functional deficiency. Killer or growth inhibiting antibodies appear to fulfill this requirement with high therapeutic efficacy and virtually no toxicity. Finally, lowering the local fungal burden, as shown for clone 9F2, for example, will contribute to the determination of the appropriate antifungal response through reduction of inflammatory pathology and fungal sepsis.

MAb as an adjunct to existing therapy: Despite the availability of a variety of antifungal drugs, opportunistic fungal infections still remain life-threatening for immunocompromised patients, such as those undergoing allogeneic hematopoietic cell transplantation or solid organ transplantation. Suboptimal efficacy, toxicity, development of resistant variants and recurrent episodes are limitations associated with current antifungal drug therapy. Antifungal drugs often fail to eradicate the infection, mainly in immunocompromised individuals, and fungicidal mAb, for example mAb 9F2 IgG2a, can enhance the impaired host defense and be used as an adjunctive immune therapy for invasive fungal diseases.

### Example 6

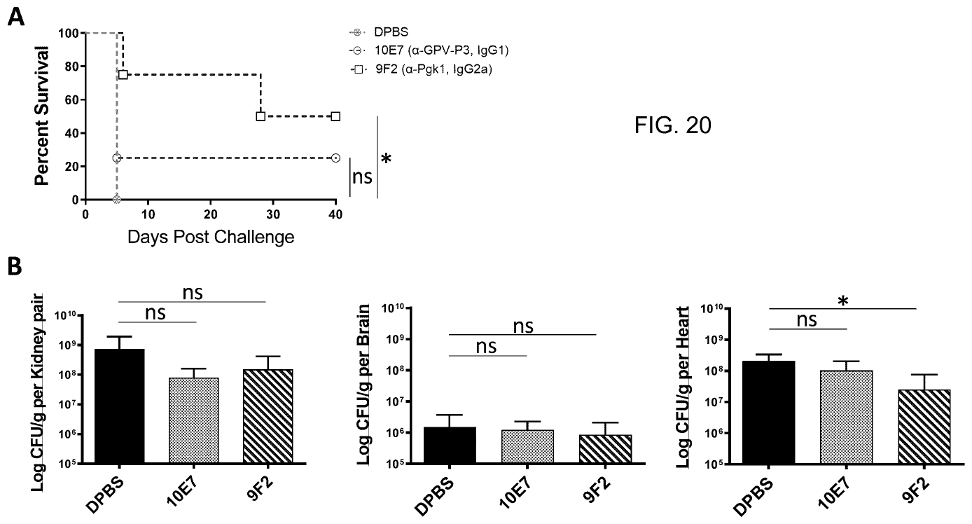

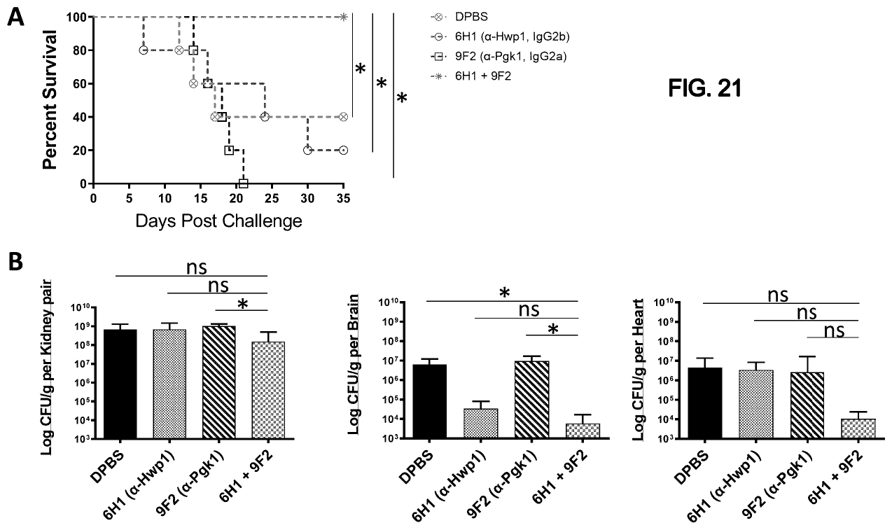

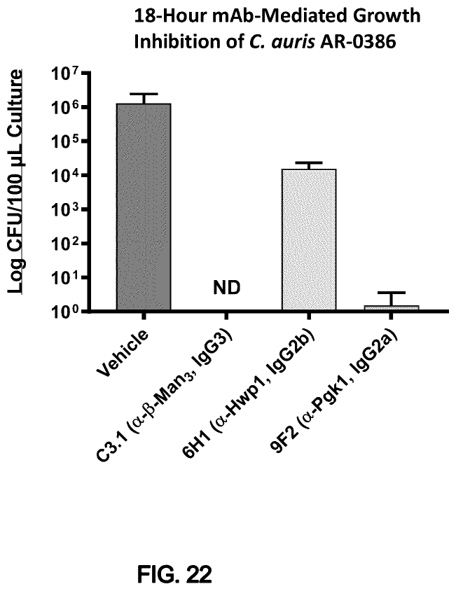

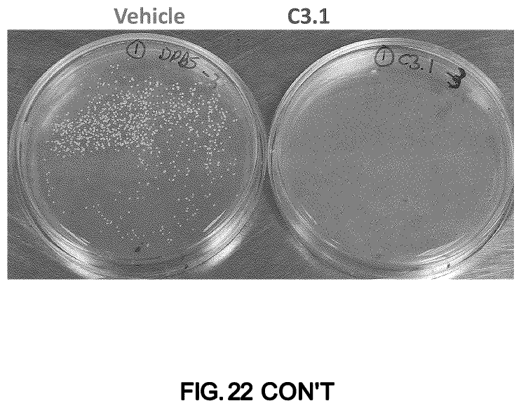

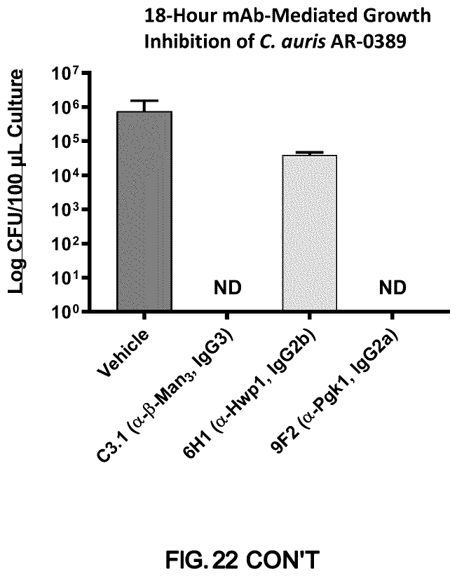

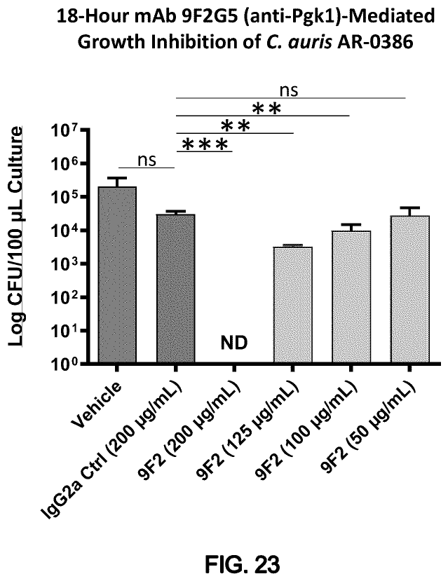

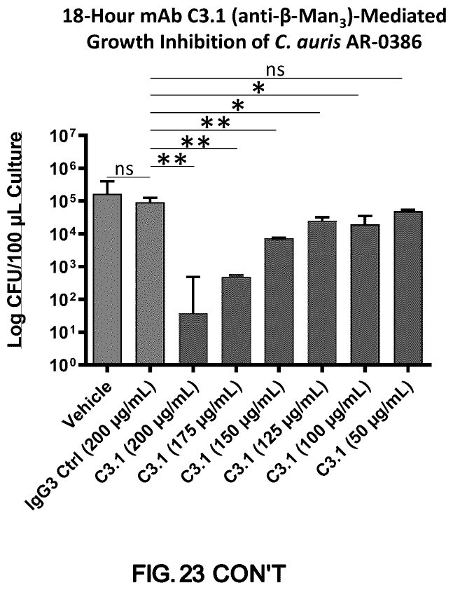

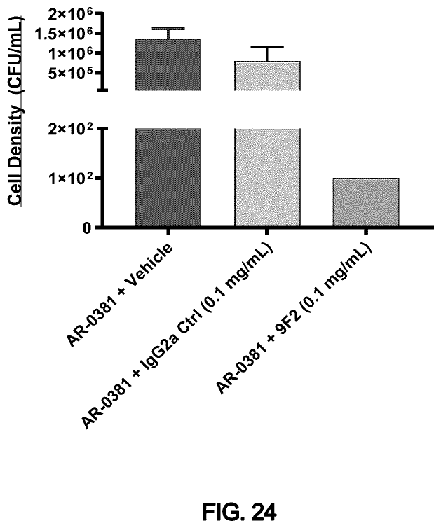

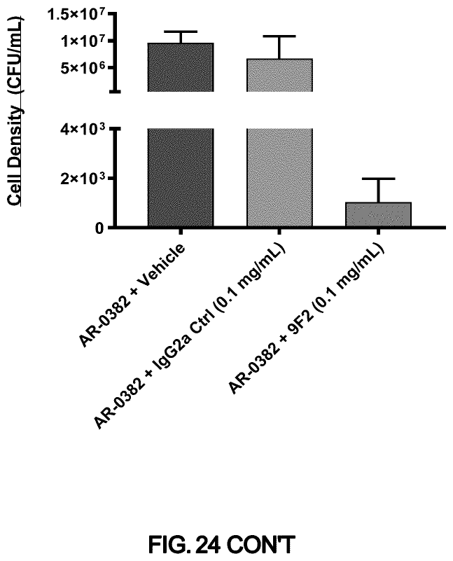

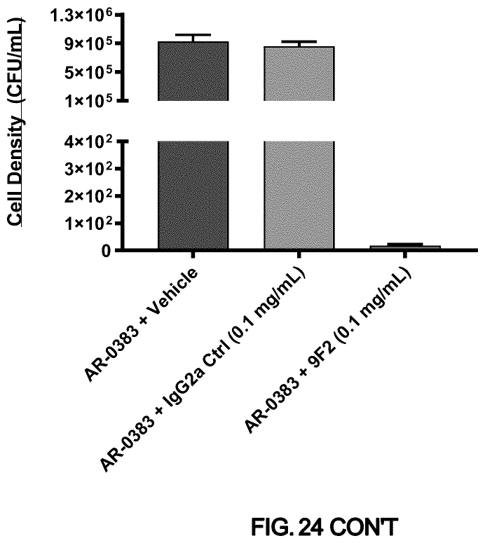

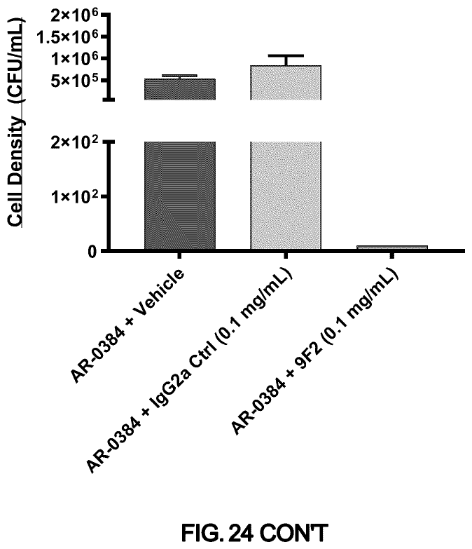

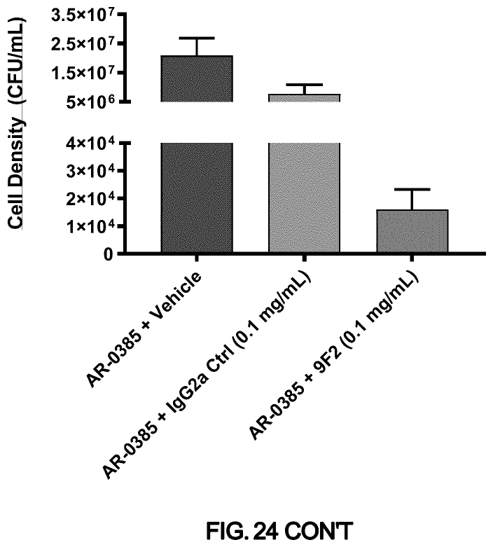

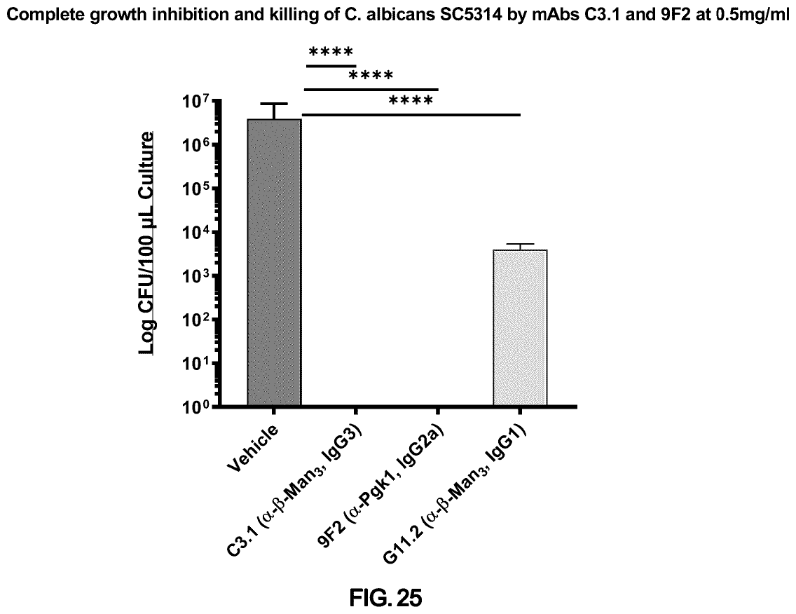

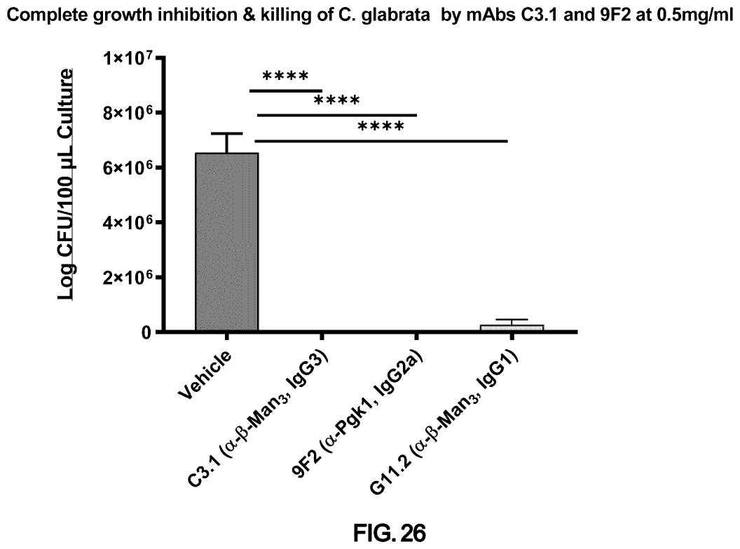

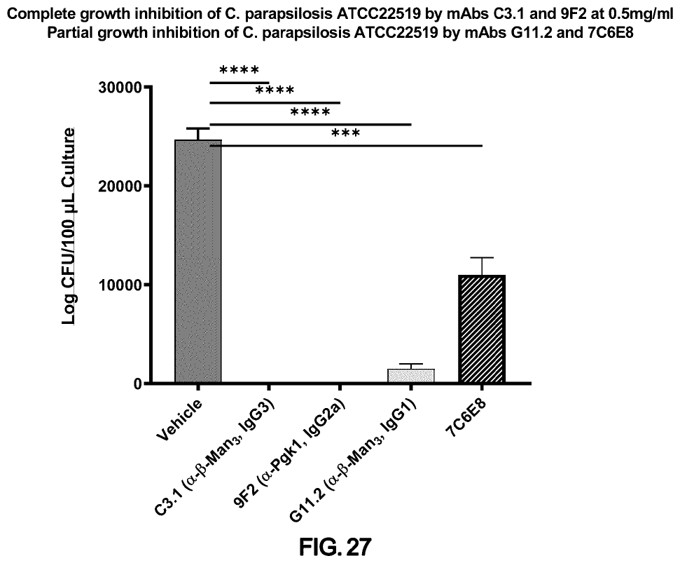

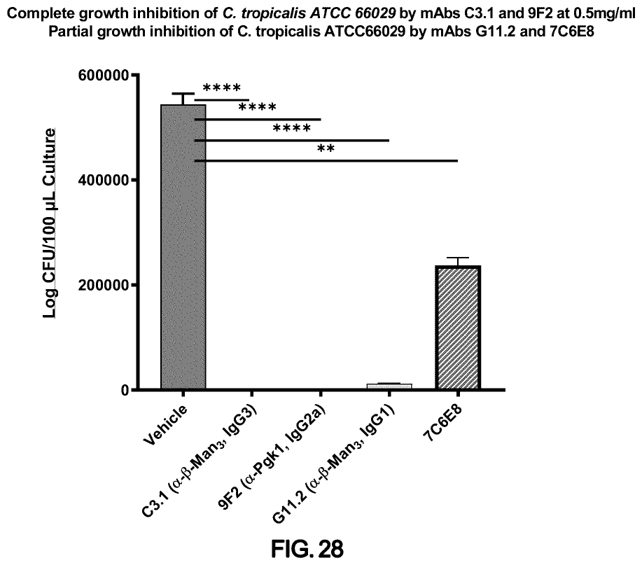

Overall conclusions from experiments as described in FIG. 20-28, for example.
1. Universal mAb Binding: Universal mAbs, for example, C3.1 (anti-β-Man3) and 9F2 (anti-Pgk1) show isolate-specific binding to two C. auris clinical isolates and to C. albicans.
2. Prophylactic Efficacy: 9F2 and C3.1 mAbs, for example, provide significantly extended survival and reduced fungal burdens in C. auris-infected mice compared to control mice.
3. C3.1 Timepoint Study:
   A. C3.1 mAb treatment enhances fungal clearance within the first 7 days of disseminated C. auris infection
   B. C3.1 treatment reduces the levels of pro-inflammatory cytokines in mice.
4. MAb Cocktails: Individually protective mAbs, for example 6H1 (anti-Hwp1) and 9F2, provide enhanced protection against disseminated C. auris infection when administered as a cocktail.
5. Phagocytosis: 9F2 and C3.1 mAbs, for example, enhance phagocytosis of C. auris yeast cells by RAW macrophages.
6. Growth Inhibition: 9F2 and C3.1 mAbs, for example, inhibit proliferation of C. auris yeast cells.

### Example 7

### Leader Sequence-FR1-CDR1-FR2-CDR2-FR3-CDR3-FR4

### 2B10C1 IgG1 mAb Sequencing Results and Analysis

Five single colonies with correct V_{H} and V_{L} insert sizes were sent for sequencing. The V_{H} and V_{L} genes of five different clones were found nearly identical. The consensus sequence, listed in Table 38, is the sequence of the antibody produced by the hybridoma 2B10C1.

TABLE 38
Heavy chain variable region: DNA sequence (405 bp) (SEQ ID NO: 59)
ATGGGATGGAGCTATATCATCCTCTTCTTGTTAGCAACAGCTACACGTGTCCACTCCCAGG
TCCAACTGCAGCAGCCTGGGGCTGAGGTGGTGAGGCCTGGGGCTTCAGTGAAGGTGTCCTG
CAAGGCTTCTGGCTACACGGTCAGCAGCTACTGGATGAGCTGGGTTAAGCAGAGGCCGGAG
CAAGGCCTTGAGTGGATTGGAAGGATTGATCCTTACGATAGTGAAACTCACTACAATCAAA
AGTTCAAGGACAAGGCCATATTGACTGTAGACAAATCCTCCAGCACAGCCTACATGCAACT
CAGCAGCCTGACATCTGAGGACTCTGCGGTCTATTACTGTGCAAGGACGGCCGCTTCGTTT

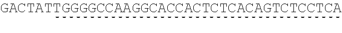

Heavy chain variable region: Amino acid sequence (135 AA) (SEQ ID NO: 60)
MGWSYIILFLLATATRVHSQVQLQQPGAEVVRPGASVKVSCKASGYTVSSYWMSWVKQRPE
QGLEWIGRIDPYDSETHYNQKFKDKAILTVDKSSSTAYMQLSSLTSEDSAVYYCARTAASF

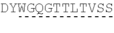

Light chain variable region: DNA sequence (393 bp) (SEQ ID NO: 61)
ATGAAGTTGCCTGTTAGGCTGTTGGTGCTGATGTTCTGGATTCCTGCTTCCAGCAGTGATG
TTGTGATGACCCAAACTCCACTCTCCCTGCCTGTCAGTCTTGGAGATCAAGCCTCCATCTC
TTGCAGATCTAGTCAGAGCCTTGTACACAGTAATGGAAACTCCTATTTACATTGGTACCTG
CAGAAGCCAGGCCAGTCTCCAAAGCTCCTGATCTACAAAGTTTCCAACCGATTTTCTGGGG
TCCCAGACAGGTTCAGTGGCAGTGGATCAGGGACAGATTTCACACTCAATATCAGCAGAGT

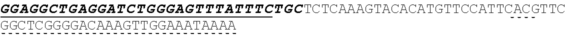

Light chain variable region: Amino acid sequence (131 AA) (SEQ ID NO: 62)
MKLPVRLLVLMFWIPASSSDVVMTQTPLSLPVSLGDQASISCRSSQSLVHSNGNSYLHWYL

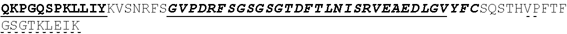

### 1D4H5 IgG1 GPV-M1 mAb Sequencing Results and Analysis

Five single colonies with correct V_{H} and V_{L} insert sizes were sent for sequencing. The V_{H} and V_{L} genes of five different clones were found nearly identical. The consensus sequence, listed in Table 39, is the sequence of the antibody produced by the hybridoma 1D4H5.

TABLE 39
Heavy chain variable region: DNA sequence (399 bp) (SEQ ID NO: 63)
ATGTACTTGGGACTGAACTGTGTATTCATAGTTTTTCTCTTAAAAGGTGTCCAGAGTGAAG
TGAAGCTTGAGGAGTCTGGAGGAGGCTTGGTGCAACCTGGAGGATCCATGAAACTCTCCTG
TGTTGCCTCTGGATTCACTTTCAGTAACTACTGGATGAACTGGGTCCGCCAGTCTCCAGAG
AAGGGGCTTGAGTGGGTTGCTGAAATTAGATTGAAATCTAATAATTATGCAACACATTATG
CGGAGTCTGTGAAAGGGAGGTTCACCATCTCAAGCGATGATTCCAAAAGTAGTGTCTACCT
GCAAATGAACAACTTAAGAGCTGAAGACACTGGCATTTATTACTGTTCAACTGGGAACTAC

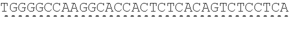

Heavy chain variable region: Amino acid sequence (133 AA) (SEQ ID NO: 64)
MYLGLNCVFIVFLLKGVQSEVKLEESGGGLVQPGGSMKLSCVASGFTFSNYWMNWVRQSPE
KGLEWVAEIRLKSNNYATHYAESVKGRFTISSDDSKSSVYLQMNNLRAEDTGIYYCSTGNY

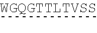

Light chain variable region: DNA sequence (390 bp) (SEQ ID NO: 65)
ATGGATTTTCAGATGCAGATTATCAGCTTGCTGCTAATCAGTGTCACAGTCATAGTGTCTA
ATGGAGAAATTGTGCTCACCCAGTCTCCAACCACCATGGCTGCATCTCCCGGGGAGAAGAT
CACTATCACCTGCAGTGCCAGCTCAACTATAAGTTCCAATTACTTGCATTGGTATCAGCAG
AAGCCAGGATTCTCCCCTAAACTCTTGATTTATAGGACATCCAATCTGGCTTCTGGAGTCC
CAGCTCGCTTCAGTGGCAGTGGGTCTGGGACCTCTTACTCTCTCACAATTGGCACCATGGA

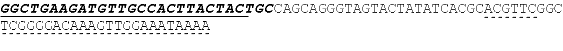

Light chain variable region: Amino acids sequence (130 AA) (SEQ ID NO: 66)
MDFQMQIISLLLISVTVIVSNGEIVLTQSPTTMAASPGEKITITCSASSTISSNYLHWYQQ

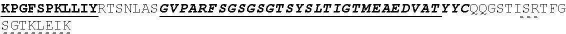

### 2D5F7IgG2b mAb Sequencing Results and Analysis

Five single colonies with correct V_{H} and V_{L} insert sizes were sent for sequencing. The V_{H} and V_{L} genes of five different clones were found nearly identical. The consensus sequence, listed in Table 40, is the sequence of the antibody produced by the hybridoma 2D5F7.

TABLE 40
Heavy chain variable region: DNA sequence (420 bp) (SEQ ID NO: 67)
ATGGAAAGGCACTGGATCTTTCTCTTCCTGTTGTCAGTAACTGCAGGTGTCCACTCCCAGG
TCCAGCTGCAGCAGTCTGCAGCTGAACTGGCAAGACCTGGGGCCTCAGTGAAGATGTCCTG
CAAGGCTTCTGGCTACACCTTTAGTAGCTACACGATGCACTGGGTAAAACAGAGGCCTGGA
CAGGGTCTGGAATGGATTGGATACATTAATCCTAGCAGTGGATATACTGATTACAATCAGA
AGTTCAAGGACAAGACCACATTGACTGCAGACAAATCCTCCAGCACAGCCTACATGCAACT
GAGCAGCCTGACATCTGAGGACTCTGCGGTCTATTACTGTGCAAGACTATATGATAACTAC

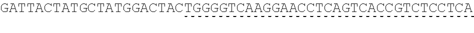

Heavy chain variable region: Amino acid sequence (140 AA) (SEQ ID NO: 68)
MERHWIFLFLLSVTAGVHSQVQLQQSAAELARPGASVKMSCKASGYTFSSYTMHWVKQRPG
QGLEWIGYINPSSGYTDYNQKFKDKTTLTADKSSSTAYMQLSSLTSEDSAVYYCARLYDNY

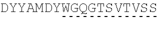

Light chain variable region: DNA sequence (396 bp) (SEQ ID NO: 69)
ATGGATTCACAGGCCCAGGTTCTTATATTGCTGCTGCTATGGGTATCTGGTACCTGTGGGG
ACATTGTGATGTCACAGTCTCCATCCTCCCTGGCTGTGTCAGCAGGAGAGAAGGTCACTAT
GAGCTGCAAATCCAGTCAGAGTCTGCTCAATAGTAGAATCCGAAAGAACTACTTGGCTTGG
TACCAGCAGAAACCAGGGCAGTCTCCTAAACTGCTGATCTACTGGGCATCCACTAGGGAAT
CTGGGGTCCCTGATCGCTTCACAGGCAGTGGATCTGGGACAGATTTCACTCTCACCATCAG
CAGTGTGCAGGCTGATGACCTGGCAGTTTATTACTGCAAGCAATCTTATAATCTGCTCACG

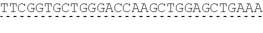

Light chain variable region: Amino acid sequence (132 AA) (SEQ ID NO: 70)
MDSQAQVLILLLLWVSGTCGDIVMSQSPSSLAVSAGEKVTMSCKSSQSLLNSRIRKNYLAW
YQQKPGQSPKLLIYWASTRESGVPDRFTGSGSGTDFTLTISSVQADDLAVYYCKQSYNLLT

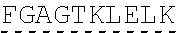

10E7E2 IgG1 GPV-P3 mAb sequencing results and analysis

Five single colonies with correct V_{H} and V_{L} insert sizes were sent for sequencing. The V_{H} and V_{L} genes of five different clones were found nearly identical. The consensus sequence, listed in Table 41, is the sequence of the antibody produced by the hybridoma 10E7E2.

TABLE 41
Heavy chain variable region: DNA sequence (405 bp) (SEQ ID NO: 71)
ATGGCTGTCTTGGGGCTGCTCTTCTGCCTGGTGACATTCCCAAGCTGTGTCCCATCCCAGG
TGCAGCTGAAGCAGTCAGGACCTGGCCTAGTGCAGCCCTCACAGAGCCTGTCCATCACCTG
CACAGTCTCTGGTTTCTCATTAACTAGCTATGGTGTACACTGGGTTCGCCAGTCTCCAGGA
AAGGGTCTGGAGTGGCTGGGAGTGATATGGAGTGGTGGAACTACAGACTATAATGCAGCTT
TCATATCCAGACTGAGCATCAGCAAGGACAATTCCAAGAGCCAAGTTTTCTTTAAAATGAA
CAGTCTGCAAGCTAATGACACAGCCATATATTACTGTGCCAGAGGGGGGCACCGAGGGTTT

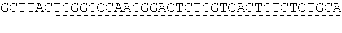

Heavy chain variable region: Amino acid sequence (135 AA) (SEQ ID NO: 72)
MAVLGLLFCLVTFPSCVPSQVQLKQSGPGLVQPSQSLSITCTVSGFSLTSYGVHWVRQSPG
KGLEWLGVIWSGGTTDYNAAFISRLSISKDNSKSQVFFKMNSLQANDTAIYYCARGGHRGF

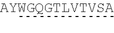

Light chain variable region: DNA sequence (393 bp) (SEQ ID NO: 73)
ATGGGCATCAAGATGGAGACACATTCTCAGGTCTTTGTATACATGTTGCTGTGGTTGTCTG
GTGTTGAAGGAGACATTGTGATGACCCAGTCTCACAAATTCATGTCCACATCAGTAGGAGA
CAGGGTCAGCATCACCTGCAAGGCCAGTCAAGATGTGGGTACTGCTGTAGCCTGGTATCAA
CAGAAACCAGGGCAATCTCCTAAACTACTGATTTACTGGGCATCCACCCGGCACACTGGAG
TCCCTGATCGCTTCACAGGCAGTGGATCTGGGACAGATTTCACTCTCACCATTAGCAATGT

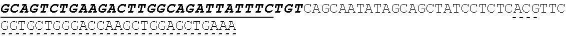

Light chain variable region: Amino acid sequence (131 AA) (SEQ ID NO: 74)
MGIKMETHSQVFVYMLLWLSGVEGDIVMTQSHKFMSTSVGDRVSITCKASQDVGTAVAWYQ

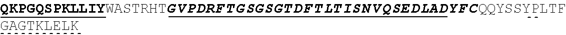

### Example 8

Signal peptide-FR1-CDR 1-FR2-CDR2-FR3-CDR3-FR4-CONSTANT REGION-STOP CODON

### Antibody Sequences of 7C6E8

TABLE 42
Heavy chain: DNA sequence (1407 bp) (SEQ ID NO: 238)
ATGGCTTGGGTGTGGACCTTGCTATTCCTGATGGCAGCTGCCCAAAGTATCCAAGCACAGA
TCCAGTTGGTGCAGTCTGGACCTGAGCTGAAGAAGCCTGGAGAGACAGTCAAGATCTCCTG
CAAGGCTTCTGGTTATACCTTCACAGACTATTCAATGCACTGGGTGAAGCAGACTCCAGGA
AAGGGTTTAAAGTGGATGGGCTGGATAAATACTGAGACTGGTGAGCCAACATATGCAGATG
ACTTCAAGGGACGGTTTGCCTTCTCTTTGGAAACCTCTGCCAGCACTGCCTATTTGCAGAT
CAACAACCTCAAAAATGAGGACACGGCTACATATTTCTGTGCTAGAAACTACTATGATACG

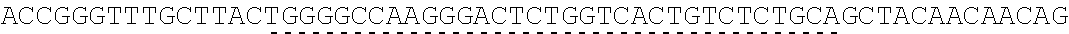

CCCCATCTGTCTATCCCTTGGTCCCTGGCTGCAGTGACACATCTGGATCCTCGGTGACACT
GGGATGCCTTGTCAAAGGCTACTTCCCTGAGCCGGTAACTGTAAAATGGAACTATGGAGCC
CTGTCCAGCGGTGTGCGCACAGTCTCATCTGTCCTGCAGTCTGGGTTCTATTCCCTCAGCA
GCTTGGTGACTGTACCCTCCAGCACCTGGCCCAGCCAGACTGTCATCTGCAACGTAGCCCA
CCCAGCCAGCAAGACTGAGTTGATCAAGAGAATCGAGCCTAGAATACCCAAGCCCAGTACC
CCCCCAGGTTCTTCATGCCCACCTGGTAACATCTTGGGTGGACCATCCGTCTTCATCTTCC
CCCCAAAGCCCAAGGATGCACTCATGATCTCCCTAACCCCCAAGGTTACGTGTGTGGTGGT
GGATGTGAGCGAGGATGACCCAGATGTCCATGTCAGCTGGTTTGTGGACAACAAAGAAGTA
CACACAGCCTGGACACAGCCCCGTGAAGCTCAGTACAACAGTACCTTCCGAGTGGTCAGTG
CCCTCCCCATCCAGCACCAGGACTGGATGAGGGGCAAGGAGTTCAAATGCAAGGTCAACAA
CAAAGCCCTCCCAGCCCCCATCGAGAGAACCATCTCAAAACCCAAAGGAAGAGCCCAGACA
CCTCAAGTATACACCATACCCCCACCTCGTGAACAAATGTCCAAGAAGAAGGTTAGTCTGA
CCTGCCTGGTCACCAACTTCTTCTCTGAAGCCATCAGTGTGGAGTGGGAAAGGAACGGAGA
ACTGGAGCAGGATTACAAGAACACTCCACCCATCCTGGACTCAGATGGGACCTACTTCCTC
TACAGCAAGCTCACTGTGGATACAGACAGTTGGTTGCAAGGAGAAATTTTTACCTGCTCCG
TGGTGCATGAGGCTCTCCATAACCACCACACACAGAAGAACCTGTCTCGCTCCCCTGGTAA
ATGA
Heavy chain: Amino acid sequence (468 AA) (SEQ ID NO: 239)
MAWVWTLLFLMAAAQSIQAQIQLVQSGPELKKPGETVKISCKASGYTFTDYSMHWVKQTPG
KGLKWMGWINTETGEPTYADDFKGRFAFSLETSASTAYLQINNLKNEDTATYFCARNYYDT

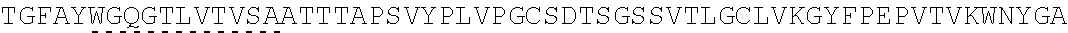

LSSGVRTVSSVLQSGFYSLSSLVTVPSSTWPSQTVICNVAHPASKTELIKRIEPRIPKPST
PPGSSCPPGNILGGPSVFIFPPKPKDALMISLTPKVTCVVVDVSEDDPDVHVSWFVDNKEV
HTAWTQPREAQYNSTFRVVSALPIQHQDWMRGKEFKCKVNNKALPAPIERTISKPKGRAQT
PQVYTIPPPREQMSKKKVSLTCLVTNFFSEAISVEWERNGELEQDYKNTPPILDSDGTYFL
YSKLTVDTDSWLQGEIFTCSVVHEALHNHHTQKNLSRSPGK-
Light chain 1: DNA sequence (717 bp) (SEQ ID NO: 240)
ATGAGTCCTGCCCAGTTCCTGTTTCTGTTAGTGCTCTGGATTCAGGAAACCAAAGGTGATG
TTGTGATGACCCAGACTCCACTCACTTTGTCGGTTACCGTTGGACAACCAGCCTCTATCTC
TTGCAAGTCAAGTCAGCGCCTCTTATATAGTTATGGAAAAACCTATTTGAATTGGTTATTA
CAGAGGCCAGGCCAGTCTCCAAAGCGCCTAATCTATCTGGTGTCTAAACTGGACTCTGGAG
TCCCTGACAGGTTCACTGGCAGTGGATCAGGAACAGATTTTACACTGAAAATCAGCAGAGT

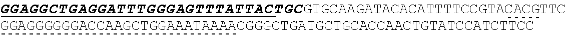

CACCATCCAGTGAGCAGTTAACATCTGGAGGTGCCTCAGTCGTGTGCTTCTTGAACAACTT
CTACCCCAAAGACATCAATGTCAAGTGGAAGATTGATGGCAGTGAACGACAAAATGGCGTC
CTGAACAGTTGGACTGATCAGGACAGCAAAGACAGCACCTACAGCATGAGCAGCACCCTCA
CGTTGACCAAGGACGAGTATGAACGACATAACAGCTATACCTGTGAGGCCACTCACAAGAC
ATCAACTTCACCCATTGTCAAGAGCTTCAACAGGAATGAGTGTTAG
Light chain 1: Amino acid sequence (238 AA) (SEQ ID NO: 241)
MSPAQFLFLLVLWIQETKGDVVMTQTPLTLSVTVGQPASISCKSSQRLLYSYGKTYLNWLL

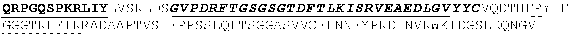

LNSWTDQDSKDSTYSMSSTLTLTKDEYERHNSYTCEATHKTSTSPIVKSFNRNEC-
Light chain 2: DNA sequence (708 bp) (SEQ ID NO: 242)
ATGGTATCCACACCTCAGTTCCTTGTATTTTTGCTTTTCTGGATTCCAGCCTCCAGAGGTG
ACATCTTGCTGACTCAGTCTCCAGCCATCCTGTCTGTGAGTCCAGGAGAAAGAGTCAGTTT
CTCCTGCAGGGCCAGTCAGAGCATTGGCACAAGCATACACTGGTATCAGCAAAGAACGAAT
GGTTCTCCAAGGCTTCTCATAAAGTTTGCTTCTGAGTCTATCTCTGGGATCCCTTCCAGGT
TTAGTGGCAGTGGATCAGGGACAGATTTTCCTCTTAGCATCAACAGTGTGGAGTCTGAAGA

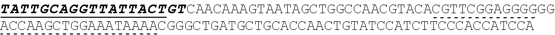

GTGAGCAGTTAACATCTGGAGGTGCCTCAGTCGTGTGCTTCTTGAACAACTTCTACCCCAA
AGACATCAATGTCAAGTGGAAGATTGATGGCAGTGAACGACAAAATGGCGTCCTGAACAGT
TGGACTGATCAGGACAGCAAAGACAGCACCTACAGCATGAGCAGCACCCTCACGTTGACCA
AGGACGAGTATGAACGACATAACAGCTATACCTGTGAGGCCACTCACAAGACATCAACTTC
ACCCATTGTCAAGAGCTTCAACAGGAATGAGTGTTAG
Light chain 2: Amino acid sequence (235 AA) (SEQ ID NO: 243)
MVSTPQFLVFLLFWIPASRGDILLTQSPAILSVSPGERVSFSCRASQSIGTSIHWYQQRTN

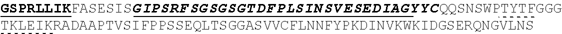

WTDQDSKDSTYSMSSTLTLTKDEYERHNSYTCEATHKTSTSPIVKSFNRNEC-

### Antibody Sequences of 9F2G5

TABLE 43
Heavy chain: DNA sequence (1389 bp) (SEQ ID NO: 244)
ATGGGATGGAGCTGGATCTTTCTCTTTCTCCTGTCAGGAACTGCAGGTGTCCTCTCTGAGG
TCCACCTGCAACAGTCTGGACCTGAGCTGGTGAAGCCTGGGGCTTCAGTGAAGATATCCTG
CAAGACTTCTGGATACATATTCACTGAATACACCATGCACTGGGTGAAGCAGAGCCATGGA
AAGAGCCTTGAGTGGATTGGAGGTATTAATCCTAACAATGGTGGTACTAGGTACAACCAGA
AGTTCAAGGGCAAGGCCACATTGACTGTAGACAAGTCCTCCAGCACAGCCTACATGGAGCT

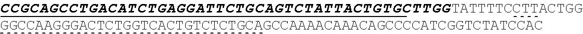

TGGCCCCTGTGTGTGGAGATACAACTGGCTCCTCGGTGACTCTAGGATGCCTGGTCAAGGG
TTATTTCCCTGAGCCAGTGACCTTGACCTGGAACTCTGGATCCCTGTCCAGTGGTGTGCAC
ACCTTCCCAGCTGTCCTGCAGTCTGACCTCTACACCCTCAGCAGCTCAGTGACTGTAACCT
CGAGCACCTGGCCCAGCCAGTCCATCACCTGCAATGTGGCCCACCCGGCAAGCAGCACCAA
GGTGGACAAGAAAATTGAGCCCAGAGGGCCCACAATCAAGCCCTGTCCTCCATGCAAATGC
CCAGCACCTAACCTCTTGGGTGGACCATCCGTCTTCATCTTCCCTCCAAAGATCAAGGATG
TACTCATGATCTCCCTGAGCCCCATAGTCACATGTGTGGTGGTGGATGTGAGCGAGGATGA
CCCAGATGTCCAGATCAGCTGGTTTGTGAACAACGTGGAAGTACACACAGCTCAGACACAA
ACCCATAGAGAGGATTACAACAGTACTCTCCGGGTGGTCAGTGCCCTCCCCATCCAGCACC
AGGACTGGATGAGTGGCAAGGAGTTCAAATGCAAGGTCAACAACAAAGACCTCCCAGCGCC
CATCGAGAGAACCATCTCAAAACCCAAAGGGTCAGTAAGAGCTCCACAGGTATATGTCTTG
CCTCCACCAGAAGAAGAGATGACTAAGAAACAGGTCACTCTGACCTGCATGGTCACAGACT
TCATGCCTGAAGACATTTACGTGGAGTGGACCAACAACGGGAAAACAGAGCTAAACTACAA
GAACACTGAACCAGTCCTGGACTCTGATGGTTCTTACTTCATGTACAGCAAGCTGAGAGTG
GAAAAGAAGAACTGGGTGGAAAGAAATAGCTACTCCTGTTCAGTGGTCCACGAGGGTCTGC
ACAATCACCACACGACTAAGAGCTTCTCCCGGACTCCGGGTAAATGA
Heavy chain: Amino acid sequence (462 AA) (SEQ ID NO: 245)
MGWSWIFLFLLSGTAGVLSEVHLQQSGPELVKPGASVKISCKTSGYIFTEYTMHWVKQSHG

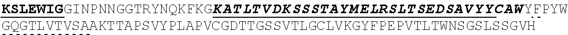

TFPAVLQSDLYTLSSSVTVTSSTWPSQSITCNVAHPASSTKVDKKIEPRGPTIKPCPPCKC
PAPNLLGGPSVFIFPPKIKDVLMISLSPIVTCVVVDVSEDDPDVQISWFVNNVEVHTAQTQ
THREDYNSTLRVVSALPIQHQDWMSGKEFKCKVNNKDLPAPIERTISKPKGSVRAPQVYVL
PPPEEEMTKKQVTLTCMVTDFMPEDIYVEWTNNGKTELNYKNTEPVLDSDGSYFMYSKLRV
EKKNWVERNSYSCSVVHEGLHNHHTTKSFSRTPGK-
Light chain: DNA sequence (717 bp) (SEQ ID NO: 246)
ATGAGTCCTGCCCAGTTCCTGTTTCTGTTAGTGCTCTGGATTCGGGAAACCAACGGTGATG
TTGTGATGACCCAGACTCCACTCACTTTGTCGGTTACCCTTGGACAACCAGCCTCCATCTC
TTGCAGGTCAAGTCAGAGCCTCTTAGATAGTGATGGAAAGACATATTTGAATTGGTTGTTA
CAGAGGCCAGGCCAGTCTCCAAAGCGCCTAATCTATCTGGTGTCTAAACTGGACTCTGGAG
TCCCTGACAGGTTCACTGGCAGTGGATCAGGGACAGATTTCACACTGAAAATCAGCAGAGT

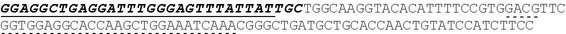

CACCATCCAGTGAGCAGTTAACATCTGGAGGTGCCTCAGTCGTGTGCTTCTTGAACAACTT
CTACCCCAAAGACATCAATGTCAAGTGGAAGATTGATGGCAGTGAACGACAAAATGGCGTC
CTGAACAGTTGGACTGATCAGGACAGCAAAGACAGCACCTACAGCATGAGCAGCACCCTCA
CGTTGACCAAGGACGAGTATGAACGACATAACAGCTATACCTGTGAGGCCACTCACAAGAC
ATCAACTTCACCCATTGTCAAGAGCTTCAACAGGAATGAGTGTTAG
Light chain: Amino acid sequence (238 AA) (SEQ ID NO: 247)
MSPAQFLFLLVLWIRETNGDVVMTQTPLTLSVTLGQPASISCRSSQSLLDSDGKTYLNWLL

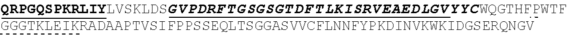

LNSWTDQDSKDSTYSMSSTLTLTKDEYERHNSYTCEATHKTSTSPIVKSFNRNEC-

### Antibody Sequences of 6H1G8

TABLE 44
Heavy chain: DNA sequence (1431 bp) (SEQ ID NO: 248)
ATGGGATGGAGCTGGATCTTTCTCTTTCTCCTGTCAGGAACTGCAGGTGTCCTCTCTGAGG
TCCAGCTGCAACAGTCTGGACCTGAGCTGGTGAAGCCTGGGGCCTCAGTGAAGATATCCTG
CAAGGCTTCTGGTTACTCAATCACTGGCTACTACATGCACTGGGTGAAGCAAAGCCATGTA
AAGAGCCTTGAGTGGATTGGACGTATTAATCCTTACACTGGTGCTACTAGCTACACCCAGA
ATTTCAAGGACAAGGCCAGCTTGACTGTAGATAAGTCCTCCAGCACAGCCTACATGGAGCT
CCTCAGCCTGAAATCTGAAGACTCTGCAGTCTATTACTGTGCGAAGGGGGGCGGTAGGAGC

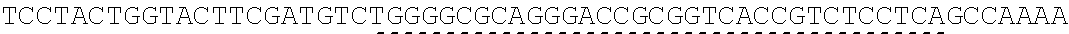

CAACACCCCCATCAGTCTATCCACTGGCCCCTGGGTGTGGAGATACAACTGGTTCCTCCGT
GACTCTGGGATGCCTGGTCAAGGGCTACTTCCCTGAGTCAGTGACTGTGACTTGGAACTCT
GGATCCCTGTCCAGCAGTGTGCACACCTTCCCAGCTCTCCTGCAGTCTGGACTCTACACTA
TGAGCAGCTCAGTGACTGTCCCCTCCAGCACCTGGCCAAGTCAGACCGTCACCTGCAGCGT
TGCTCACCCAGCCAGCAGCACCACGGTGGACAAAAAACTTGAGCCCAGCGGGCCCATTTCA
ACAATCAACCCCTGTCCTCCATGCAAGGAGTGTCACAAATGCCCAGCTCCTAACCTCGAGG
GTGGACCATCCGTCTTCATCTTCCCTCCAAATATCAAGGATGTACTCATGATCTCCCTGAC
ACCCAAGGTCACGTGTGTGGTGGTGGATGTGAGCGAGGATGACCCAGACGTCCAGATCAGC
TGGTTTGTGAACAACGTGGAAGTACACACAGCTCAGACACAAACCCATAGAGAGGATTACA
ACAGTACTATCCGGGTGGTCAGCACCCTCCCCATCCAGCACCAGGACTGGATGAGTGGCAA
GGAGTTCAAATGCAAGGTCAACAACAAAGACCTCCCATCACCCATCGAGAGAACCATCTCA
AAAATTAAAGGGCTAGTCAGAGCTCCACAAGTATACATCTTGCCGCCACCAGCAGAGCAGT
TGTCCAGGAAAGATGTCAGTCTCACTTGCCTGGTCGTGGGCTTCAACCCTGGAGACATCAG
TGTGGAGTGGACCAGCAATGGGCATACAGAGGAGAACTACAAGGACACCGCACCAGTCCTG
GACTCTGACGGTTCTTACTTCATATATAGCAAGCTCAATATGAAAACAAGCAAGTGGGAGA
AAACAGATTCCTTCTCATGCAACGTGAGACACGAGGGTCTGAAAAATTACTACCTGAAGAA
GACCATCTCCCGGTCTCCGGGTAAATGA
Heavy chain: Amino acid sequence (476 AA) (SEQ ID NO: 249)
MGWSWIFLFLLSGTAGVLSEVQLQQSGPELVKPGASVKISCKASGYSITGYYMHWVKQSHV
KSLEWIGRINPYTGATSYTQNFKDKASLTVDKSSSTAYMELLSLKSEDSAVYYCAKGGGRS

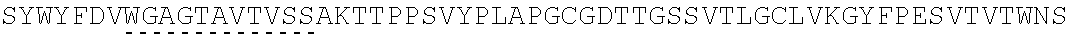

GSLSSSVHTFPALLQSGLYTMSSSVTVPSSTWPSQTVTCSVAHPASSTTVDKKLEPSGPIS
TINPCPPCKECHKCPAPNLEGGPSVFIFPPNIKDVLMISLTPKVTCVVVDVSEDDPDVQIS
WFVNNVEVHTAQTQTHREDYNSTIRVVSTLPIQHQDWMSGKEFKCKVNNKDLPSPIERTIS
KIKGLVRAPQVYILPPPAEQLSRKDVSLTCLVVGFNPGDISVEWTSNGHTEENYKDTAPVL
DSDGSYFIYSKLNMKTSKWEKTDSFSCNVRHEGLKNYYLKKTISRSPGK-
Light chain: DNA sequence (702 bp) (SEQ ID NO: 250)
ATGTCCTCTGCTCAGTTCCTTGGTCTCCTATTGCTCTGTTTTCAAGGTACCAGATGTGATA
TCCAGATGACACAGACTACATCCTCCCTGTCTGCCTCTCTGGGAGACAGAGTCACCATCAG
TTGCAGTGCAAGTCAGGCCATTAGCAATTATTTAAACTGGTATCAGCAGAAACCAGATGGA
ACTATTAAACTCCTGATCTATTACACATCAAGTTTACACTCAGGAGTCCCATCAAGGTTCA
GTGGCAGTGGGTCTGGGACAGATTATTCTCTCACCATCAGCAACCTGGAACCTGAAGATAT

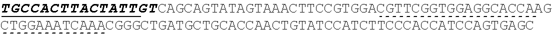

AGTTAACATCTGGAGGTGCCTCAGTCGTGTGCTTCTTGAACAACTTCTACCCCAAAGACAT
CAATGTCAAGTGGAAGATTGATGGCAGTGAACGACAAAATGGCGTCCTGAACAGTTGGACT
GATCAGGACAGCAAAGACAGCACCTACAGCATGAGCAGCACCCTCACGTTGACCAAGGACG
AGTATGAACGACATAACAGCTATACCTGTGAGGCCACTCACAAGACATCAACTTCACCCAT
TGTCAAGAGCTTCAACAGGAATGAGTGTTAG
Light chain: Amino acid sequence (233 AA) (SEQ ID NO: 251)
MSSAQFLGLLLLCFQGTRCDIQMTQTTSSLSASLGDRVTISCSASQAISNYLNWYQQKPDG

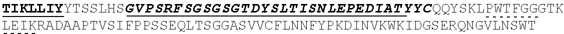

DQDSKDSTYSMSSTLTLTKDEYERHNSYTCEATHKTSTSPIVKSFNRNEC-

### Antibody Sequences of 7H6A2

TABLE 45
Heavy chain: DNA sequence (1419 bp) (SEQ ID NO: 252)
ATGGCTTGGGTGTGGACCTTGCTATTCCTGATGGCAGCTGCCCAAAGTATCCAAGCACAGA
TCCAGTTGGTGCAGTCTGGACCTGAGCTGAAGAAGCCTGGAGAGACAGTCAAGATCTCCTG
CAAGGCTTCTGGTTATACCTTCACAGACTTTTCAATGCACTGGGTGAAGCAGGCTCCAGGA
AAGGGTTTAAAGTGGATGGGCTGGATAAACACTGAGACTGTTGAGCCAACATATGCAGATG
ACTTCAAGGGACGGTTTGCCTTCTCTTTGGAAACCTCTGCCAGCACTGCCTATTTGCAGAT
CAACAACCTCAAAAATGAGGACACGGCTACATATTTCTGTTCTAGAGTCTCCTATGATGGT

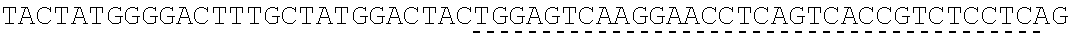

CCAAAACAACAGCCCCATCGGTCTATCCACTGGCCCCTGTGTGTGGAGATACAACTGGCTC
CTCGGTGACTCTAGGATGCCTGGTCAAGGGTTATTTCCCTGAGCCAGTGACCTTGACCTGG
AACTCTGGATCCCTGTCCAGTGGTGTGCACACCTTCCCAGCTGTCCTGCAGTCTGACCTCT
ACACCCTCAGCAGCTCAGTGACTGTAACCTCGAGCACCTGGCCCAGCCAGTCCATCACCTG
CAATGTGGCCCACCCGGCAAGCAGCACCAAGGTGGACAAGAAAATTGAGCCCAGAGGGCCC
ACAATCAAGCCCTGTCCTCCATGCAAATGCCCAGCACCTAACCTCTTGGGTGGACCATCCG
TCTTCATCTTCCCTCCAAAGATCAAGGATGTACTCATGATCTCCCTGAGCCCCATAGTCAC
ATGTGTGGTGGTGGATGTGAGCGAGGATGACCCAGATGTCCAGATCAGCTGGTTTGTGAAC
AACGTGGAAGTACACACAGCTCAGACACAAACCCATAGAGAGGATTACAACAGTACTCTCC
GGGTGGTCAGTGCCCTCCCCATCCAGCACCAGGACTGGATGAGTGGCAAGGAGTTCAAATG
CAAGGTCAACAACAAAGACCTCCCAGCGCCCATCGAGAGAACCATCTCAAAACCCAAAGGG
TCAGTAAGAGCTCCACAGGTATATGTCTTGCCTCCACCAGAAGAAGAGATGACTAAGAAAC
AGGTCACTCTGACCTGCATGGTCACAGACTTCATGCCTGAAGACATTTACGTGGAGTGGAC
CAACAACGGGAAAACAGAGCTAAACTACAAGAACACTGAACCAGTCCTGGACTCTGATGGT
TCTTACTTCATGTACAGCAAGCTGAGAGTGGAAAAGAAGAACTGGGTGGAAAGAAATAGCT
ACTCCTGTTCAGTGGTCCACGAGGGTCTGCACAATCACCACACGACTAAGAGCTTCTCCCG
GACTCCGGGTAAATGA
Heavy chain: Amino acid sequence (472 AA) (SEQ ID NO: 253)
MAWVWTLLFLMAAAQSIQAQIQLVQSGPELKKPGETVKISCKASGYTFTDFSMHWVKQAPG
KGLKWMGWINTETVEPTYADDFKGRFAFSLETSASTAYLQINNLKNEDTATYFCSRVSYDG

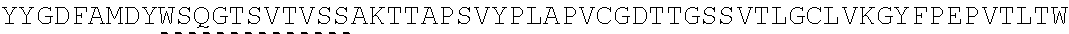

NSGSLSSGVHTFPAVLQSDLYTLSSSVTVTSSTWPSQSITCNVAHPASSTKVDKKIEPRGP
TIKPCPPCKCPAPNLLGGPSVFIFPPKIKDVLMISLSPIVTCVVVDVSEDDPDVQISWFVN
NVEVHTAQTQTHREDYNSTLRVVSALPIQHQDWMSGKEFKCKVNNKDLPAPIERTISKPKG
SVRAPQVYVLPPPEEEMTKKQVTLTCMVTDFMPEDIYVEWTNNGKTELNYKNTEPVLDSDG
SYFMYSKLRVEKKNWVERNSYSCSVVHEGLHNHHTTKSFSRTPGK-
Light chain: DNA sequence (705 bp) (SEQ ID NO: 254)
ATGATGTCCTCTGCTCAGTTCCTTGGTCTCCTGTTGCTCTGTTTTCAAGGTACCAGATGTG
ATATCCAGATGACACAGACTACATCCTCCCTGTCTGCCTCTCTGGGAGACAGAGTCACCAT
CAGTTGCAGGGCAAGTCAGGACATTAGCAATTATTTAAACTGGTATCAGCAGAAACCAGAT
GGAACTGTTAAACTCCTGATCTACTACACATCAAGATTACACTCAGGAGTCCCATCAAGGT
TCAGTGGCAGTGGGTCTGGAACAGATTATTCTCTCACCATTAGCAACCTGGAGCAAGAAGA

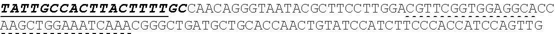

AGCAGTTAACATCTGGAGGTGCCTCAGTCGTGTGCTTCTTGAACAACTTCTACCCCAAAGA
CATCAATGTCAAGTGGAAGATTGATGGCAGTGAACGACAAAATGGCGTCCTGAACAGTTGG
ACTGATCAGGACAGCAAAGACAGCACCTACAGCATGAGCAGCACCCTCACGTTGACCAAGG
ACGAGTATGAACGACATAACAGCTATACCTGTGAGGCCACTCACAAGACATCAACTTCACC
CATTGTCAAGAGCTTCAACAGGAATGAGTGTTAG
Light chain: Amino acid sequence (234 AA) (SEQ ID NO: 255)
MMSSAQFLGLLLLCFQGTRCDIQMTQTTSSLSASLGDRVTISCRASQDISNYLNWYQQKPD

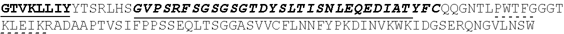

TDQDSKDSTYSMSSTLTLTKDEYERHNSYTCEATHKTSTSPIVKSFNRNEC-

### Example 9

### MAbs Provided Enhanced Resistance to Disseminated Candidiasis in Neutropenic Mice.

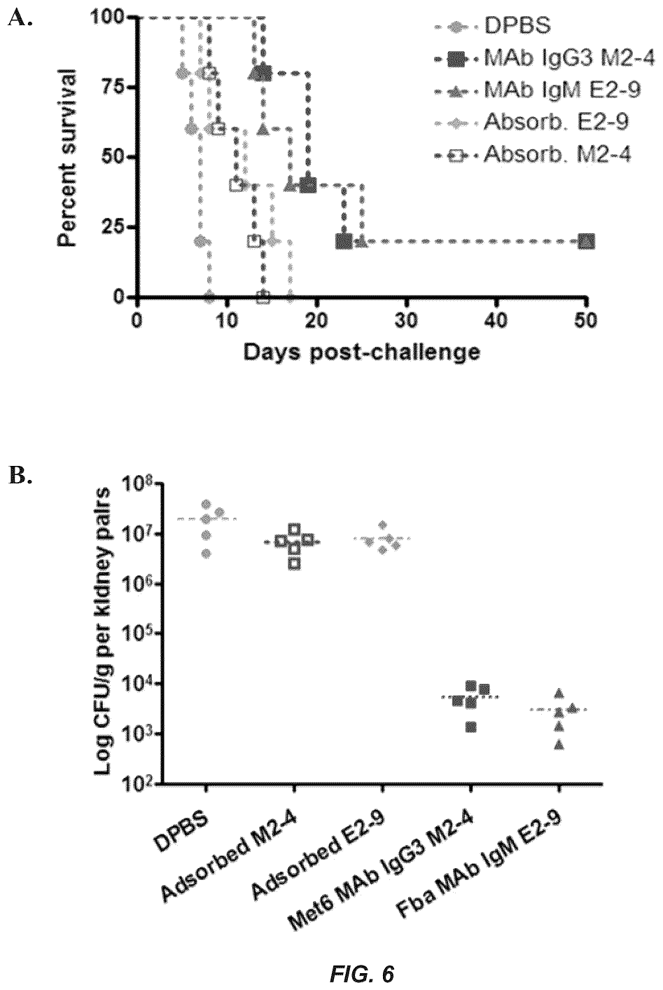

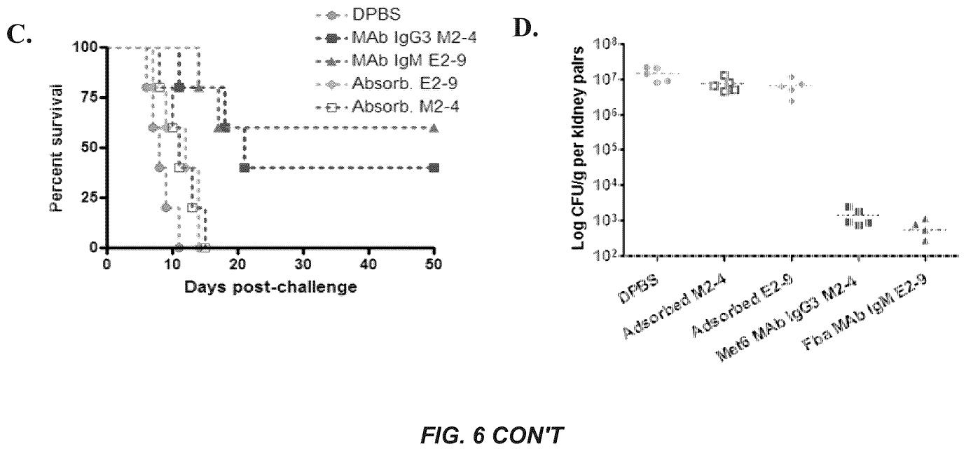

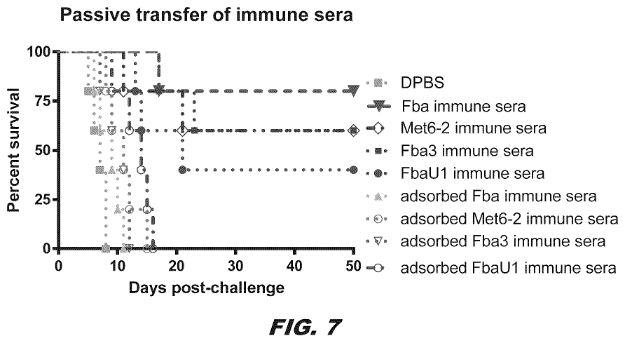

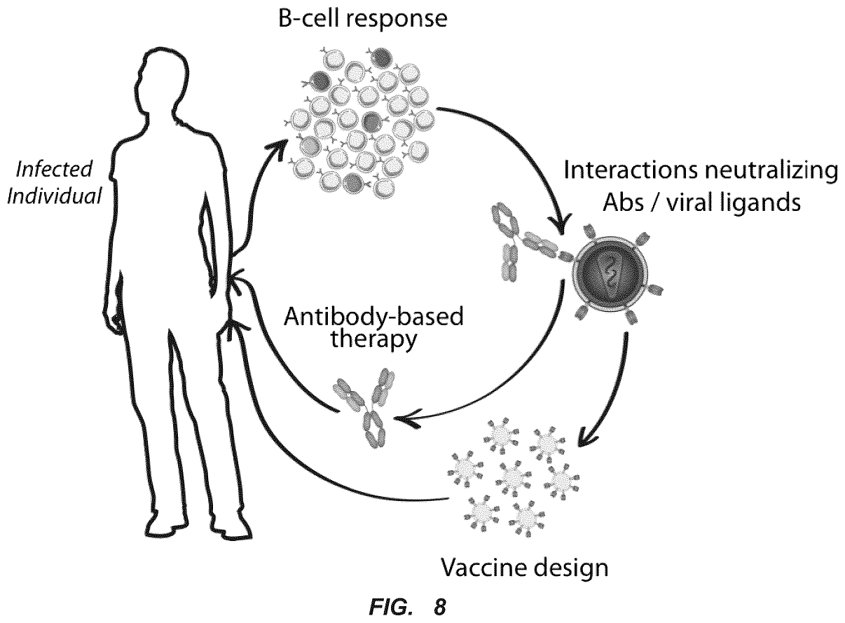

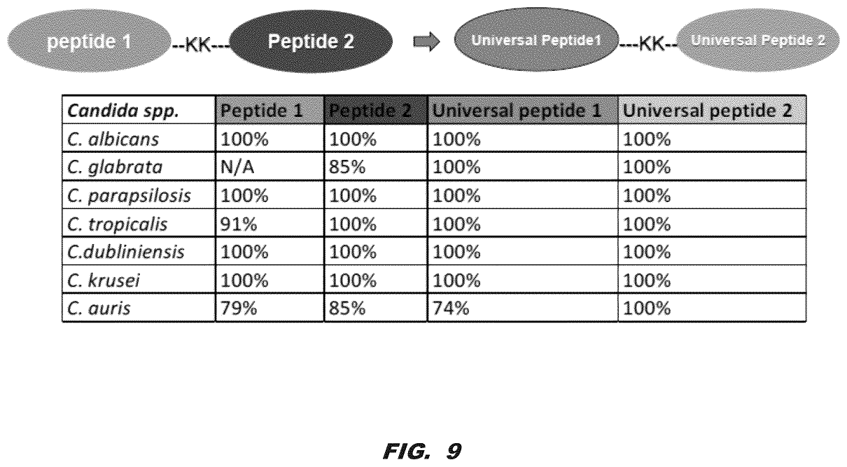

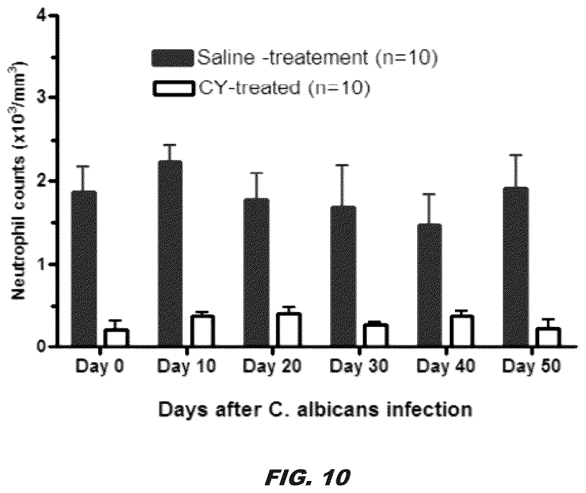

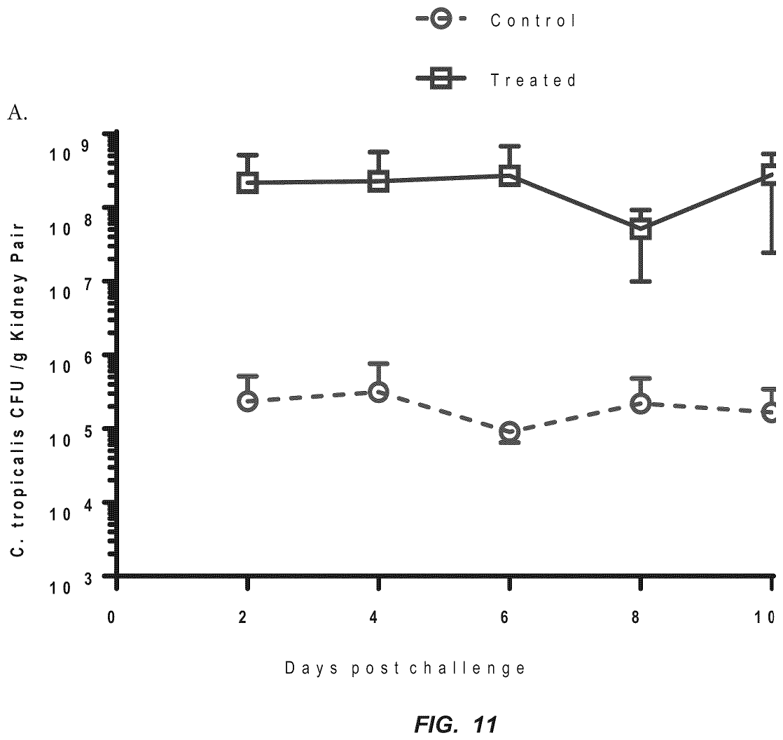

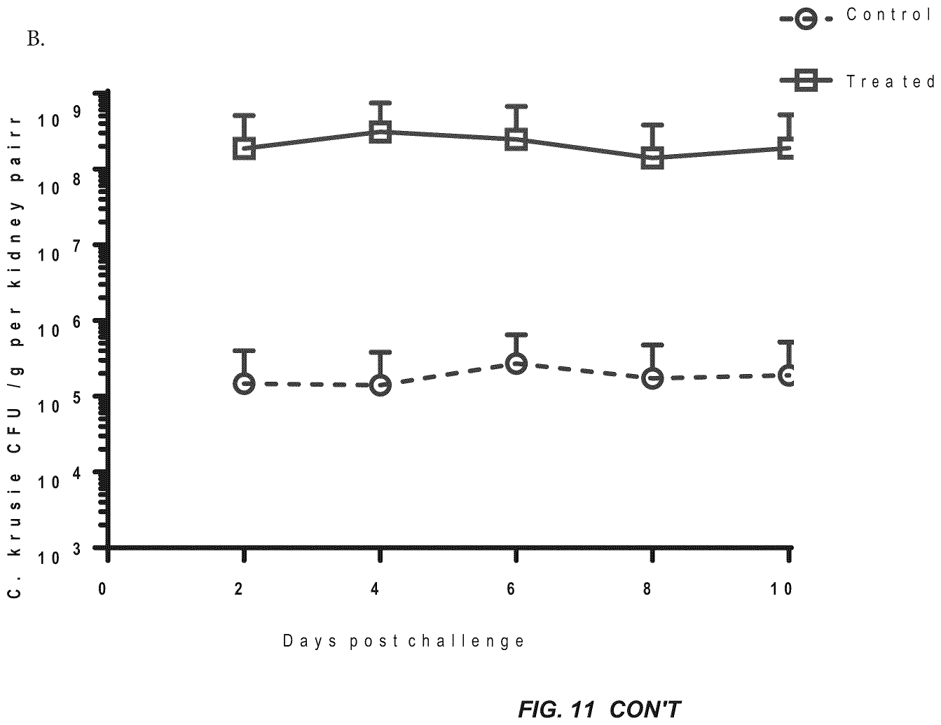

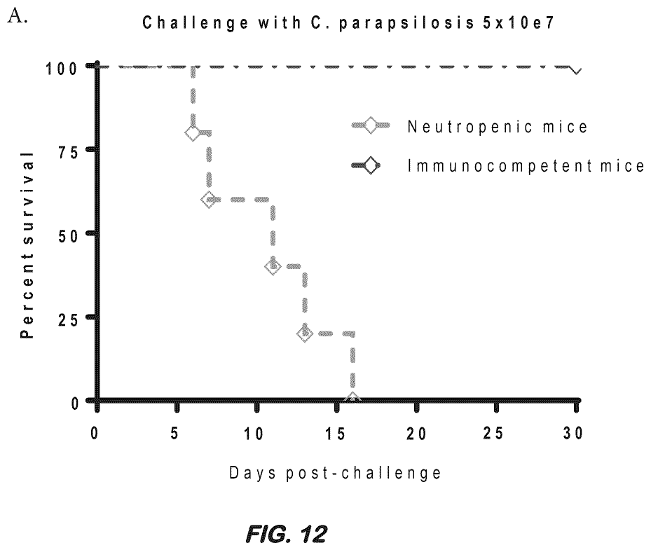

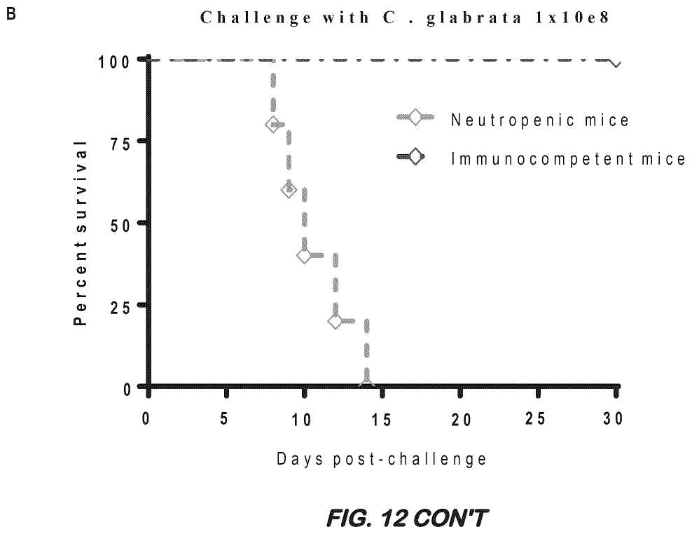

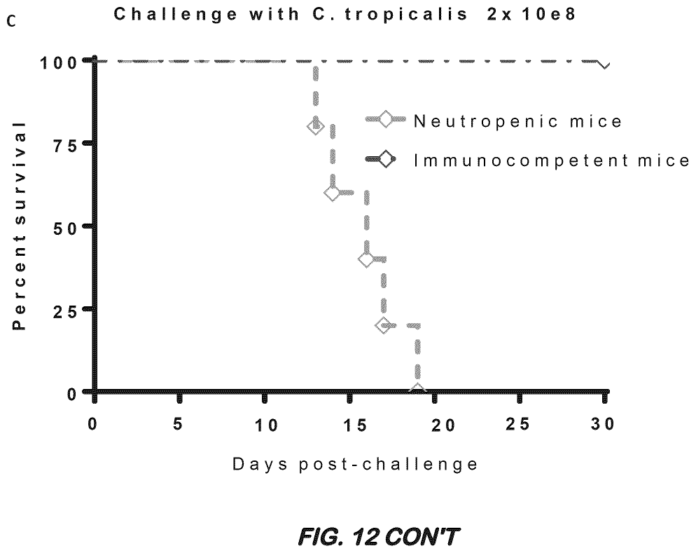

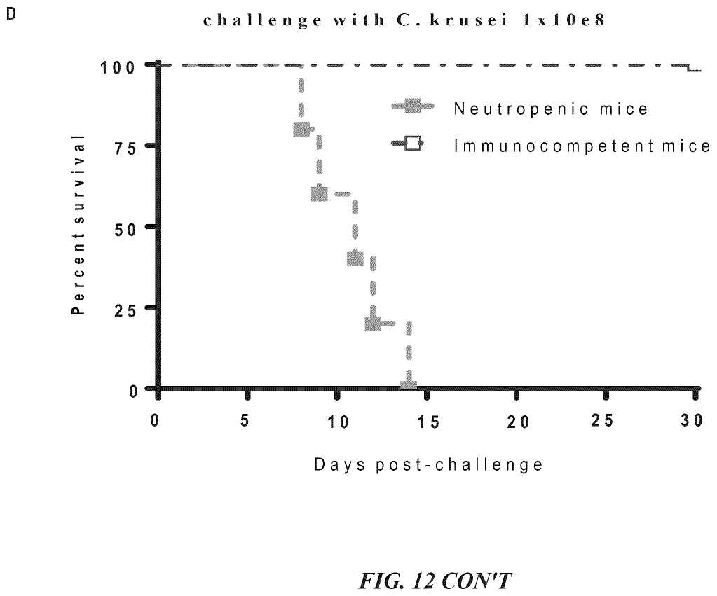

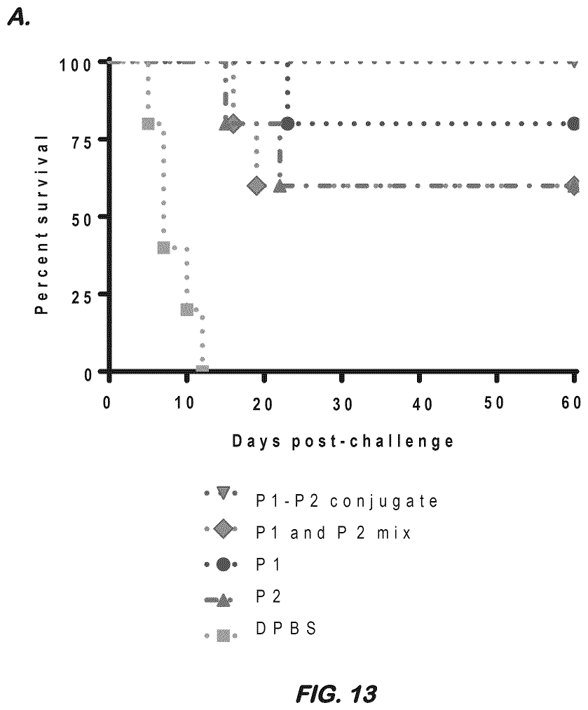

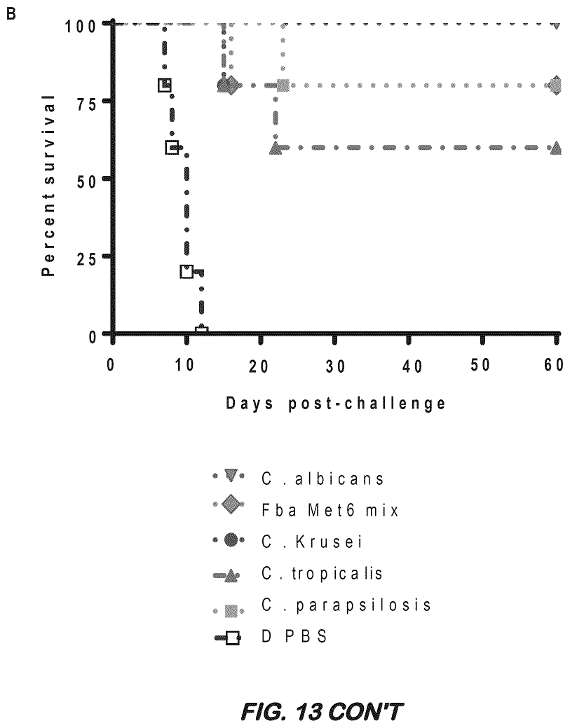

The possibility of using passive mAb immunotherapy in neutropenic mice can be evaluated. As an example, the protective effect of mAb specific for antigenic universal peptides against disseminated candidiasis in neutropenic mice can be examined by passive transfer experiments. To verify that the protection elicited by antibodies is indeed due to mAbs, control groups can receive mAbs absorbed with C. albicans yeast cells. For example, mice can receive either the mAb itself, in one dose or multiple doses during 14 day period, or absorbed mAbs. Without wishing to be bound by theory, it is expected that the group receiving treatment of protective mAb will have significantly reduced CFUs in kidneys compared with control mice. When each mAb is given every other day for two weeks, mAb is expected to be able to increase protection, such as from 20%0 to 4000, or from 2000 to 6000, with similar improved efficacy as we observed in immunocompetent mice. As an example, see FIG. 6.

### Example 10

MAbs have Therapeutic Activity for Disseminated Candidiasis.

The therapeutic efficacy of mAb, such as those specific for antigenic universal peptides, can be examined in the murine model of disseminated candidiasis. Without wishing to be bound by theory, it is expected that data resulting from the kidney-CFU measurement will show that the administration of such mAbs to BLAB/c mice at 1 h, 2h, 4h post-infection will exert a therapeutic potential to the disseminated disease, whereas administrations of mAbs at 8h, 12h or 24h post-infection will not be effective.

### Example 11

Combined two universal-peptide-specific mAbs (mAb cocktails) conferred enhanced protection against systemic candidiasis in passive transfer as compared to single mAb treatment.

TABLE 13

| | | | | | Protection |
|---|---|---|---|---|---|
| protective | | | | | of adsorbed |
| mAb | Control | control | Protection of | Synergy of | mAb cocktails |
| cocktails | #1 | #2 | mAb cocktails | combinations | or mAb |
| 2C9 + 1A11 | 2C9 | 1A11 | High (90-100%) | Yes++ | None |
| 2C9 + 10B6 | 2C9 | 10B6 | High (90-100%) | Yes++ | None |
| 2C9 + 5G5 | 2C9 | 5G5 | N/A | N/A | None |
| 2C9 + 10E7 | 2C9 | 10E7 | High (70-80%) | No | None |
| 5A9 + 10E7 | 5A9 | 10E7 | High (100%) | Yes++ | None |
| 5A9 + 1A11 | 5A9 | 1A11 | High (100%) | Yes++ | None |
| 5A9 + 10B6 | 5A9 | 10B6 | High (70-80%) | No | None |
| 5A9 + 5G5 | 5A9 | 5G5 | N/A | N/A | None |
| 6E3 + 10E7 | 6E3 | 10E7 | High (80-90%) | Yes | None |
| 6E3 + 10B6 | 6E3 | 10B6 | High (70-80%) | No | None |
| 6E3 + 1A11 | 6E3 | 1A11 | High (100%) | Yes+ + | None |
| 6E3 + 5G5 | 6E3 | 5G5 | N/A | N/A | None |
| 7A3 + 10E7 | 7A3 | 10E7 | High (70-80%) | No | None |
| 7A3 + 10B6 | 7A3 | 10B6 | High (90-100%) | Yes++ | None |
| 7A3 + 1A11 | 7A3 | 1A11 | High (90-100%) | Yes++ | None |
| 7A3 + 5G5 | 7A3 | 5G5 | High (90-100%) | Yes | None |

Passive protection against disseminated candidiasis caused by C. albicans was prevented by removal of the mAbs by absorption with Candida cells before transfer.

TABLE 14
Hybridoma clone, peptide vaccines, and related protective peptide-specific mAbs

| | | mAb | Peptide | | Protection of |
|---|---|---|---|---|---|
| Hybridomas | mAbs | isotype | vaccine | Protection | adsorbed mAb |
| 3H7E3 | 3H7 | IgG1 | Met6 peptide | High (70-90%) | None |
| | | | SEQ ID NO: 30 | | |
| 5E12D10 | 5E12 | IgG1 | Met6 peptide | High (90-100%) | None |
| | | | SEQ ID NO: 30 | | |
| 2B10C1 | 2B10 | IgG1 | Met6 peptide | High (80-100%) | None |
| | | | SEQ ID NO: 30 | | |
| 7H6A2 | 7H6 | IgG2b | Met6 peptide | High (80-90%) | None |
| | | | SEQ ID NO: 30 | | |
| 6H1G8 | 6H1 | IgG2b | Hwp1 peptide | High (70-80%) | None |
| | | | SEQ ID NO: 31 | | |
| 3B5H7 | 3B5 | IgG2b | Hwp1 peptide | High (70-80%) | None |
| | | | SEQ ID NO: 31 | | |
| 5B11H1 | 5B11 | IgG1 | Hwp1 peptide | High (90-100%) | None |
| | | | SEQ ID NO: 31 | | |
| 2D5F7 | 2D5 | IgG2b | Fba peptide | High (90-100%) | None |
| | | | SEQ ID NO: 29 | | |
| 3E2G2 | 3E2 | IgG2b | Fba peptide | High (70-90%) | None |
| | | | SEQ ID NO: 29 | | |
| 6G9E11 | 6G9 | IgG2b | Fba peptide | High (80-100%) | None |
| | | | SEQ ID NO: 29 | | |
| 9C11F6 | 9C11 | IgG2b | Fba peptide | High (90-100%) | None |
| | | | SEQ ID NO: 29 | | |
| 7H5H6 | 7H5 | IgG2b | Fba peptide | High (80-100%) | None |
| | | | SEQ ID NO: 29 | | |

Additional data for universal peptide-specific mAbs. Passive protection against disseminated candidiasis caused by C. albicans was prevented by removal of the mAbs by absorption with Candida cells before transfer.

TABLE 15
Hybridoma clone and related protective peptide-specific mAbs

| | | | | | Protection |
|---|---|---|---|---|---|
| | | mAb | peptide | | of adsorbed |
| Hybridomas | mAb | isotype | vaccine | Protection | mAb |
| 2C9G3 | 2C9 | IgG2a | GPV-M2 universal | High (80-100%) | None |
| | | | SEQ ID NO: 2 | | |
| 5A9D1 | 5A9 | IgG3 | GPV-M2 universal | High (80-90%) | None |
| | | | SEQ ID NO: 2 | | |
| 5A11B1 | 5A1 | IgG3 | GPV-M2 universal | High (60-90%) | None |
| | | | SEQ ID NO: 2 | | |
| 6E3D9 | 6E3 | IgG1 | GPV-M1 universal | High (80-100%) | None |
| | | | SEQ ID NO: 1 | | |
| 1D4H5 | 1D5 | IgG1 | GPV-M1 universal | High (70-80%) | None |
| | | | SEQ ID NO: 1 | | |
| 7A3C4 | 7A3 | IgG1 | GPV-M1 universal | High (70-90%) | None |
| | | | SEQ ID NO: 1 | | |
| 10E7E2 | 10E7 | IgG1 | GPV-P3 universal | High (80-100%) | None |
| | | | SEQ ID NO: 16 | | |
| 1A11H8 | 1A11 | IgG3 | GPV-P1 universal | High (60-90%) | None |
| | | | SEQ ID NO: 14 | | |
| 10B6H8 | 10B6 | IgG1 | GPV-P1 universal | High (80-100%) | None |
| | | | SEQ ID NO: 14 | | |
| 5G5G3 | 5G5 | IgG2a | GPV-P1 universal | High (80-100%) | None |
| | | | SEQ ID NO: 14 | | |

### Example 12

### Abstract

Candida species are a major cause of fungal infections, yet to date there are no vaccines against Candida or indeed any other fungal pathogen. Our knowledge of immunity to Candida has almost exclusively been obtained from studies on Candida albicans, the most common disease-causing species. However, non-albicans Candida (NAC) species also cause disease and their prevalence is increasing. Worryingly, antifungal drug resistance has been detected for all clinically relevant Candida species to some degree. Research into immunity to NAC species is still at an early stage due to the lack of tractable animal models with which to study these important pathogens. This is partly because many NAC species are not usually pathogenic in mouse models of candidiasis. Therefore, developing a faithful murine model to understand immunity to NAC species is key for future studies in this area.

The purpose of this study was to 1, perform a simultaneous comparison of the relative pathogenicity of the five clinically relevant Candida species in both immunocompetent and immunocompromised mouse models in order to present an overall picture of species-related variations in virulence. 2, to establish neutropenic mouse models of disseminated candidiasis by several medically important NAC species. 3, to address the fundamental question as to whether the peptide vaccine(s) will protect neutropenic mice from disseminated candidiasis caused by medically important NAC species. As results, we have successfully established a NAC invasive infection model in immunosuppressed BALB/c mice induced by cyclophosphamide, including C. tropicalis, C. glabrata, C. parapsilosis and C. krusei. Together with C. albicans, these four NAC species cause more than 90% of invasive infections. The intravenous (IV) challenge mouse model has been used to compare the virulence of different Candida species. The survival study was performed by comparing mortality and degree of kidney infection. Importantly, we demonstrated the protective efficacy of two peptide vaccines, which protect mice against disseminated candidiasis caused by C. albicans, also protected neutropenic mice against other medical significant NAC species.

Vaccinated BALB c Mice were Rendered Severely Neutropenic (Polymorphonuclear Leukocyte Count, <500 mm3) 3 Days Prior to the Beginning of the Challenge Study (Day 0) with 200 mg/kg of Body Weight Intraperitoneal (i.p.) Cyclophosphamide (CY, Sigma).

Significant decreases in the numbers of total leukocytes and neutrophils were observed in the CY-treated mice compared with those in the control mice. The effects of CY treatment were monitored every 2-3 days during the entire experimental procedure. The total neutrophil counts were reduced (<500 cells/mm3) within 3 days of the first CY injection, and severe neutropenia was maintained until the termination of the experiments at day 50.

Effect of immunosuppression on kidney fungal burdens of immunosuppressed mice infected with C. tropicalis and C. krusei. Mice were inoculated with 2×10⁸ CFU of C. tropicalis per mouse. Twenty-four hours before fungal inoculation, one group of mice was immunosuppressed with an intraperitoneal injection of 200 mg/kg of cyclosphamide (CY).

Statistically significant differences were seen between control (DPBS i.p.) and immunosuppressed animals (p<0.01). B. Mice were inoculated with 1×10⁸ CFU of C. Krusei per animal. Twenty-four hours before fungal inoculation, one group of mice was immuno-suppressed with an intraperitoneal injection of 200 mg/kg of CY. Statistically significant differences were seen between normal control and immuno-suppressed animals (p<0.01). We have seen a similar effect of immunosuppression on kidney CFU with other NAC species.

### Establish Neutropenic Mouse Models of NAC

Neutropenic murine models of disseminated infection by C. tropicalis, C. glabrata, C. parapsilosis and C. Krusei have been successfully established. To induce neutropenia, naïve mice received a 200 mg/kg dose of cyclophosphamide (CY) by i.p. on day −3. Prolonged neutropenia was maintained for 30-50 days by giving each animal a 150 mg/kg dose of CY by i.p. every 10 days after infection. On day 0, mice of different experimental groups (five mice per group) were intravenously infected with 5×107 viable C. parapsilosis ATCC MYA-4646 (A), 1×108 C. glabrata ATCC 2001 cells (B) and 2×108 C. tropicalis ATCC 28775 cells (C) or 1×108 C. Krusei ATCC 32196 in 0.1 ml DPBS (D). As controls, immunocompetent mice were challenged with the same dose of each Candida strain tested.

### Double Peptide Vaccines Induced Solid Protection Against Disseminated Candidiasis in Mice.

A. P1 peptide was conjugated to the N terminus of P2 peptide through double lysine linker to form P1-P2 conjugate. Mice immunized with P1, P2 or P1 & P2 mixture were used as positive controls to compare the efficacy between single peptide vaccine and double peptide conjugate. After animals were challenged, P1-P2 conjugate induced 100% complete protection up to 60 days when the experiment was terminated. Our data indicate P1-P2 double peptide vaccine worked superior as compared to other individual peptides or peptide mixture.

B. By the same approach, double universal peptide 1 & 2 (UP1-UP2) conjugate was used to vaccinate mice and evaluated for its ability to induce protective immunity against disseminated candidiasis caused by medically important non-albicans species. Immunized mice challenged with C. albicans were used as positive controls, and mice that received DPBS and challenged with C. albicans were used as negative controls. Survival data show that universal double peptide conjugate vaccine induced protective immunity against all the medically important non-albicans Candida species, in addition to C. albicans.

### Conclusions

Neutropenic murine models of disseminated infection by C. tropicalis, C. glabrata, C. Krusei and C. parapsilosis have been successfully established.

A double peptide conjugate vaccine, targeting two peptides expressed on the cell surface of all the medically important Candida species, can induce a high degree of protection against disseminated candidiasis in a mouse model of human disseminated candidiasis by Candida species.

### Example 13

mAbs Protect Against Candida Auris

Induction of immunosuppression in mice:

An immunocompromised murine model of disseminated infection by C. auris was developed. To induce immunosuppression, Jackson B6.129X1Elane mice received a 200 mg/kg dose of cyclophosphamide (CY) by i.p. on day −3. Prolonged immunosuppression was maintained for 30 days by giving each animal a 200 mg/kg dose of CY by i.p. every 10 days after infection. On day 0, mice of different experimental groups were intravenously infected with 2×108 viable C. auris yeast cells.

### Passive Transfer of MAbs by Intraperitoneal (i.p.) Route:

The preventive effect of peptide-specific mAbs, for example 7H6A2 and 1D4H5, and combinations thereof, for example 7H6A2+1D4H5, was examined by passive transfer experiments. Each mAb was appropriately diluted in DPBS to give a 40,000-100,000 ELISA titer against each corresponding specific peptide coated on the plate. For testing, mice received 0.5 ml of each mAb or 1 ml of two mAbs in combination (0.5 ml of each) intraperitoneally. Control mice received 0.5 ml of the DPBS diluents.

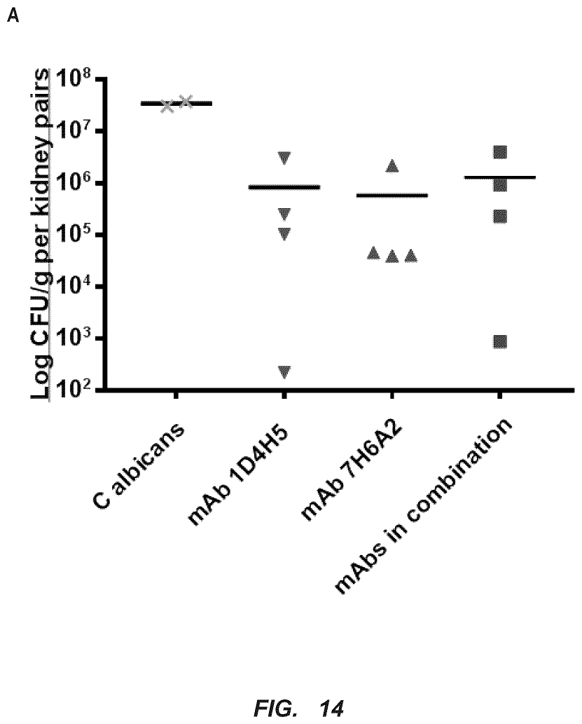

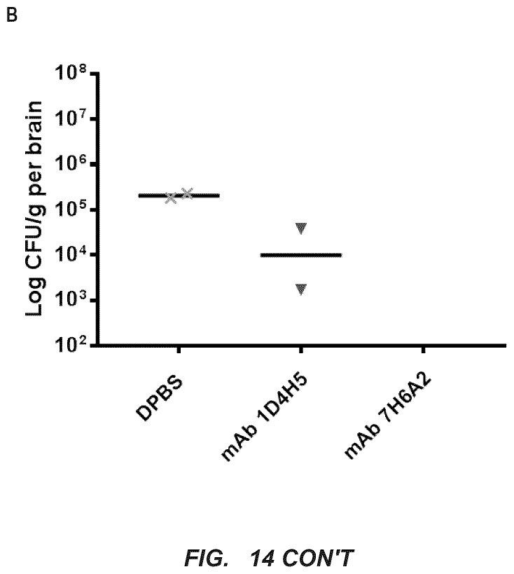

Protective mAbs protect immunocompromised mice against disseminated infection by Candida auris: The possibility of using passive mAb immunotherapy in immunocompromised mice against disseminated candidiasis by C. auris. The protective effect of mAb 7H6A2, 1D4H5 or these two mAbs in combination against the disease in immunocompromised mice was examined by passive transfer experiments (FIG. 14). Mice received either mAb 7H6A2 or 1D4H5 or the two mAbs in combination had reduced CFUs in kidneys (FIG. 14A) and in brains (FIG. 14B) as compared with control mice. Especially there is no CFU detected in brains of mice received mAb 7H6A2 treatment.

### Example 14

In embodiments, protective mAbs have enhanced/synergy efficacy when combined with conventional antimicrobial drugs against fungal infections, such as disseminated candidiasis caused by C. albicans in mice.

TABLE 16

| | MST (mean survival |
|---|---|
| Treatment | times) Days |
| None | 9.4 ± 2.1 |
| Amp B (0.5 mg/kg) | 14.5 ± 3.7 |
| mAb E2-9 + Amp B (0.5 mg/kg) | 29.4 ± 4.2 |
| mAb M2-4 + Amp B (0.5 mg/kg) | 37.4 ± 6.3 |
| mAb 2D5 + Amp B (0.5 mg/kg) | 39.1 ± 5.9 |
| mAb 2B10 + Amp B (0.5 mg/kg) | 46.7 ± 6.7 |

Table 16: mAbs have Enhanced/Synergy Efficacy when Combined with Conventional Antimicrobial Drugs Against Disseminated Candidiasis

Therapeutic effect of amphotericin B (Amp B) on mice with disseminated candidiasis is dose-dependent. BALB/c mice received minimal dose of Amp B (0.5 mg drug/kg body) appeared to be the same susceptible to the disease as mice that received no Amp B. Combination treatment of Amp B (0.5 mg/kg body weight) and each mAb (E2-9, M2-4, 2D5F7 and 2B10) at 8 hours post-infection significantly increased the survival time of mice receive the Amp B and mAb treatment, as compared to mice that received none treatment or with Amp B only.

### Materials and Methods

### Amp B—Antibody Combination Therapy

Prior to testing the effects of combination therapy with combination of Amp B & mAbs, the dose of the Amp B alone (0.5 mg/kg) that have no therapeutic effect on animals with disseminated candidiasis was determined. Mice were then given by i.v. of 5×10e5 of C. albicans 3153A yeast cells, 8 hour later the mice received i.p. 0.5 mg Amp B/kg body weight with each mAb (E2-9, M2-4, 2D5F7 and 2B10C1). Mice without any treatment and mice treated with antifungal drug alone were used as controls. Survival curves were scored and MST were measured.

### Example 14

TABLE 17

| SEQ | | |
|---|---|---|
| ID | Identifier | Sequence |
| 1 | GPV-M1 | FWVNPDCGLKTR |
| 2 | GPV-M2 | TTTIGSFPQTKDIR |
| 3 | GPV-M3 | ADKDSLDLEPISLLPK |
| 4 | GPV-M4 | YNLPLFPTTTIGSFPQTKDIR |
| 5 | GPV-M5 | DDVSGKIQALQLGLALR |
| 6 | GPV-M6 | GWPEVKESLTNMVEAAK |
| 7 | GPV-M7 | YTKFDLAPIDVLFAMGR |
| 8 | GPV-M8 | ASAVVQKAIEK |
| 9 | GPV-M9 | FWVNPDCGLK |
| 10 | GPV-M10 | FWVNPDCGLKTR |
| 11 | GPV-M11 | SESSITNDPKVQER |
| 12 | GPV-M12 | KFITDQTSIDLVADGR.T |
| 13 | GPV-M13 | KSTVASSDR |
| 14 | GPV-P1 | AILGGSKVSDKI |
| 15 | GPV-P2 | STGGGASLELLEGK |
| 16 | GPV-P3 | IVIIGGGDTATVAKK |
| 17 | GPV-P4 | ALENPERPFLAILGGAKVSDK.I |
| 18 | GPV-P5 | KVKADPEAVKK |
| 19 | GPV-P6 | LLGQKVTFLNDCVGPEVTKAVENAK |
| 20 | GPV-P7 | LSVKDLDVAGKR |
| 21 | GPV-P8 | MPIGDSLFDEAGAKNVEHLVEK |
| 22 | GPV-P9 | SAENGNIVIIGGGDTATVAKK |
| 23 | GPV-P10 | VTFLNDCVGPEVTK |
| 24 | GPV-P11 | NDKYSLAPVATELEK |
| 25 | GPV-P12 | YHIEEEGSSKDKDGK |
| 26 | Fba-C1 | IAEALDIFHTKGQL |
| 27 | Fba-C2 | EGADKPNKKYFDPRV |
| 28 | Fba-C3 | LKTIGGALQISDNSE |
| 29 | Fba | YGKDVKDLFDYAQE |
| 30 | Met6 | PRIGGQRELKKITE |
| 31 | Hwp1 | QGETEEALIQKRSY |
| 32 | Enol | DSRGNPTVEVDFTT |
| 33 | Gap1 | NRSPSTGEQKSSGI |
| 34 | Pgk1 | VPLDGKTITNNQRI |
| 35 | Fba 2 | FAIPAINVTSSSTV |
| 36 | Fba3/FbaU1 | FAIPAINVTSSSTVVAALE |
| 37 | Fba4 | SSSTVVAAL |
| 38 | Met6-2 | YDQVLDLSLLFNAIP |
| 39 | Fba Pep2 | Y GKDVKDLFDYAQE |
| 40 | Fba Pep3 | Y G KDVKDLFDYAQE |
| 41 | Fba Pep4 | YG K DVKDLFDYAQE |
| 42 | Fba Pep5 | YGKD V KDLFDYAQE |
| 43 | Fba Pep6 | YGKDV K DLFDYAQE |
| 44 | Fba Pep7 | YGKDVKD L FDYAQE |
| 45 | Fba Pep8 | YGKDVKDL F DYAQE |
| 46 | Fba Pep9 | YGKDVKDLFD Y AQE |
| 47 | Fba Pep10 | Y GKDVKD L FDYAQE |
| 48 | Fba Pep11 | YGKDCVKCDLFDYAQE |
| | linker | KK |
| 50 | linker | GPSL |
| 51 | PADRE | AKFVAAWTLKAAA |
| | ? | RGD |
| 53 | Fba | Accession No. AOW28947 |
| | | (Protein Accession No. XP_722690) |
| 54 | Met6 | Accession No. AOW30921 |
| | | (Protein Accession No. XP_718219 |
| 55 | Hwp1 | Accession No. ACN63125 |
| 56 | Enol | Accession No. AAA71939 |
| 57 | Gap1 | Accession No. AOW29704 |
| 58 | Pgk1 | Accession No. AAA66523 |
| 59 | 2B10C1 | heavy chain DNA |
| 60 | 2B10C1 | heavy chain amino acid |
| 61 | 2B10C1 | light chain DNA |
| 62 | 2B10C1 | light chain amino acid |
| 63 | 1D4H5 | heavy chain DNA |
| 64 | 1D4H5 | heavy chain amino acid |
| 65 | 1D4H5 | light chain DNA |
| 66 | 1D4H5 | light chain amino acid |
| 67 | 2D5F7 | heavy chain DNA |
| 68 | 2D5F7 | heavy chain amino acid |
| 69 | 2D5F7 | light chain DNA |
| 70 | 2D5F7 | light chain amino acid |
| 71 | 10E7E2 | heavy chain DNA |
| 72 | 10E7E2 | heavy chain amino acid |
| 73 | 10E7E2 | light chain DNA |
| 74 | 10E7E2 | light chain amino acid |
| 75 | TT 830-844 | QYIKANSKFIGITE |
| 76 | linker | EAAAK |
| 77 | linker | GGGGS |
| 78 | TT 946-967 | FNNFTVSFWLRVPKVSASHLE |
| 79 | GPV-MO2 | NPDCGLKTR |
| 80 | FbaU2 | PAINVTSSSTVVAALEAA |
| 81 | GPV-MO1 | PTTTIGSFPQ |
| 82 | GPV-MO1A | NLPLFPTTTIGSFPQTK |
| 83 | | PADRE-GPV-M1-GPSL-GPV-P1 |
| 84 | | TT830-844-GPSL-GPV-M1-GPSL-GPV-P1 |
| 85 | | TT947-967-GPSL-GPV-M1-GPSL-GPV-P1 |
| 86 | | RGD-GPV-P3-KK-GPV-M2 |
| 87 | | RGD-GPV-P3-KK-GPV-M2-KLH |
| 88 | | TT830-844-KK-GPV-P3-KK-GPV-M2 |
| 89 | | TT947-967-KK-GPV-P3-KK-GPV-M1 |
| 90 | | RGD-TT830-844-KK-GPV-P3-KK-GPV-M1 |
| 91 | | RGD-TT947-967-KK-GPV-P3-KK-GPV-M1 |
| 92 | | PADRE-GPV-M2-KK-GPV-P1 |
| 93 | | RGD-PADRE-GPV-M2-KK-GPV-P3 |

TABLE 18
Examples of hybridoma clones producing
protective mAbs, which are specific for
peptides derived from C. albicans cell
wall protein and the isotype of each
protective mAbs.
Hybridoma clone and related protective
peptide-specific mAbs

| | | mAb | peptide | peptide |
|---|---|---|---|---|
| Hybridomas | mAb | isotype | vaccine | Sequences |
| 2C9G3 | 2C9 | IgG2a | GPV-M2 | cTTTIGSFPQTKDIR |
| | | | universal | (SEQ ID NO: 2) |
| 5A9D1 | SA9 | IgG3 | GPV-M2 | cTTTIGSFPQTKDIR |
| | | | universal | (SEQ ID NO: 2) |
| 5A11B1 | SA1 | IgG3 | GPV-M2 | cTTTIGSFPQTKDIR |
| | | | universal | (SEQ ID NO: 2) |
| 10E7E2 | 10E7 | IgG1 | GPV-P3 | cIVIIGGGDTATVAKK |
| | | | universal | (SEQ ID NO: 16) |
| 2B10C1 | 2B10 | IgG1 | Met6 | PRIGGQRELKKITEc |
| | | | peptide | (SEQ ID NO: 30) |
| 7H6A2 | 7H6 | IgG2b | Met6 | PRIGGQRELKKITEc |
| | | | peptide | (SEQ ID NO: 30) |

TABLE 19
Examples of protective mAbs that have been tested in mouse model
(A/J strain) of disseminated candidiasis caused by C. auris.

| mAb | mAb isotype | against C. auris |
|---|---|---|
| 2C9 | IgG2a | High (80-100%) |
| 5A9 | IgG3 | High (80-90%) |
| 5A1 | IgG3 | High (60-90%) |
| 10E7 | IgG1 | High (80-100%) |
| 2B10 | IgG1 | High (60-90%) |
| 7H6 | IgG2b | High (80-100%) |

### Example 16

As shown herein, a panel of mAbs protect against C. auris invasive infection in newly established A/J mouse model of human disseminated candidiasis, including 6H1G8, 9F2G5, 10E7E2 and 3H7E3.

Also, mAbs in combination (such as, 9F2G5+3H7E3) provided enhanced protection as compared to 9F2G5 or 3H7E3 alone, such as in protection against C. auris invasive infection in mice.

Further, mAb 9F2G5 provides improved immunoprotection against C. auris disseminated candidiasis in mouse model as compared to anti-C. auris drug Micafungin, which is currently used in clinics.

Still further, 9F2G5 combined with Micafungin enhanced therapeutic potential of Micafungain, reducing CFUs in targeted organs as compared to Micafungin alone therapeutic treatment.

Thus, mAb 9F2G5 by itself not only protects against severe C. auris infection more effective than antifungal drug, but also functions to enhance conventional antimicrobial therapy of Micafungin.

### Introduction

Disseminated candidiasis is a life-threatening disease and a leading cause of bloodstream infections afflicting immunocompromised and hospitalized patients in the United States. Despite the availability of antifungal therapy, crude mortality in the last decade has remained unacceptably high (50-80%). The infection is caused by multiple species of the fungal genus Candida with C. albicans being the most common, together with C. tropicalis and C. glabrata, caused >90% disseminated candidiasis in humans. Of particular concern, “superbug” C. auris is a multi-drug resistant, health care-associated fungal pathogen, and has recently emerged as the first fungal pathogen to cause a global public health threat. Since there is no approved antifungal vaccine and current treatments are inadequate, antifungal antibodies could provide long-awaited new therapies for use alone or in combination with other agents, such as antifungal agents.

Shown herein are prophylactic/therapeutic antibodies that protect against disseminated candidiasis caused by C. auris. First, we identify peptide-specific mAbs protect against C. auris invasive infection in mouse model of human disseminated candidiasis. Secondly, different mAb combinations were further tested to identify synergistic efficacy of mAb cocktail. Finally, the mAb that has synergy/enhanced efficacy when combined with conventional antimicrobial drugs against disseminated candidiasis was identified.

### Supporting Data:

Evaluation and validation of a panel of peptide-specific monoclonal antibodies (mAbs) as a passive immunization strategy protect against invasive C. auris infection in established A/J mouse model of human disease.

The monoclonal antibodies used in this experiment were:
6H1G8 (IgG2b), α-Hwp1 (Hyphal wall protein)
3E2G2 (IgG2b), α-Fba (Fructose bisphosphate aldolase)
10E7E2 (IgG1), α-GPV-P3 (General peptide vaccine 3 from Phosphoglycerate kinase)
9F2G5 (IgG2a), α-Pgk1 (Phosphoglycerate kinase)
3H7E3 (IgG1), α-Met6 (Methyltetrahydropteroyltriglutamate)

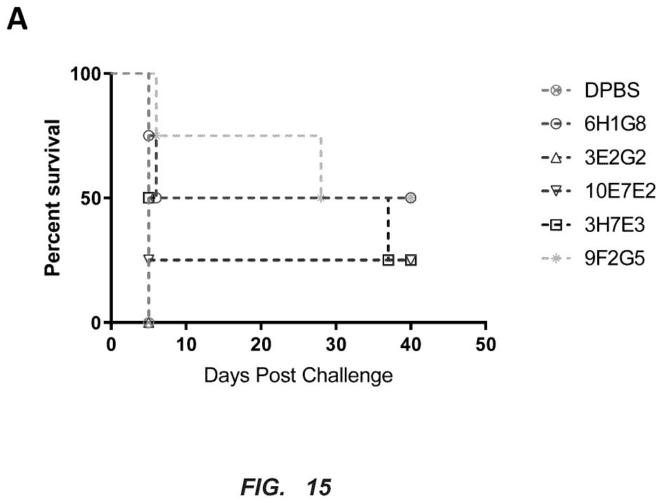

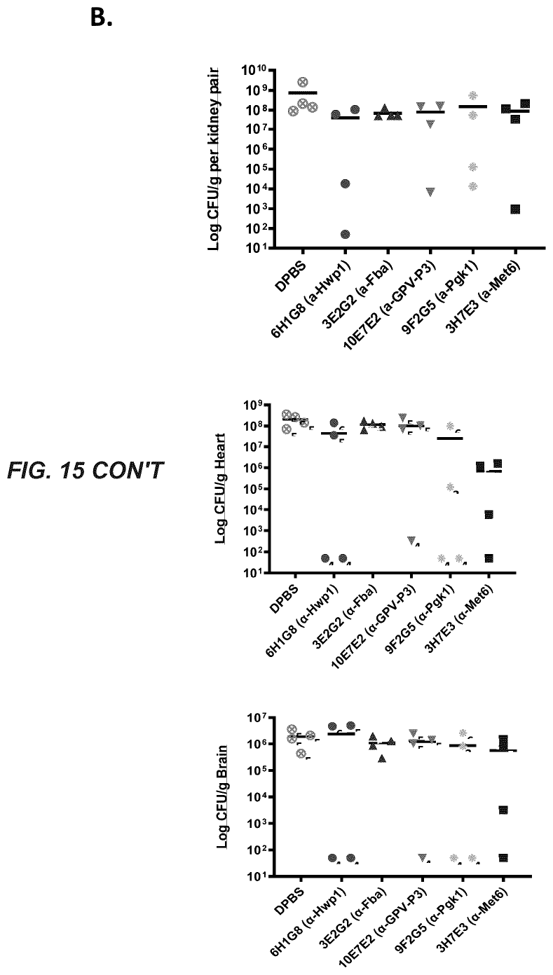

Referring to FIG. 15, for example, monoclonal antibodies (mAbs) are effective against invasive candidiasis caused by C. auris, the multidrug-resistant Candida species. For example, mAbs 6H1G8 and 9F2G5 provided best protection, evidenced by highest survival and lowest fungal burdens in targeted organs. mAbs 10E7E2 and 3H7E3 also provided some protection with 25% survival, and lower fungal loads in targeted organs.

Evaluation and Validation of Combination Therapy with mAb Cocktails in Established A/J Mouse Model of Disseminated Candidiasis.

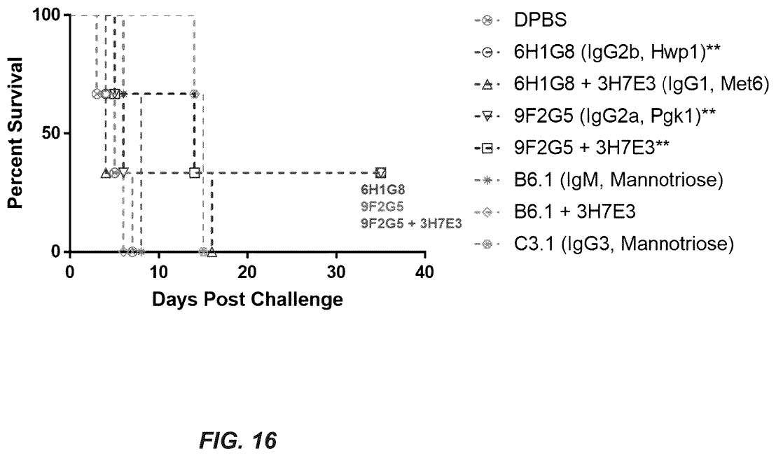

Referring to FIG. 16, for example, two protective mAbs in combination conferred best protection against systemic C. auris infection in passive transfer experiments. For example, two mAbs, such as 9F2G5+3H7E3 in combination, provided enhanced protection as compared to 9F2G5 or 3H7E3 alone. Mice that survived to Day 35 (6H1G8, 9F2G5, and 9F2G5+3H7E3) had lowest fungal burdens in all organs. Consistent with previous data, 6H1G8 and 9F2G5 were able to almost clear fungal burden (only one mouse per groups 6H1G8, 9F2G5, and 9F2G5+3H7E3 had fungal burdens lower than DPBS).

Validation of antifungal therapy of mAb in combination with antifungal agents. Herein we validate that mAb 9F2G5 is able to enhance the effectiveness of currently available antifungal drugs against C. auris. As an example, we evaluate micafungin, which is an effective drug against different strains of C. auris, and fluconazole as negative control, to which most strains of C. auris (including our own) is resistant.

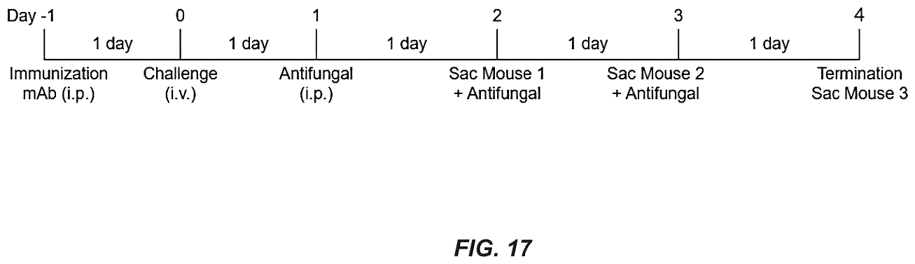

Method and Material to determine if mAb 9F2G5 (α-Pgk1, IgG2a) can increase antifungal efficacy in A/J mice. See FIG. 17, for example.

Micafungin (effective drug) Fluconazole (ineffective drug)

Experimental Groups (three mice/group):
1. DPBS
2. Micafungin (0.25 mg/kg/day)
3. Micafungin (0.5 mg/kg/day)
4. Micafungin (1.0 mg/kg/day)
5. 9F2G5
6. 9F2G5+Micafungin (0.25 mg/kg/day)
7. 9F2G5+Micafungin (0.5 mg/kg/day)
8. 9F2G5+Micafungin (1.0 mg/kg/day)
9. Fluconazole (25 mg/kg/day)
10. 9F2G5+Fluconazole (25 mg/kg/day)

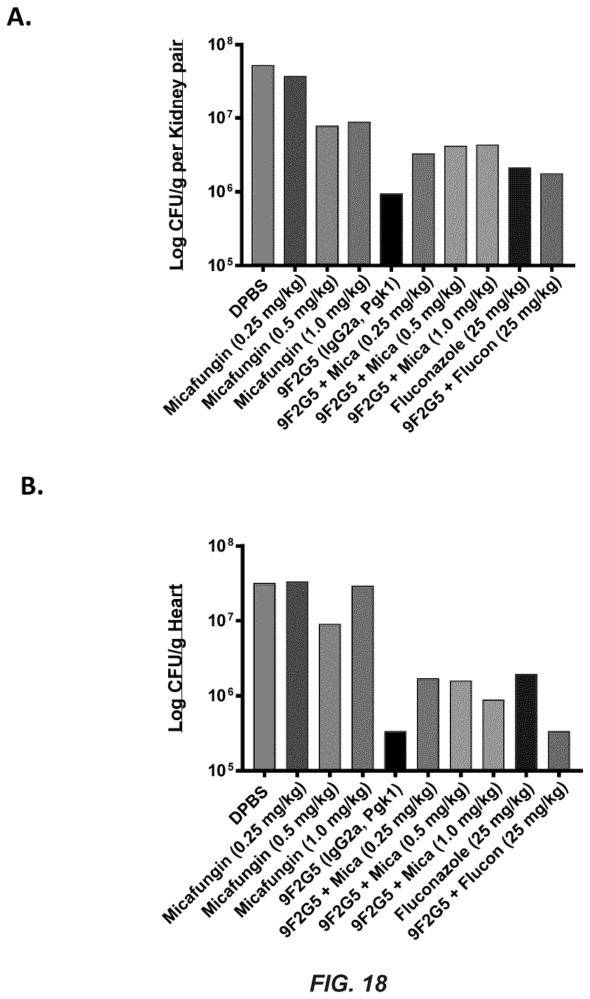

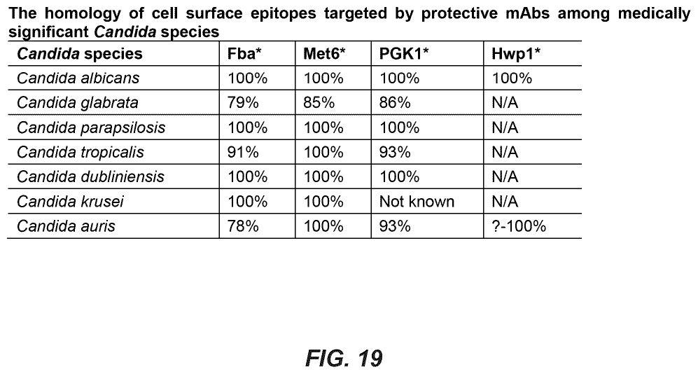

Referring to FIG. 18, mAb 9F2G5 used alone or with antifungal drug has therapeutic activity against C. auris disseminated candidiasis. Administration of 9F2G5 (α-Pgk1 peptide, IgG2a) alone provide better protection as compared to Micafungin alone therapeutic treatment, evidenced by reduced fungal burdens in both kidney (A) and heart (B) at day 4 post-infection. Antibody 9F2G5 given by i.p. 24 hours before challenge, combined with Micafungin given i.p. 24h post-infection had enhanced therapeutic potential of Micafungin, reducing CFUs in both kidney (A) and heart (B) significantly as compared to Micafungin alone therapeutic treatment.

Thus, mAb can function as an adjunct to existing antifungal therapy. In our study, mAb 9F2G5 used alone provided better protection as compared to antifungal agent Micafungin. Furthermore, mAb 9F2G5, combined with Micafungin, also enhanced therapeutic potential of Micafungin, reducing fungal loads in targeted organs as compared to Micafungin alone.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1-22. (canceled)

23. An isolated antibody or fragment thereof comprising:
a. 2B10C1 a VH CDR1 comprising the amino acid sequence of SYWMS (SEQ ID NO: 134), a VH CDR2 comprising the amino acid sequence of RIDPYDSETHYNQKFKD (SEQ ID NO: 135), a VH CDR3 comprising the amino acid sequence of TAASFDY (SEQ ID NO: 136), a VL CDR1 comprising the amino acid sequence of RSSQSLVHSNGNSYLH (SEQ ID NO: 158), a VL CDR2 comprising the amino acid sequence of KVSNRFS (SEQ ID NO: 159), and a VL CDR3 comprising the amino acid sequence of SQSTHVPFT (SEQ ID NO: 160); or
b. 1D4H5 a VH CDR1 comprising the amino acid sequence of NYWMN (SEQ ID NO: 137), a VH CDR2 comprising the amino acid sequence of EIRLKSNNYATHYAESVKG (SEQ ID NO: 138), a VH CDR3 comprising the amino acid sequence of GNY, a VL CDR1 comprising the amino acid sequence of SASSTISSNYLH (SEQ ID NO: 161), a VL CDR2 comprising the amino acid sequence of RTSNLAS (SEQ ID NO: 162), and a VL CDR3 comprising the amino acid sequence of QQGSTISRT (SEQ ID NO: 163); or
c. 2D5F7 a VH CDR1 comprising the amino acid sequence of SYTMH (SEQ ID NO: 140), a VH CDR2 comprising the amino acid sequence of YINPSSGYTDYNQKFKD (SEQ ID NO: 141), a VH CDR3 comprising the amino acid sequence of LYDNYDYYAMDY (SEQ ID NO: 142), a VL CDR1 comprising the amino acid sequence of KSSQSLLNSRIRKNYLA (SEQ ID NO: 164), a VL CDR2 comprising the amino acid sequence of WASTRES (SEQ ID NO: 165), and a VL CDR3 comprising the amino acid sequence of KQSYNLLT (SEQ ID NO: 166); or
d. 10E7E2 a VH CDR1 comprising the amino acid sequence of SYGVH (SEQ ID NO: 143), a VH CDR2 comprising the amino acid sequence of VIWSGGTTDYNAAFIS (SEQ ID NO: 144), a VH CDR3 comprising the amino acid sequence of GGHRGFAY (SEQ ID NO: 145), a VL CDR1 comprising the amino acid sequence of KASQDVGTAVA (SEQ ID NO: 167), a VL CDR2 comprising the amino acid sequence of WASTRHT (SEQ ID NO: 168), and a VL CDR3 comprising the amino acid sequence of QQYSSYPLT (SEQ ID NO: 169); or
e. 7C6E8-1 and 7C6E8-2 a VH CDR1 comprising the amino acid sequence of DYSMH (SEQ ID NO: 146), a VH CDR2 comprising the amino acid sequence of WINTETGEPTYADDFKG (SEQ ID NO: 147), a VH CDR3 comprising the amino acid sequence of NYYDTTGFAY (SEQ ID NO: 148), a VL CDR1 comprising the amino acid sequence of SEQ ID NO: KSSQRLLYSYGKTYLN (SEQ ID NO: 170), a VL CDR2 comprising the amino acid sequence of LVSKLDS (SEQ ID NO: 171), and a VL CDR3 comprising the amino acid sequence of VQDTHFPYT (SEQ ID NO: 172), or a VL CDR1 comprising the amino acid sequence of RASQSIGTSIH (SEQ ID NO: 173), a VL CDR2 comprising the amino acid sequence of FASESIS (SEQ ID NO: 174), and a VL CDR3 comprising the amino acid sequence of QQSNSWPTYT (SEQ ID NO: 175); or
f. 9F2G5 a VH CDR1 comprising the amino acid sequence of EYTMH (SEQ ID NO: 149), a VH CDR2 comprising the amino acid sequence of GINPNNGGTRYNQKFKG (SEQ ID NO: 150), a VH CDR3 comprising the amino acid sequence of YFPY (SEQ ID NO: 151), a VL CDR1 comprising the amino acid sequence of RSSQSLLDSDGKTYLN (SEQ ID NO: 176), a VL CDR2 comprising the amino acid sequence of LVSKLDS (SEQ ID NO: 177), and a VL CDR3 comprising the amino acid sequence of WQGTHFPWT (SEQ ID NO: 178); or,
g. 6H1G8 a VH CDR1 comprising the amino acid sequence of GYYMH (SEQ ID NO: 152), a VH CDR2 comprising the amino acid sequence of RINPYTGATSYTQNFKD (SEQ ID NO: 153), a VH CDR3 comprising the amino acid sequence of GGGRSSYWYFDV (SEQ ID NO: 154), a VL CDR1 comprising the amino acid sequence of SASQAISNYLN (SEQ ID NO: 179), a VL CDR2 comprising the amino acid sequence of YTSSLHS (SEQ ID NO: 180), and a VL CDR3 comprising the amino acid sequence of QQYSKLPWT (SEQ ID NO: 181); or
h. 7H6A2 a VH CDR1 comprising the amino acid sequence of DFSMH (SEQ ID NO: 155), a VH CDR2 comprising the amino acid sequence of WINTETVEPTYADDFKG (SEQ ID NO: 156), a VH CDR3 comprising the amino acid sequence of VSYDGYYGDFAMDY (SEQ ID NO: 157), a VL CDR1 comprising the amino acid sequence of RASQDISNYLN (SEQ ID NO: 182), a VL CDR2 comprising the amino acid sequence of YTSRLHS (SEQ ID NO: 183), and a VL CDR3 comprising the amino acid sequence of QQGNTLPWT (SEQ ID NO: 184).

24. The antibody or fragment of claim 23, wherein the antibody is fully human or humanized.

25. The antibody or fragment of claim 23, wherein the antibody is an IgG.

26. The antibody or fragment of claim 23, wherein the antibody is a single chain antibody or a single chain fragment.

27. The antibody or fragment of claim 23, wherein the fragment comprises an Fᵥ fragment, a Fab fragment, a F(ab′) fragment, a F(ab′)₂ fragment, a disulfide stabilized Fᵥ protein (dsFv) fragment, a scFv fragment, a minibody fragment, or a diabody fragment.

28. The antibody or fragment of claim 23, further comprising a heavy chain constant region, a light chain constant region, an Fc region, or a combination thereof.

29. The antibody or fragment of claim 23, wherein the antibody comprises 2B10C1, 1D4H5, 2D5F7, 10E7E2, 7C6E8, 9F2G5, 6H1G8, or 7H6A2.

30. The antibody or fragment of claim 23, wherein the antibody competes with binding of 2B10C1, 1D4H5, 2D5F7, 10E7E2, 7C6E8, 9F2G5, 6H1G8, or 7H6A2.

31. The antibody or fragment of claim 23, wherein the antibody or fragment is linked to a therapeutic agent.

32. The antibody or fragment of claim 23, wherein the antibody or fragment comprises a heavy chain variable region and a light chain variable region, wherein
a. the heavy chain variable region comprises an amino acid sequence about 95% identical to SEQ ID NO: 60, and the light chain variable region comprises an amino acid sequence about 95% identical to SEQ ID NO: 62; or
b. the heavy chain variable region comprises an amino acid sequence about 95% identical to SEQ ID NO: 64, and the light chain variable region comprises an amino acid sequence about 95% identical to SEQ ID NO: 66; or
c. the heavy chain variable region comprises an amino acid sequence about 95% identical to SEQ ID NO: 68, and the light chain variable region comprises an amino acid sequence about 95% identical to SEQ ID NO: 70; or
d. the heavy chain variable region comprises an amino acid sequence about 95% identical to SEQ ID NO: 72, and the light chain variable region comprises an amino acid sequence about 95% identical to SEQ ID NO: 74; or
e. the heavy chain variable region comprises an amino acid sequence about 95% identical to SEQ ID NO: 108, and the light chain variable region comprises an amino acid sequence about 95% identical to SEQ ID NO: 109 or SEQ ID NO: 110; or
f. the heavy chain variable region comprises an amino acid sequence about 95% identical to SEQ ID NO: 111, and the light chain variable region comprises an amino acid sequence about 95% identical to SEQ ID NO: 112; or
g. the heavy chain variable region comprises an amino acid sequence about 95% identical to SEQ ID NO: 113, and the light chain variable region comprises an amino acid sequence about 95% identical to SEQ ID NO: 114; or
h. the heavy chain variable region comprises an amino acid sequence about 95% identical to SEQ ID NO: 114, and the light chain variable region comprises an amino acid sequence about 95% identical to SEQ ID NO: 116.

33. A nucleic acid encoding the antibody or fragment according to claim 23.

34. A vector comprising the nucleic acid of claim 33.

35. A cell comprising the vector of claim 34.

36. A pharmaceutical composition comprising the antibody or fragment according to claim 23, and a pharmaceutically acceptable carrier or excipient.

37. A method of treating or preventing a microbial infection, the method comprising administering to the subject the antibody or fragment according to claim 23.

38. The method of claim 37, wherein the microbial infection comprises a fungal infection, a bacterial infection, a viral infection, or a protozoal infection.

39. The method of claim 37, further comprising administering at least one antimicrobial agent.

40. The method of claim 39, wherein the antimicrobial agent comprises an anti-fungal agent, an anti-viral agent, an anti-bacterial agent, or an anti-protozoal agent.

41. The method of claim 38, wherein the fungal infection comprises Candida spp.


SEQUENCE LISTING (274 sequences)

SEQ ID NO: 1 - protein, 13 aa, unidentified
  VARIANT 1: note = Residue may be deleted
  source 1..13: note = GPV-M1
CFWVNPDCGLKTR

SEQ ID NO: 2 - protein, 15 aa, unidentified
  VARIANT 1: note = Residue may be deleted
  source 1..15: note = GPV-M2
CTTTIGSFPQTKDIR

SEQ ID NO: 3 - protein, 16 aa, unidentified
  source 1..16: note = GPV-M3
ADKDSLDLEPISLLPK

SEQ ID NO: 4 - protein, 21 aa, unidentified
  source 1..21: note = GPV-M4
YNLPLFPTTTIGSFPQTKDIR

SEQ ID NO: 5 - protein, 17 aa, unidentified
  source 1..17: note = GPV-M5
DDVSGKIQALQLGLALR

SEQ ID NO: 6 - protein, 17 aa, unidentified
  source 1..17: note = GPV-M6
GWPEVKESLTNMVEAAK

SEQ ID NO: 7 - protein, 17 aa, unidentified
  source 1..17: note = GPV-M7
YTKFDLAPIDVLFAMGR

SEQ ID NO: 8 - protein, 11 aa, synthetic construct
ASAVVQKAIEK

SEQ ID NO: 9 - protein, 10 aa, synthetic construct
FWVNPDCGLK

SEQ ID NO: 10 - protein, 12 aa, synthetic construct
FWVNPDCGLKTR

SEQ ID NO: 11 - protein, 14 aa, synthetic construct
SESSITNDPKVQER

SEQ ID NO: 12 - protein, 17 aa, synthetic construct
KFITDQTSIDLVADGRT

SEQ ID NO: 13 - protein, 9 aa, synthetic construct
KSTVASSDR

SEQ ID NO: 14 - protein, 13 aa, unidentified
  VARIANT 1: note = Residue may be deleted
  source 1..13: note = GPV-P1
CAILGGSKVSDKI

SEQ ID NO: 15 - protein, 14 aa, unidentified
  source 1..14: note = GPV-P2
STGGGASLELLEGK

SEQ ID NO: 16 - protein, 16 aa, unidentified
  VARIANT 1: note = Residue may be deleted
  source 1..16: note = GPV-P3
CIVIIGGGDTATVAKK

SEQ ID NO: 17 - protein, 22 aa, synthetic construct
ALENPERPFLAILGGAKVSDKI

SEQ ID NO: 18 - protein, 11 aa, synthetic construct
KVKADPEAVKK

SEQ ID NO: 19 - protein, 25 aa, synthetic construct
LLGQKVTFLNDCVGPEVTKAVENAK

SEQ ID NO: 20 - protein, 12 aa, synthetic construct
LSVKDLDVAGKR

SEQ ID NO: 21 - protein, 22 aa, synthetic construct
MPIGDSLFDEAGAKNVEHLVEK

SEQ ID NO: 22 - protein, 21 aa, synthetic construct
SAENGNIVIIGGGDTATVAKK

SEQ ID NO: 23 - protein, 14 aa, synthetic construct
VTFLNDCVGPEVTK

SEQ ID NO: 24 - protein, 15 aa, synthetic construct
NDKYSLAPVATELEK

SEQ ID NO: 25 - protein, 15 aa, synthetic construct
YHIEEEGSSKDKDGK

SEQ ID NO: 26 - protein, 14 aa, synthetic construct
IAEALDIFHTKGQL

SEQ ID NO: 27 - protein, 15 aa, synthetic construct
EGADKPNKKYFDPRV

SEQ ID NO: 28 - protein, 15 aa, synthetic construct
LKTIGGALQISDNSE

SEQ ID NO: 29 - protein, 15 aa, synthetic construct
  VARIANT 15: note = Residue may be deleted
YGKDVKDLFDYAQEC

SEQ ID NO: 30 - protein, 15 aa, synthetic construct
  VARIANT 15: note = Residue may be deleted
PRIGGQRELKKITEC

SEQ ID NO: 31 - protein, 15 aa, synthetic construct
  VARIANT 15: note = Residue may be deleted
QGETEEALIQKRSYC

SEQ ID NO: 32 - protein, 14 aa, synthetic construct
DSRGNPTVEVDFTT

SEQ ID NO: 33 - protein, 14 aa, synthetic construct
NRSPSTGEQKSSGI

SEQ ID NO: 34 - protein, 14 aa, synthetic construct
VPLDGKTITNNQRI

SEQ ID NO: 35 - protein, 14 aa, synthetic construct
FAIPAINVTSSSTV

SEQ ID NO: 36 - protein, 19 aa, synthetic construct
FAIPAINVTSSSTVVAALE

SEQ ID NO: 37 - protein, 9 aa, synthetic construct
SSSTVVAAL

SEQ ID NO: 38 - protein, 15 aa, synthetic construct
YDQVLDLSLLFNAIP

SEQ ID NO: 39 - protein, 14 aa, synthetic construct
YGKDVKDLFDYAQE

SEQ ID NO: 40 - protein, 14 aa, synthetic construct
YGKDVKDLFDYAQE

SEQ ID NO: 41 - protein, 14 aa, synthetic construct
YGKDVKDLFDYAQE

SEQ ID NO: 42 - protein, 14 aa, synthetic construct
YGKDVKDLFDYAQE

SEQ ID NO: 43 - protein, 14 aa, synthetic construct
YGKDVKDLFDYAQE

SEQ ID NO: 44 - protein, 14 aa, synthetic construct
YGKDVKDLFDYAQE

SEQ ID NO: 45 - protein, 14 aa, synthetic construct
YGKDVKDLFDYAQE

SEQ ID NO: 46 - protein, 14 aa, synthetic construct
YGKDVKDLFDYAQE

SEQ ID NO: 47 - protein, 14 aa, synthetic construct
YGKDVKDLFDYAQE

SEQ ID NO: 48 - protein, 16 aa, synthetic construct
YGKDCVKCDLFDYAQE

SEQ ID NO: 49 - skipped

SEQ ID NO: 50 - protein, 4 aa, synthetic construct
GPSL

SEQ ID NO: 51 - protein, 13 aa, synthetic construct
AKFVAAWTLKAAA

SEQ ID NO: 52 - skipped

SEQ ID NO: 53 - protein, 359 aa, Candida albicans
MAPPAVLSKSGVIYGKDVKDLFDYAQEKGFAIPAINVTSSSTVVAALEAARDNKAPIILQTSQGGAAYFAGKGVDNKDQAASIAGSIAAAHYIRAIAPTYGIPVVLHTDHCAKKLLPWFDGMLKADEEFFAKTGTPLFSSHMLDLSEETDDENIATCAKYFERMAKMGQWLEMEIGITGGEEDGVNNEHVEKDALYTSPETVFAVYESLHKISPNFSIAAAFGNVHGVYKPGNVQLRPEILGDHQVYAKKQIGTDAKHPLYLVFHGGSGSTQEEFNTAIKNGVVKVNLDTDCQYAYLTGIRDYVTNKIEYLKAPVGNPEGADKPNKKYFDPRVWVREGEKTMSKRIAEALDIFHTKGQL

SEQ ID NO: 54 - protein, 767 aa, Candida albicans
MVQSSVLGFPRIGGQRELKKITEAYWSGKATVEELLAKGKELREHNWKLQQKAGVDIIPSNDFSYYDQVLDLSLLFNAIPERYTKFDLAPIDVLFAMGRGLQKKATETQAAVDVTALEMVKWFDSNYHYVRPTFSHSTEFKLNTAAGIKPVDEFNEAKALGVQTRPVILGPVSYLYLGKADKDSLDLEPISLLPKILPVYKELLQKLKEAGAEQVQIDEPVLVLDLPEAVQSKFKEAYDALVGADVPELILTTYFGDVRPNLKAIENLPVAGFHFDFVRVPEQLDEVASILKDGQTLSAGVVDGRNIWKTDFAKASAVVQKAIEKVGKDKVVVATSSSLLHTPVDLESETKLDAVIKDWFSFATQKLDEVVVIAKNVSGEDVSKQLEANAASIKARSESSITNDPKVQERLTTINEALATRKAAFPERLTEQKAKYNLPLFPTTTIGSFPQTKDIRINRNKFAKGQITAEEYEAFINKEIETVVRFQEEIGLDVLVHGEPERNDMVQYFGEQLNGFAFTTNGWVQSYGSRYVRPPIIVGDVSRPKAMTVKESVYAQSITSKPMKGMLTGPVTILRWSFPRDDVSGKIQALQLGLALRDEVNDLEGAGITVIQVDEPAIREGLPLRAGKERSDYLNWAAQSFRVATSGVENSTQIHSHFCYSDLDPNHIKALDADVVSIEFSKKDDPNYIQEFSEYPNHIGLGLFDIHSPRIPSKQEFVSRIEEILKVYPASKFWVNPDCGLKTRGWPEVKESLTNMVEAAKEFRAKY

SEQ ID NO: 55 - protein, 264 aa, Candida albicans
SSSAPATTPNTSVPTTTTETKSSSTPLTTTTEHDTTVVTVTSCSNSVCTESEVTTGVIVITSKDTIYTTYCPLTETTPVSTAPATETPTGTVSTSTEQSTTVITVTSCSESSCTESEVTTGVVVVTSEETVYTTFCPLTENTPGTDSTPEASIPPMETIPAGSQPSIPAGETSPAVPKSDVPATESAPAPEMTPAGTETKPAAPKSSAPATEPSPVAPGTESAPAGPGASSSPKSSVLASETSPIAPGAETAPAGSSGAITIPE

SEQ ID NO: 56 - protein, 440 aa, Candida albicans [sequence omitted]

SEQ ID NO: 57 - protein, 582 aa, Candida albicans
MLHKKETNDTFVQLNRSPSTGEQKSSGIWSSIKDSFKPALPQDKLTGVDDIPDRELTDIERININAANSNLQRKLKTRHLQMIAIGSSIGTGLFVGTGGALSTGGPAAIVLAWAISAISVFMTMQGLGELAVAFPVSGGFNLYASKFLEPGIGFAVGWNYFLQFFVLLPLELVAGAITIKYWNASINSDVFVIIFWFVVLVITMLGVRWYGEAELVFCTIKVIAVIGFIILGIVLICGGGPNHEFIGGKYWREPGPFANSFKGFASSLITAAFSFGGTEMIALTASESSNVRHALPKAIKQVFWRIVIFYLGSIIMIATLVPYNDKRLLGSSSVDVTASPFTIAIVNGGIKGLPSVINAVILISVLSVGNASVYATSRTLNSLAEQGMAPKWTGYIDRAGRPLFAILITNVFGLFALIAADNEKQVVAFNWLLALSGLSSIFTWMSINLSHIRFRRAMKVQNRSLTELPFVAQSGVWGSYFGLTLNILYLIAQFYIGLFPVGGKPNAYDFFLAYLGVPVILASWIGYKIWKRDWTLFIRAKDIDLDTGRINVDLDLLQQEIAEEKAQLAEKPFYIRIYRFWC

SEQ ID NO: 58 - protein, 417 aa, Candida albicans
MSLSNKLSVKDLDVAGKRVFIRVDFNVPLDGKTITNNQRIVAALPTIKYVEEHKPKYIVLASHLGRPNGERNDKYSLAPVATELEKLLGQKVTFLNDCVGPEVTKAVENAKDGEIFLLENLRYHIEEEGSSKDKDGKKVKADPEAVKKFRQELTSLADVYINDAFGTAHRAHSSMVGLEVPQRAAGFLMSKELEYFAKALENPERPFLAILGGAKVSDKIQLIDNLLDKVDMLIVGGGMAFTFKKILNKMPIGDSLFDEAGAKNVEHLVEKAKKNNVELILPVDFVTADKFDKDAKTSSATDAEGIPDNWMGLDCGPKSVELFQQAVAKAKTIVWNGPPGVFEFEKFANGTKSLLDAAVKSAENGNIVIIGGGDTATVAKKYGVVEKLSHVSTGGGASLELLEGKDLPGVVALSNKN

SEQ ID NO: 59 - DNA, 405 bp, synthetic construct
  source 1..405: mol_type = other DNA
atgggatggagctatatcatcctcttcttgttagcaacagctacacgtgtccactcccaggtccaactgcagcagcctggggctgaggtggtgaggcctggggcttcagtgaaggtgtcctgcaaggcttctggctacacggtcagcagctactggatgagctgggttaagcagaggccggagcaaggccttgagtggattggaaggattgatccttacgatagtgaaactcactacaatcaaaagttcaaggacaaggccatattgactgtagacaaatcctccagcacagcctacatgcaactcagcagcctgacatctgaggactctgcggtctattactgtgcaaggacggccgcttcgtttgactattggggccaaggcaccactctcacagtctcctca

SEQ ID NO: 60 - protein, 135 aa, synthetic construct
MGWSYIILFLLATATRVHSQVQLQQPGAEVVRPGASVKVSCKASGYTVSSYWMSWVKQRPEQGLEWIGRIDPYDSETHYNQKFKDKAILTVDKSSSTAYMQLSSLTSEDSAVYYCARTAASFDYWGQGTTLTVSS

SEQ ID NO: 61 - DNA, 393 bp, synthetic construct
  source 1..393: mol_type = other DNA
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaactcctatttacattggtacctgcagaagccaggccagtctccaaagctcctgatctacaaagtttccaaccgattttctggggtcccagacaggttcagtggcagtggatcagggacagatttcacactcaatatcagcagagtggaggctgaggatctgggagtttatttctgctctcaaagtacacatgttccattcacgttcggctcggggacaaagttggaaataaaa

SEQ ID NO: 62 - protein, 131 aa, synthetic construct
MKLPVRLLVLMFWIPASSSDVVMTQTPLSLPVSLGDQASISCRSSQSLVHSNGNSYLHWYLQKPGQSPKLLIYKVSNRFSGVPDRFSGSGSGTDFTLNISRVEAEDLGVYFCSQSTHVPFTFGSGTKLEIK

SEQ ID NO: 63 - DNA, 399 bp, synthetic construct
  source 1..399: mol_type = other DNA
atgtacttgggactgaactgtgtattcatagtttttctcttaaaaggtgtccagagtgaagtgaagcttgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagcgatgattccaaaagtagtgtctacctgcaaatgaacaacttaagagctgaagacactggcatttattactgttcaactgggaactactggggccaaggcaccactctcacagtctcctca

SEQ ID NO: 64 - protein, 133 aa, synthetic construct
MYLGLNCVFIVFLLKGVQSEVKLEESGGGLVQPGGSMKLSCVASGFTFSNYWMNWVRQSPEKGLEWVAEIRLKSNNYATHYAESVKGRFTISSDDSKSSVYLQMNNLRAEDTGIYYCSTGNYWGQGTTLTVSS

SEQ ID NO: 65 - DNA, 390 bp, synthetic construct
  source 1..390: mol_type = other DNA
atggattttcagatgcagattatcagcttgctgctaatcagtgtcacagtcatagtgtctaatggagaaattgtgctcacccagtctccaaccaccatggctgcatctcccggggagaagatcactatcacctgcagtgccagctcaactataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtactatatcacgcacgttcggctcggggacaaagttggaaataaaa

SEQ ID NO: 66 - protein, 130 aa, synthetic construct
MDFQMQIISLLLISVTVIVSNGEIVLTQSPTTMAASPGEKITITCSASSTISSNYLHWYQQKPGFSPKLLIYRTSNLASGVPARFSGSGSGTSYSLTIGTMEAEDVATYYCQQGSTISRTFGSGTKLEIK

SEQ ID NO: 67 - DNA, 420 bp, synthetic construct
  source 1..420: mol_type = other DNA
atggaaaggcactggatctttctcttcctgttgtcagtaactgcaggtgtccactcccaggtccagctgcagcagtctgcagctgaactggcaagacctggggcctcagtgaagatgtcctgcaaggcttctggctacacctttagtagctacacgatgcactgggtaaaacagaggcctggacagggtctggaatggattggatacattaatcctagcagtggatatactgattacaatcagaagttcaaggacaagaccacattgactgcagacaaatcctccagcacagcctacatgcaactgagcagcctgacatctgaggactctgcggtctattactgtgcaagactatatgataactacgattactatgctatggactactggggtcaaggaacctcagtcaccgtctcctca

SEQ ID NO: 68 - protein, 140 aa, synthetic construct
MERHWIFLFLLSVTAGVHSQVQLQQSAAELARPGASVKMSCKASGYTFSSYTMHWVKQRPGQGLEWIGYINPSSGYTDYNQKFKDKTTLTADKSSSTAYMQLSSLTSEDSAVYYCARLYDNYDYYAMDYWGQGTSVTVSS

SEQ ID NO: 69 - DNA, 396 bp, synthetic construct
  source 1..396: mol_type = other DNA
atggattcacaggcccaggttcttatattgctgctgctatgggtatctggtacctgtggggacattgtgatgtcacagtctccatcctccctggctgtgtcagcaggagagaaggtcactatgagctgcaaatccagtcagagtctgctcaatagtagaatccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgatgacctggcagtttattactgcaagcaatcttataatctgctcacgttcggtgctgggaccaagctggagctgaaa

SEQ ID NO: 70 - protein, 132 aa, synthetic construct
MDSQAQVLILLLLWVSGTCGDIVMSQSPSSLAVSAGEKVTMSCKSSQSLLNSRIRKNYLAWYQQKPGQSPKLLIYWASTRESGVPDRFTGSGSGTDFTLTISSVQADDLAVYYCKQSYNLLTFGAGTKLELK

SEQ ID NO: 71 - DNA, 405 bp, synthetic construct
  source 1..405: mol_type = other DNA
atggctgtcttggggctgctcttctgcctggtgacattcccaagctgtgtcccatcccaggtgcagctgaagcagtcaggacctggcctagtgcagccctcacagagcctgtccatcacctgcacagtctctggtttctcattaactagctatggtgtacactgggttcgccagtctccaggaaagggtctggagtggctgggagtgatatggagtggtggaactacagactataatgcagctttcatatccagactgagcatcagcaaggacaattccaagagccaagttttctttaaaatgaacagtctgcaagctaatgacacagccatatattactgtgccagaggggggcaccgagggtttgcttactggggccaagggactctggtcactgtctctgca

SEQ ID NO: 72 - protein, 135 aa, synthetic construct
MAVLGLLFCLVTFPSCVPSQVQLKQSGPGLVQPSQSLSITCTVSGFSLTSYGVHWVRQSPGKGLEWLGVIWSGGTTDYNAAFISRLSISKDNSKSQVFFKMNSLQANDTAIYYCARGGHRGFAYWGQGTLVTVSA

SEQ ID NO: 73 - DNA, 393 bp, synthetic construct
  source 1..393: mol_type = other DNA
atgggcatcaagatggagacacattctcaggtctttgtatacatgttgctgtggttgtctggtgttgaaggagacattgtgatgacccagtctcacaaattcatgtccacatcagtaggagacagggtcagcatcacctgcaaggccagtcaagatgtgggtactgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcctctcacgttcggtgctgggaccaagctggagctgaaa

SEQ ID NO: 74 - protein, 131 aa, synthetic construct
MGIKMETHSQVFVYMLLWLSGVEGDIVMTQSHKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLELK

SEQ ID NO: 75 - protein, 14 aa, unidentified
  source 1..14: note = universal T-cell epitope
QYIKANSKFIGITE

SEQ ID NO: 76 - protein, 5 aa, synthetic construct
EAAAK

SEQ ID NO: 77 - protein, 5 aa, synthetic construct
GGGGS

SEQ ID NO: 78 - protein, 21 aa, unidentified
  source 1..21: note = universal T-cell epitope
FNNFTVSFWLRVPKVSASHLE

SEQ ID NO: 79 - protein, 9 aa, unidentified
  source 1..9: note = GPV-MO2
NPDCGLKTR

SEQ ID NO: 80 - protein, 18 aa, synthetic construct
PAINVTSSSTVVAALEAA

SEQ ID NO: 81 - protein, 10 aa, unidentified
  source 1..10: note = GPV-MO1
PTTTIGSFPQ

SEQ ID NO: 82 - protein, 17 aa, unidentified
  source 1..17: note = GPV-MO1A
NLPLFPTTTIGSFPQTK

SEQ ID NO: 83 - protein, 43 aa, synthetic construct
  VARIANT 14: note = Residue may be deleted
  VARIANT 31: note = Residue may be deleted
AKFVAAWTLKAAACFWVNPDCGLKTRGPSLCAILGGSKVSDKI

SEQ ID NO: 84 - protein, 48 aa, synthetic construct
  VARIANT 19: note = Residue may be deleted
  VARIANT 36: note = Residue may be deleted
QYIKANSKFIGITEGPSLCFWVNPDCGLKTRGPSLCAILGGSKVSDKI

SEQ ID NO: 85 - protein, 55 aa, synthetic construct
  VARIANT 26: note = Residue may be deleted
  VARIANT 43: note = Residue may be deleted
FNNFTVSFWLRVPKVSASHLEGPSLCFWVNPDCGLKTRGPSLCAILGGSKVSDKI

SEQ ID NO: 86 - protein, 36 aa, synthetic construct
  VARIANT 4: note = Residue may be deleted
  VARIANT 22: note = Residue may be deleted
RGDCIVIIGGGDTATVAKKKKCTTTIGSFPQTKDIR

SEQ ID NO: 87 - protein, 39 aa, synthetic construct
  VARIANT 4: note = Residue may be deleted
  VARIANT 22: note = Residue may be deleted
RGDCIVIIGGGDTATVAKKKKCTTTIGSFPQTKDIRKLH

SEQ ID NO: 88 - protein, 49 aa, synthetic construct
  VARIANT 17: note = Residue may be deleted
  VARIANT 35: note = Residue may be deleted
QYIKANSKFIGITEKKCIVIIGGGDTATVAKKKKCTTTIGSFPQTKDIR

SEQ ID NO: 89 - protein, 54 aa, synthetic construct
  VARIANT 24: note = Residue may be deleted
  VARIANT 42: note = Residue may be deleted
FNNFTVSFWLRVPKVSASHLEKKCIVIIGGGDTATVAKKKKCFWVNPDCGLKTR

SEQ ID NO: 90 - protein, 50 aa, synthetic construct
  VARIANT 20: note = Residue may be deleted
  VARIANT 38: note = Residue may be deleted
RGDQYIKANSKFIGITEKKCIVIIGGGDTATVAKKKKCFWVNPDCGLKTR

SEQ ID NO: 91 - protein, 57 aa, synthetic construct
  VARIANT 27: note = Residue may be deleted
  VARIANT 45: note = Residue may be deleted
RGDFNNFTVSFWLRVPKVSASHLEKKCIVIIGGGDTATVAKKKKCFWVNPDCGLKTR

SEQ ID NO: 92 - protein, 43 aa, synthetic construct
  VARIANT 14: note = Residue may be deleted
  VARIANT 31: note = Residue may be deleted
AKFVAAWTLKAAACTTTIGSFPQTKDIRKKCAILGGSKVSDKI

SEQ ID NO: 93 - protein, 49 aa, synthetic construct
  VARIANT 17: note = Residue may be deleted
  VARIANT 34: note = Residue may be deleted
RGDAKFVAAWTLKAAACTTTIGSFPQTKDIRKKCIVIIGGGDTATVAKK

SEQ ID NO: 94 - protein, 6 aa, synthetic construct
HHHHHH

SEQ ID NO: 95 - protein, 14 aa, Candida albicans
YGKDVKDLFDYAQE

SEQ ID NO: 96 - protein, 14 aa, Candida albicans
PRIGGQRELKKITE

SEQ ID NO: 97 - protein, 15 aa, synthetic construct
  SITE 1: note = D-Alanine
  SITE 3: note = l-cyclohexylalanine
  SITE 13: note = D-Alanine
  SITE 14: note = Aminocaproic acid
AKXVAAWTLKAAAXC

SEQ ID NO: 98-107 - skipped (10 entries)

SEQ ID NO: 108 - protein, 119 aa, synthetic construct
QIQLVQSGPELKKPGETVKISCKASGYTFTDYSMHWVKQTPGKGLKWMGWINTETGEPTYADDFKGRFAFSLETSASTAYLQINNLKNEDTATYFCARNYYDTTGFAYWGQGTLVTVSA

SEQ ID NO: 109 - protein, 112 aa, synthetic construct
DVVMTQTPLTLSVTVGQPASISCKSSQRLLYSYGKTYLNWLLQRPGQSPKRLIYLVSKLDSGVPDRFTGSGSGTDFTLKISRVEAEDLGVYYCVQDTHFPYTFGGGTKLEIK

SEQ ID NO: 110 - protein, 108 aa, synthetic construct
DILLTQSPAILSVSPGERVSFSCRASQSIGTSIHWYQQRTNGSPRLLIKFASESISGIPSRFSGSGSGTDFPLSINSVESEDIAGYYCQQSNSWPTYTFGGGTKLEIK

SEQ ID NO: 111 - protein, 113 aa, synthetic construct
EVHLQQSGPELVKPGASVKISCKTSGYIFTEYTMHWVKQSHGKSLEWIGGINPNNGGTRYNQKFKGKATLTVDKSSSTAYMELRSLTSEDSAVYYCAWYFPYWGQGTLVTVSA

SEQ ID NO: 112 - protein, 112 aa, synthetic construct
DVVMTQTPLTLSVTLGQPASISCRSSQSLLDSDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 113 - protein, 121 aa, synthetic construct
EVQLQQSGPELVKPGASVKISCKASGYSITGYYMHWVKQSHVKSLEWIGRINPYTGATSYTQNFKDKASLTVDKSSSTAYMELLSLKSEDSAVYYCAKGGGRSSYWYFDVWGAGTAVTVSS

SEQ ID NO: 114 - protein, 107 aa, synthetic construct
DIQMTQTTSSLSASLGDRVTISCSASQAISNYLNWYQQKPDGTIKLLIYYTSSLHSGVPSRFSGSGSGTDYSLTISNLEPEDIATYYCQQYSKLPWTFGGGTKLEIK

SEQ ID NO: 115 - protein, 123 aa, synthetic construct
QIQLVQSGPELKKPGETVKISCKASGYTFTDFSMHWVKQAPGKGLKWMGWINTETVEPTYADDFKGRFAFSLETSASTAYLQINNLKNEDTATYFCSRVSYDGYYGDFAMDYWSQGTSVTVSS

SEQ ID NO: 116 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 117-124 - skipped (8 entries)

SEQ ID NO: 125 - DNA, 357 bp, synthetic construct
  source 1..357: mol_type = other DNA
cagatccagttggtgcagtctggacctgagctgaagaagcctggagagacagtcaagatctcctgcaaggcttctggttataccttcacagactattcaatgcactgggtgaagcagactccaggaaagggtttaaagtggatgggctggataaatactgagactggtgagccaacatatgcagatgacttcaagggacggtttgccttctctttggaaacctctgccagcactgcctatttgcagatcaacaacctcaaaaatgaggacacggctacatatttctgtgctagaaactactatgatacgaccgggtttgcttactggggccaagggactctggtcactgtctctgca

SEQ ID NO: 126 - DNA, 336 bp, synthetic construct
  source 1..336: mol_type = other DNA
gatgttgtgatgacccagactccactcactttgtcggttaccgttggacaaccagcctctatctcttgcaagtcaagtcagcgcctcttatatagttatggaaaaacctatttgaattggttattacagaggccaggccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaagatacacattttccgtacacgttcggaggggggaccaagctggaaataaaa

SEQ ID NO: 127 - DNA, 324 bp, synthetic construct
  source 1..324: mol_type = other DNA
gacatcttgctgactcagtctccagccatcctgtctgtgagtccaggagaaagagtcagtttctcctgcagggccagtcagagcattggcacaagcatacactggtatcagcaaagaacgaatggttctccaaggcttctcataaagtttgcttctgagtctatctctgggatcccttccaggtttagtggcagtggatcagggacagattttcctcttagcatcaacagtgtggagtctgaagatattgcaggttattactgtcaacaaagtaatagctggccaacgtacacgttcggaggggggaccaagctggaaataaaa

SEQ ID NO: 128 - DNA, 339 bp, synthetic construct
  source 1..339: mol_type = other DNA
gaggtccacctgcaacagtctggacctgagctggtgaagcctggggcttcagtgaagatatcctgcaagacttctggatacatattcactgaatacaccatgcactgggtgaagcagagccatggaaagagccttgagtggattggaggtattaatcctaacaatggtggtactaggtacaaccagaagttcaagggcaaggccacattgactgtagacaagtcctccagcacagcctacatggagctccgcagcctgacatctgaggattctgcagtctattactgtgcttggtattttccttactggggccaagggactctggtcactgtctctgca

SEQ ID NO: 129 - DNA, 336 bp, synthetic construct
  source 1..336: mol_type = other DNA
gatgttgtgatgacccagactccactcactttgtcggttacccttggacaaccagcctccatctcttgcaggtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtggacgttcggtggaggcaccaagctggaaatcaaa

SEQ ID NO: 130 - DNA, 363 bp, synthetic construct
  source 1..363: mol_type = other DNA
gaggtccagctgcaacagtctggacctgagctggtgaagcctggggcctcagtgaagatatcctgcaaggcttctggttactcaatcactggctactacatgcactgggtgaagcaaagccatgtaaagagccttgagtggattggacgtattaatccttacactggtgctactagctacacccagaatttcaaggacaaggccagcttgactgtagataagtcctccagcacagcctacatggagctcctcagcctgaaatctgaagactctgcagtctattactgtgcgaaggggggcggtaggagctcctactggtacttcgatgtctggggcgcagggaccgcggtcaccgtctcctca

SEQ ID NO: 131 - DNA, 321 bp, synthetic construct
  source 1..321: mol_type = other DNA
gatatccagatgacacagactacatcctccctgtctgcctctctgggagacagagtcaccatcagttgcagtgcaagtcaggccattagcaattatttaaactggtatcagcagaaaccagatggaactattaaactcctgatctattacacatcaagtttacactcaggagtcccatcaaggttcagtggcagtgggtctgggacagattattctctcaccatcagcaacctggaacctgaagatattgccacttactattgtcagcagtatagtaaacttccgtggacgttcggtggaggcaccaagctggaaatcaaa

SEQ ID NO: 132 - DNA, 369 bp, synthetic construct
  source 1..369: mol_type = other DNA
cagatccagttggtgcagtctggacctgagctgaagaagcctggagagacagtcaagatctcctgcaaggcttctggttataccttcacagacttttcaatgcactgggtgaagcaggctccaggaaagggtttaaagtggatgggctggataaacactgagactgttgagccaacatatgcagatgacttcaagggacggtttgccttctctttggaaacctctgccagcactgcctatttgcagatcaacaacctcaaaaatgaggacacggctacatatttctgttctagagtctcctatgatggttactatggggactttgctatggactactggagtcaaggaacctcagtcaccgtctcctca

SEQ ID NO: 133 - DNA, 321 bp, synthetic construct
  source 1..321: mol_type = other DNA
gatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccttggacgttcggtggaggcaccaagctggaaatcaaa

SEQ ID NO: 134 - protein, 5 aa, synthetic construct
SYWMS

SEQ ID NO: 135 - protein, 17 aa, synthetic construct
RIDPYDSETHYNQKFKD

SEQ ID NO: 136 - protein, 7 aa, synthetic construct
TAASFDY

SEQ ID NO: 137 - protein, 5 aa, synthetic construct
NYWMN

SEQ ID NO: 138 - protein, 19 aa, synthetic construct
EIRLKSNNYATHYAESVKG

SEQ ID NO: 139 - skipped

SEQ ID NO: 140 - protein, 5 aa, synthetic construct
SYTMH

SEQ ID NO: 141 - protein, 17 aa, synthetic construct
YINPSSGYTDYNQKFKD

SEQ ID NO: 142 - protein, 12 aa, synthetic construct
LYDNYDYYAMDY

SEQ ID NO: 143 - protein, 5 aa, synthetic construct
SYGVH

SEQ ID NO: 144 - protein, 16 aa, synthetic construct
VIWSGGTTDYNAAFIS

SEQ ID NO: 145 - protein, 8 aa, synthetic construct
GGHRGFAY

SEQ ID NO: 146 - protein, 5 aa, synthetic construct
DYSMH

SEQ ID NO: 147 - protein, 17 aa, synthetic construct
WINTETGEPTYADDFKG

SEQ ID NO: 148 - protein, 10 aa, synthetic construct
NYYDTTGFAY

SEQ ID NO: 149 - protein, 5 aa, synthetic construct
EYTMH

SEQ ID NO: 150 - protein, 17 aa, synthetic construct
GINPNNGGTRYNQKFKG

SEQ ID NO: 151 - protein, 4 aa, synthetic construct
YFPY

SEQ ID NO: 152 - protein, 5 aa, synthetic construct
GYYMH

SEQ ID NO: 153 - protein, 17 aa, synthetic construct
RINPYTGATSYTQNFKD

SEQ ID NO: 154 - protein, 12 aa, synthetic construct
GGGRSSYWYFDV

SEQ ID NO: 155 - protein, 5 aa, synthetic construct
DFSMH

SEQ ID NO: 156 - protein, 17 aa, synthetic construct
WINTETVEPTYADDFKG

SEQ ID NO: 157 - protein, 14 aa, synthetic construct
VSYDGYYGDFAMDY

SEQ ID NO: 158 - protein, 16 aa, synthetic construct
RSSQSLVHSNGNSYLH

SEQ ID NO: 159 - protein, 7 aa, synthetic construct
KVSNRFS

SEQ ID NO: 160 - protein, 9 aa, synthetic construct
SQSTHVPFT

SEQ ID NO: 161 - protein, 12 aa, synthetic construct
SASSTISSNYLH

SEQ ID NO: 162 - protein, 7 aa, synthetic construct
RTSNLAS

SEQ ID NO: 163 - protein, 9 aa, synthetic construct
QQGSTISRT

SEQ ID NO: 164 - protein, 17 aa, synthetic construct
KSSQSLLNSRIRKNYLA

SEQ ID NO: 165 - protein, 7 aa, synthetic construct
WASTRES

SEQ ID NO: 166 - protein, 8 aa, synthetic construct
KQSYNLLT

SEQ ID NO: 167 - protein, 11 aa, synthetic construct
KASQDVGTAVA

SEQ ID NO: 168 - protein, 7 aa, synthetic construct
WASTRHT

SEQ ID NO: 169 - protein, 9 aa, synthetic construct
QQYSSYPLT

SEQ ID NO: 170 - protein, 16 aa, synthetic construct
KSSQRLLYSYGKTYLN

SEQ ID NO: 171 - protein, 7 aa, synthetic construct
LVSKLDS

SEQ ID NO: 172 - protein, 9 aa, synthetic construct
VQDTHFPYT

SEQ ID NO: 173 - protein, 11 aa, synthetic construct
RASQSIGTSIH

SEQ ID NO: 174 - protein, 7 aa, synthetic construct
FASESIS

SEQ ID NO: 175 - protein, 10 aa, synthetic construct
QQSNSWPTYT

SEQ ID NO: 176 - protein, 16 aa, synthetic construct
RSSQSLLDSDGKTYLN

SEQ ID NO: 177 - protein, 7 aa, synthetic construct
LVSKLDS

SEQ ID NO: 178 - protein, 9 aa, synthetic construct
WQGTHFPWT

SEQ ID NO: 179 - protein, 11 aa, synthetic construct
SASQAISNYLN

SEQ ID NO: 180 - protein, 7 aa, synthetic construct
YTSSLHS

SEQ ID NO: 181 - protein, 9 aa, synthetic construct
QQYSKLPWT

SEQ ID NO: 182 - protein, 11 aa, synthetic construct
RASQDISNYLN

SEQ ID NO: 183 - protein, 7 aa, synthetic construct
YTSRLHS

SEQ ID NO: 184 - protein, 9 aa, synthetic construct
QQGNTLPWT

SEQ ID NO: 185 - DNA, 15 bp, synthetic construct
  source 1..15: mol_type = other DNA
agctactggatgagc

SEQ ID NO: 186 - DNA, 51 bp, synthetic construct
  source 1..51: mol_type = other DNA
aggattgatccttacgatagtgaaactcactacaatcaaaagttcaaggac

SEQ ID NO: 187 - DNA, 21 bp, synthetic construct
  source 1..21: mol_type = other DNA
acggccgcttcgtttgactat

SEQ ID NO: 188 - DNA, 15 bp, synthetic construct
  source 1..15: mol_type = other DNA
aactactggatgaac

SEQ ID NO: 189 - DNA, 57 bp, synthetic construct
  source 1..57: mol_type = other DNA
gaaattagattgaaatctaataattatgcaacacattatgcggagtctgtgaaaggg

SEQ ID NO: 190 - skipped

SEQ ID NO: 191 - DNA, 15 bp, synthetic construct
  source 1..15: mol_type = other DNA
agctacacgatgcac

SEQ ID NO: 192 - DNA, 51 bp, synthetic construct
  source 1..51: mol_type = other DNA
tacattaatcctagcagtggatatactgattacaatcagaagttcaaggac

SEQ ID NO: 193 - DNA, 36 bp, synthetic construct
  source 1..36: mol_type = other DNA
ctatatgataactacgattactatgctatggactac

SEQ ID NO: 194 - DNA, 15 bp, synthetic construct
  source 1..15: mol_type = other DNA
agctatggtgtacac

SEQ ID NO: 195 - DNA, 48 bp, synthetic construct
  source 1..48: mol_type = other DNA
gtgatatggagtggtggaactacagactataatgcagctttcatatcc

SEQ ID NO: 196 - DNA, 24 bp, synthetic construct
  source 1..24: mol_type = other DNA
ggggggcaccgagggtttgcttac

SEQ ID NO: 197 - DNA, 15 bp, synthetic construct
  source 1..15: mol_type = other DNA
gactattcaatgcac

SEQ ID NO: 198 - DNA, 51 bp, synthetic construct
  source 1..51: mol_type = other DNA
tggataaatactgagactggtgagccaacatatgcagatgacttcaaggga

SEQ ID NO: 199 - DNA, 30 bp, synthetic construct
  source 1..30: mol_type = other DNA
aactactatgatacgaccgggtttgcttac

SEQ ID NO: 200 - DNA, 15 bp, synthetic construct
  source 1..15: mol_type = other DNA
gaatacaccatgcac

SEQ ID NO: 201 - DNA, 51 bp, synthetic construct
  source 1..51: mol_type = other DNA
ggtattaatcctaacaatggtggtactaggtacaaccagaagttcaagggc

SEQ ID NO: 202 - DNA, 12 bp, synthetic construct
  source 1..12: mol_type = other DNA
tattttccttac

SEQ ID NO: 203 - DNA, 15 bp, synthetic construct
  source 1..15: mol_type = other DNA
ggctactacatgcac

SEQ ID NO: 204 - DNA, 51 bp, synthetic construct
  source 1..51: mol_type = other DNA
cgtattaatccttacactggtgctactagctacacccagaatttcaaggac

SEQ ID NO: 205 - DNA, 36 bp, synthetic construct
  source 1..36: mol_type = other DNA
gggggcggtaggagctcctactggtacttcgatgtc

SEQ ID NO: 206 - DNA, 15 bp, synthetic construct
  source 1..15: mol_type = other DNA
gacttttcaatgcac

SEQ ID NO: 207 - DNA, 51 bp, synthetic construct
  source 1..51: mol_type = other DNA
tggataaacactgagactgttgagccaacatatgcagatgacttcaaggga

SEQ ID NO: 208 - DNA, 42 bp, synthetic construct
  source 1..42: mol_type = other DNA
gtctcctatgatggttactatggggactttgctatggactac

SEQ ID NO: 209 - DNA, 48 bp, synthetic construct
  source 1..48: mol_type = other DNA
agatctagtcagagccttgtacacagtaatggaaactcctatttacat

SEQ ID NO: 210 - DNA, 21 bp, synthetic construct
  source 1..21: mol_type = other DNA
aaagtttccaaccgattttct

SEQ ID NO: 211 - DNA, 27 bp, synthetic construct
  source 1..27: mol_type = other DNA
tctcaaagtacacatgttccattcacg

SEQ ID NO: 212 - DNA, 36 bp, synthetic construct
  source 1..36: mol_type = other DNA
agtgccagctcaactataagttccaattacttgcat

SEQ ID NO: 213 - DNA, 21 bp, synthetic construct
  source 1..21: mol_type = other DNA
aggacatccaatctggcttct

SEQ ID NO: 214 - DNA, 27 bp, synthetic construct
  source 1..27: mol_type = other DNA
cagcagggtagtactatatcacgcacg

SEQ ID NO: 215 - DNA, 51 bp, synthetic construct
  source 1..51: mol_type = other DNA
aaatccagtcagagtctgctcaatagtagaatccgaaagaactacttggct

SEQ ID NO: 216 - DNA, 21 bp, synthetic construct
  source 1..21: mol_type = other DNA
tgggcatccactagggaatct

SEQ ID NO: 217 - DNA, 24 bp, synthetic construct
  source 1..24: mol_type = other DNA
aagcaatcttataatctgctcacg

SEQ ID NO: 218 - DNA, 33 bp, synthetic construct
  source 1..33: mol_type = other DNA
aaggccagtcaagatgtgggtactgctgtagcc

SEQ ID NO: 219 - DNA, 21 bp, synthetic construct
  source 1..21: mol_type = other DNA
tgggcatccacccggcacact

SEQ ID NO: 220 - DNA, 27 bp, synthetic construct
  source 1..27: mol_type = other DNA
cagcaatatagcagctatcctctcacg

SEQ ID NO: 221 - DNA, 48 bp, synthetic construct
  source 1..48: mol_type = other DNA
aagtcaagtcagcgcctcttatatagttatggaaaaacctatttgaat

SEQ ID NO: 222 - DNA, 21 bp, synthetic construct
  source 1..21: mol_type = other DNA
ctggtgtctaaactggactct

SEQ ID NO: 223 - DNA, 27 bp, synthetic construct
  source 1..27: mol_type = other DNA
gtgcaagatacacattttccgtacacg

SEQ ID NO: 224 - DNA, 33 bp, synthetic construct
  source 1..33: mol_type = other DNA
agggccagtcagagcattggcacaagcatacac

SEQ ID NO: 225 - DNA, 21 bp, synthetic construct
  source 1..21: mol_type = other DNA
tttgcttctgagtctatctct

SEQ ID NO: 226 - DNA, 30 bp, synthetic construct
  source 1..30: mol_type = other DNA
caacaaagtaatagctggccaacgtacacg

SEQ ID NO: 227 - DNA, 48 bp, synthetic construct
  source 1..48: mol_type = other DNA
aggtcaagtcagagcctcttagatagtgatggaaagacatatttgaat

SEQ ID NO: 228 - DNA, 21 bp, synthetic construct
  source 1..21: mol_type = other DNA
ctggtgtctaaactggactct

SEQ ID NO: 229 - DNA, 27 bp, synthetic construct
  source 1..27: mol_type = other DNA
tggcaaggtacacattttccgtggacg

SEQ ID NO: 230 - DNA, 33 bp, synthetic construct
  source 1..33: mol_type = other DNA
agtgcaagtcaggccattagcaattatttaaac

SEQ ID NO: 231 - DNA, 21 bp, synthetic construct
  source 1..21: mol_type = other DNA
tacacatcaagtttacactca

SEQ ID NO: 232 - DNA, 27 bp, synthetic construct
  source 1..27: mol_type = other DNA
cagcagtatagtaaacttccgtggacg

SEQ ID NO: 233 - DNA, 33 bp, synthetic construct
  source 1..33: mol_type = other DNA
agggcaagtcaggacattagcaattatttaaac

SEQ ID NO: 234 - DNA, 21 bp, synthetic construct
  source 1..21: mol_type = other DNA
tacacatcaagattacactca

SEQ ID NO: 235 - DNA, 27 bp, synthetic construct
  source 1..27: mol_type = other DNA
caacagggtaatacgcttccttggacg

SEQ ID NO: 236 - protein, 15 aa, synthetic construct
  VARIANT 1: note = Residue may be deleted
CVPLDGKTITNNQRI

SEQ ID NO: 237 - protein, 15 aa, synthetic construct
  VARIANT 1: note = Residue may be deleted
CVPLDGKTITNNQRI

SEQ ID NO: 238 - DNA, 1407 bp, synthetic construct
  source 1..1407: mol_type = other DNA
atggcttgggtgtggaccttgctattcctgatggcagctgcccaaagtatccaagcacagatccagttggtgcagtctggacctgagctgaagaagcctggagagacagtcaagatctcctgcaaggcttctggttataccttcacagactattcaatgcactgggtgaagcagactccaggaaagggtttaaagtggatgggctggataaatactgagactggtgagccaacatatgcagatgacttcaagggacggtttgccttctctttggaaacctctgccagcactgcctatttgcagatcaacaacctcaaaaatgaggacacggctacatatttctgtgctagaaactactatgatacgaccgggtttgcttactggggccaagggactctggtcactgtctctgcagctacaacaacagccccatctgtctatcccttggtccctggctgcagtgacacatctggatcctcggtgacactgggatgccttgtcaaaggctacttccctgagccggtaactgtaaaatggaactatggagccctgtccagcggtgtgcgcacagtctcatctgtcctgcagtctgggttctattccctcagcagcttggtgactgtaccctccagcacctggcccagccagactgtcatctgcaacgtagcccacccagccagcaagactgagttgatcaagagaatcgagcctagaatacccaagcccagtacccccccaggttcttcatgcccacctggtaacatcttgggtggaccatccgtcttcatcttccccccaaagcccaaggatgcactcatgatctccctaacccccaaggttacgtgtgtggtggtggatgtgagcgaggatgacccagatgtccatgtcagctggtttgtggacaacaaagaagtacacacagcctggacacagccccgtgaagctcagtacaacagtaccttccgagtggtcagtgccctccccatccagcaccaggactggatgaggggcaaggagttcaaatgcaaggtcaacaacaaagccctcccagcccccatcgagagaaccatctcaaaacccaaaggaagagcccagacacctcaagtatacaccatacccccacctcgtgaacaaatgtccaagaagaaggttagtctgacctgcctggtcaccaacttcttctctgaagccatcagtgtggagtgggaaaggaacggagaactggagcaggattacaagaacactccacccatcctggactcagatgggacctacttcctctacagcaagctcactgtggatacagacagttggttgcaaggagaaatttttacctgctccgtggtgcatgaggctctccataaccaccacacacagaagaacctgtctcgctcccctggtaaatga

SEQ ID NO: 239 - protein, 468 aa, synthetic construct
MAWVWTLLFLMAAAQSIQAQIQLVQSGPELKKPGETVKISCKASGYTFTDYSMHWVKQTPGKGLKWMGWINTETGEPTYADDFKGRFAFSLETSASTAYLQINNLKNEDTATYFCARNYYDTTGFAYWGQGTLVTVSAATTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 240 - DNA, 717 bp, synthetic construct
  source 1..717: mol_type = other DNA
atgagtcctgcccagttcctgtttctgttagtgctctggattcaggaaaccaaaggtgatgttgtgatgacccagactccactcactttgtcggttaccgttggacaaccagcctctatctcttgcaagtcaagtcagcgcctcttatatagttatggaaaaacctatttgaattggttattacagaggccaggccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaagatacacattttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 241 - protein, 238 aa, synthetic construct
MSPAQFLFLLVLWIQETKGDVVMTQTPLTLSVTVGQPASISCKSSQRLLYSYGKTYLNWLLQRPGQSPKRLIYLVSKLDSGVPDRFTGSGSGTDFTLKISRVEAEDLGVYYCVQDTHFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 242 - DNA, 708 bp, synthetic construct
  source 1..708: mol_type = other DNA
atggtatccacacctcagttccttgtatttttgcttttctggattccagcctccagaggtgacatcttgctgactcagtctccagccatcctgtctgtgagtccaggagaaagagtcagtttctcctgcagggccagtcagagcattggcacaagcatacactggtatcagcaaagaacgaatggttctccaaggcttctcataaagtttgcttctgagtctatctctgggatcccttccaggtttagtggcagtggatcagggacagattttcctcttagcatcaacagtgtggagtctgaagatattgcaggttattactgtcaacaaagtaatagctggccaacgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 243 - protein, 235 aa, synthetic construct
MVSTPQFLVFLLFWIPASRGDILLTQSPAILSVSPGERVSFSCRASQSIGTSIHWYQQRTNGSPRLLIKFASESISGIPSRFSGSGSGTDFPLSINSVESEDIAGYYCQQSNSWPTYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 244 - DNA, 1389 bp, synthetic construct
  source 1..1389: mol_type = other DNA
atgggatggagctggatctttctctttctcctgtcaggaactgcaggtgtcctctctgaggtccacctgcaacagtctggacctgagctggtgaagcctggggcttcagtgaagatatcctgcaagacttctggatacatattcactgaatacaccatgcactgggtgaagcagagccatggaaagagccttgagtggattggaggtattaatcctaacaatggtggtactaggtacaaccagaagttcaagggcaaggccacattgactgtagacaagtcctccagcacagcctacatggagctccgcagcctgacatctgaggattctgcagtctattactgtgcttggtattttccttactggggccaagggactctggtcactgtctctgcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 245 - protein, 462 aa, synthetic construct
MGWSWIFLFLLSGTAGVLSEVHLQQSGPELVKPGASVKISCKTSGYIFTEYTMHWVKQSHGKSLEWIGGINPNNGGTRYNQKFKGKATLTVDKSSSTAYMELRSLTSEDSAVYYCAWYFPYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 246 - DNA, 717 bp, synthetic construct
  source 1..717: mol_type = other DNA
atgagtcctgcccagttcctgtttctgttagtgctctggattcgggaaaccaacggtgatgttgtgatgacccagactccactcactttgtcggttacccttggacaaccagcctccatctcttgcaggtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 247 - protein, 238 aa, synthetic construct
MSPAQFLFLLVLWIRETNGDVVMTQTPLTLSVTLGQPASISCRSSQSLLDSDGKTYLNWLLQRPGQSPKRLIYLVSKLDSGVPDRFTGSGSGTDFTLKISRVEAEDLGVYYCWQGTHFPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 248 - DNA, 1431 bp, synthetic construct
  source 1..1431: mol_type = other DNA
atgggatggagctggatctttctctttctcctgtcaggaactgcaggtgtcctctctgaggtccagctgcaacagtctggacctgagctggtgaagcctggggcctcagtgaagatatcctgcaaggcttctggttactcaatcactggctactacatgcactgggtgaagcaaagccatgtaaagagccttgagtggattggacgtattaatccttacactggtgctactagctacacccagaatttcaaggacaaggccagcttgactgtagataagtcctccagcacagcctacatggagctcctcagcctgaaatctgaagactctgcagtctattactgtgcgaaggggggcggtaggagctcctactggtacttcgatgtctggggcgcagggaccgcggtcaccgtctcctcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaatga

SEQ ID NO: 249 - protein, 476 aa, synthetic construct
MGWSWIFLFLLSGTAGVLSEVQLQQSGPELVKPGASVKISCKASGYSITGYYMHWVKQSHVKSLEWIGRINPYTGATSYTQNFKDKASLTVDKSSSTAYMELLSLKSEDSAVYYCAKGGGRSSYWYFDVWGAGTAVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 250 - DNA, 702 bp, synthetic construct
  source 1..702: mol_type = other DNA
atgtcctctgctcagttccttggtctcctattgctctgttttcaaggtaccagatgtgatatccagatgacacagactacatcctccctgtctgcctctctgggagacagagtcaccatcagttgcagtgcaagtcaggccattagcaattatttaaactggtatcagcagaaaccagatggaactattaaactcctgatctattacacatcaagtttacactcaggagtcccatcaaggttcagtggcagtgggtctgggacagattattctctcaccatcagcaacctggaacctgaagatattgccacttactattgtcagcagtatagtaaacttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 251 - protein, 233 aa, synthetic construct
MSSAQFLGLLLLCFQGTRCDIQMTQTTSSLSASLGDRVTISCSASQAISNYLNWYQQKPDGTIKLLIYYTSSLHSGVPSRFSGSGSGTDYSLTISNLEPEDIATYYCQQYSKLPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 252 - DNA, 1419 bp, synthetic construct
  source 1..1419: mol_type = other DNA
atggcttgggtgtggaccttgctattcctgatggcagctgcccaaagtatccaagcacagatccagttggtgcagtctggacctgagctgaagaagcctggagagacagtcaagatctcctgcaaggcttctggttataccttcacagacttttcaatgcactgggtgaagcaggctccaggaaagggtttaaagtggatgggctggataaacactgagactgttgagccaacatatgcagatgacttcaagggacggtttgccttctctttggaaacctctgccagcactgcctatttgcagatcaacaacctcaaaaatgaggacacggctacatatttctgttctagagtctcctatgatggttactatggggactttgctatggactactggagtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 253 - protein, 472 aa, synthetic construct
MAWVWTLLFLMAAAQSIQAQIQLVQSGPELKKPGETVKISCKASGYTFTDFSMHWVKQAPGKGLKWMGWINTETVEPTYADDFKGRFAFSLETSASTAYLQINNLKNEDTATYFCSRVSYDGYYGDFAMDYWSQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 254 - DNA, 705 bp, synthetic construct
  source 1..705: mol_type = other DNA
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccttggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 255 - protein, 234 aa, synthetic construct
MMSSAQFLGLLLLCFQGTRCDIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 256 - DNA, 348 bp, synthetic construct
  source 1..348: mol_type = other DNA
caggtccaactgcagcagcctggggctgaggtggtgaggcctggggcttcagtgaaggtgtcctgcaaggcttctggctacacggtcagcagctactggatgagctgggttaagcagaggccggagcaaggccttgagtggattggaaggattgatccttacgatagtgaaactcactacaatcaaaagttcaaggacaaggccatattgactgtagacaaatcctccagcacagcctacatgcaactcagcagcctgacatctgaggactctgcggtctattactgtgcaaggacggccgcttcgtttgactattggggccaaggcaccactctcacagtctcctca

SEQ ID NO: 257 - protein, 116 aa, synthetic construct
QVQLQQPGAEVVRPGASVKVSCKASGYTVSSYWMSWVKQRPEQGLEWIGRIDPYDSETHYNQKFKDKAILTVDKSSSTAYMQLSSLTSEDSAVYYCARTAASFDYWGQGTTLTVSS

SEQ ID NO: 258 - DNA, 336 bp, synthetic construct
  source 1..336: mol_type = other DNA
gatgttgtgatgacccaaactccactctccctgcctgtcagtcttggagatcaagcctccatctcttgcagatctagtcagagccttgtacacagtaatggaaactcctatttacattggtacctgcagaagccaggccagtctccaaagctcctgatctacaaagtttccaaccgattttctggggtcccagacaggttcagtggcagtggatcagggacagatttcacactcaatatcagcagagtggaggctgaggatctgggagtttatttctgctctcaaagtacacatgttccattcacgttcggctcggggacaaagttggaaataaaa

SEQ ID NO: 259 - protein, 112 aa, synthetic construct
DVVMTQTPLSLPVSLGDQASISCRSSQSLVHSNGNSYLHWYLQKPGQSPKLLIYKVSNRFSGVPDRFSGSGSGTDFTLNISRVEAEDLGVYFCSQSTHVPFTFGSGTKLEIK

SEQ ID NO: 260 - DNA, 342 bp, synthetic construct
  source 1..342: mol_type = other DNA
gaagtgaagcttgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagcgatgattccaaaagtagtgtctacctgcaaatgaacaacttaagagctgaagacactggcatttattactgttcaactgggaactactggggccaaggcaccactctcacagtctcctca

SEQ ID NO: 261 - protein, 114 aa, synthetic construct
EVKLEESGGGLVQPGGSMKLSCVASGFTFSNYWMNWVRQSPEKGLEWVAEIRLKSNNYATHYAESVKGRFTISSDDSKSSVYLQMNNLRAEDTGIYYCSTGNYWGQGTTLTVSS

SEQ ID NO: 262 - DNA, 324 bp, synthetic construct
  source 1..324: mol_type = other DNA
gaaattgtgctcacccagtctccaaccaccatggctgcatctcccggggagaagatcactatcacctgcagtgccagctcaactataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtactatatcacgcacgttcggctcggggacaaagttggaaataaaa

SEQ ID NO: 263 - protein, 108 aa, synthetic construct
EIVLTQSPTTMAASPGEKITITCSASSTISSNYLHWYQQKPGFSPKLLIYRTSNLASGVPARFSGSGSGTSYSLTIGTMEAEDVATYYCQQGSTISRTFGSGTKLEIK

SEQ ID NO: 264 - DNA, 363 bp, synthetic construct
  source 1..363: mol_type = other DNA
caggtccagctgcagcagtctgcagctgaactggcaagacctggggcctcagtgaagatgtcctgcaaggcttctggctacacctttagtagctacacgatgcactgggtaaaacagaggcctggacagggtctggaatggattggatacattaatcctagcagtggatatactgattacaatcagaagttcaaggacaagaccacattgactgcagacaaatcctccagcacagcctacatgcaactgagcagcctgacatctgaggactctgcggtctattactgtgcaagactatatgataactacgattactatgctatggactactggggtcaaggaacctcagtcaccgtctcctca

SEQ ID NO: 265 - protein, 121 aa, synthetic construct
QVQLQQSAAELARPGASVKMSCKASGYTFSSYTMHWVKQRPGQGLEWIGYINPSSGYTDYNQKFKDKTTLTADKSSSTAYMQLSSLTSEDSAVYYCARLYDNYDYYAMDYWGQGTSVTVSS

SEQ ID NO: 266 - DNA, 336 bp, synthetic construct
  source 1..336: mol_type = other DNA
gacattgtgatgtcacagtctccatcctccctggctgtgtcagcaggagagaaggtcactatgagctgcaaatccagtcagagtctgctcaatagtagaatccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgatgacctggcagtttattactgcaagcaatcttataatctgctcacgttcggtgctgggaccaagctggagctgaaa

SEQ ID NO: 267 - protein, 112 aa, synthetic construct
DIVMSQSPSSLAVSAGEKVTMSCKSSQSLLNSRIRKNYLAWYQQKPGQSPKLLIYWASTRESGVPDRFTGSGSGTDFTLTISSVQADDLAVYYCKQSYNLLTFGAGTKLELK

SEQ ID NO: 268 - DNA, 348 bp, synthetic construct
  source 1..348: mol_type = other DNA
caggtgcagctgaagcagtcaggacctggcctagtgcagccctcacagagcctgtccatcacctgcacagtctctggtttctcattaactagctatggtgtacactgggttcgccagtctccaggaaagggtctggagtggctgggagtgatatggagtggtggaactacagactataatgcagctttcatatccagactgagcatcagcaaggacaattccaagagccaagttttctttaaaatgaacagtctgcaagctaatgacacagccatatattactgtgccagaggggggcaccgagggtttgcttactggggccaagggactctggtcactgtctctgca

SEQ ID NO: 269 - protein, 116 aa, synthetic construct
QVQLKQSGPGLVQPSQSLSITCTVSGFSLTSYGVHWVRQSPGKGLEWLGVIWSGGTTDYNAAFISRLSISKDNSKSQVFFKMNSLQANDTAIYYCARGGHRGFAYWGQGTLVTVSA

SEQ ID NO: 270 - DNA, 321 bp, synthetic construct
  source 1..321: mol_type = other DNA
gacattgtgatgacccagtctcacaaattcatgtccacatcagtaggagacagggtcagcatcacctgcaaggccagtcaagatgtgggtactgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcctctcacgttcggtgctgggaccaagctggagctgaaa

SEQ ID NO: 271 - protein, 107 aa, synthetic construct
DIVMTQSHKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLELK

SEQ ID NO: 272 - protein, 28 aa, synthetic construct
  VARIANT 1: note = Residue may be deleted
  VARIANT 16: note = Residue may be deleted
CFWVNPDCGLKTRKKCAILGGSKVSDKI

SEQ ID NO: 273 - protein, 28 aa, synthetic construct
  VARIANT 1: note = Residue may be deleted
  VARIANT 16: note = Residue may be deleted
CAILGGSKVSDKIKKCFWVNPDCGLKTR

SEQ ID NO: 274 - protein, 35 aa, synthetic construct
  VARIANT 20: note = Residue may be deleted
RGDIAEALDIFHTKGQLKKCFWVNPDCGLKTRKLH